(19) United States
(12) Patent Application Publication
(10) Pub. No.: US 2024/0350645 A1
(43) Pub. Date: Oct. 24, 2024

(54) AUTOMATED SYNTHESIS OF POLYMERIC DRUGS

(71) Applicant: SONY GROUP CORPORATION, Tokyo (JP)

(72) Inventors: Tracy MATRAY, Snohomish, WA (US); Michael VANBRUNT, Bothell, WA (US); John Michael MCCUTCHEON, Shoreline, WA (US)

(21) Appl. No.: 18/290,630

(22) PCT Filed: Jul. 21, 2022
(86) PCT No.: PCT/IB2022/056757

§ 371 Date: Jan. 19, 2024

Related U.S. Application Data
(60) Provisional application No. 63/224,777, filed on Jul. 22, 2021.
(60) Provisional application No. 63/250,892, filed on Sep. 30, 2021.
(60) Provisional application No. 63/252,993, filed on Oct. 6, 2021.

(51) Int. Cl. A61K 47/58; A61K 31/4745; A61K 47/68; A61P 35/00; C07C 237/12; C07D 207/09; C07D 491/22; C07F 9/24; C07F 9/6558; C07F 9/6561

(52) U.S. Cl. CPC A61K 47/605; A61K 31/4745; A61K 47/6849; A61K 47/6855; A61P 35/00; C07C 237/12; C07D 207/09; C07D 491/22; C07F 9/24; C07F 9/65586; C07F 9/6561

(57) ABSTRACT

Compounds useful as biologically active compounds are disclosed. The compounds have the following structure (I): or a stereoisomer, tautomer or salt thereof, wherein L¹, L², L³, R¹ R², M, p, q, m, and n are as defined herein. Additional compounds, methods of preparation, pharmaceutical compositions, and methods of treatment related to compounds of Structure (I) are also provided.

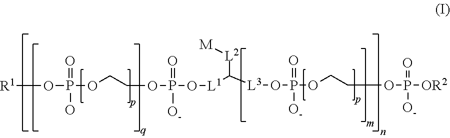

### BACKGROUND

### Technical Field

Embodiments of the present disclosure are directed to methods for preparing dimeric and polymeric biologically active compounds having spacing groups, compounds and methods of treatment related to the same.

### Description of the Related Art

Targeted drug conjugates, unlike, e.g., chemotherapy, deliver drugs to target cells, with little or no off-target activity. Typically, targeted drug conjugates comprise a targeting molecule that is linked to a biologically active payload or drug. By combining the unique targeting capability with the therapeutic effectiveness of a biologically active drug, conjugates can deliver the drug only to the intended target and minimize potential side effects.

Antibody-drug conjugates (ADCs) are one class of targeted drug conjugates that are of particular interest, for example for cancer treatment. ADCs for cancer treatment combine the targeting features of monoclonal antibodies with cancer-killing ability of cytotoxic agents to provide a therapeutic with several advantages over other chemotherapeutics. However, challenges related to the complexity of ADC constructs, specifically the chemical linker between antibody and drug, has caused significant difficulties for development of new and effective therapeutics. Although the first ADC was approved in 2001, it took almost a decade before the next ADC was approved. As of today, only Adcetris®, Besponsa®, Enhertu®, Mylotarg®, Padcev®, Polivy®, and Kadcyla® are commercially available globally (Zevalin® has been approved in China only).

Thus, there exists a need in the art for developing potent, targeting drug conjugates having a high therapeutic index and methods of preparing the same. The present disclosure fulfills this need and provides further related advantages.

### BRIEF SUMMARY

One embodiment provides a compound having the following Structure (I):

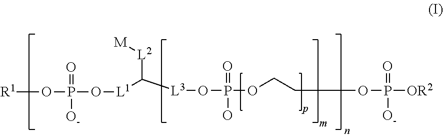

or a pharmaceutically acceptable salt, stereoisomer, or tautomer thereof, wherein L¹, L², L³, R¹, R², M, p, m, and n are as defined herein.

Another embodiment provides a compound having the following Structure (I):

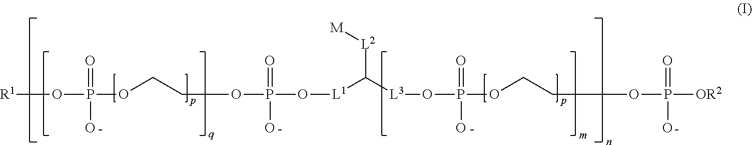

or a pharmaceutically acceptable salt, stereoisomer, or tautomer thereof, wherein L¹, L², L³, R¹, R², M, p, q, m, and n are as defined herein.

Another embodiment provides a method for preparing a compound of Structure (I) or a pharmaceutically acceptable salt, stereoisomer, or tautomer thereof, wherein L¹, L², L³, R¹, R², M, p, m, and n are as defined herein.

One more embodiment provides a compound having the following Structure (II):

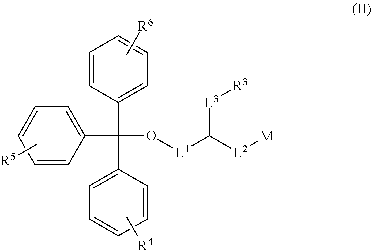

or salt, tautomer, or stereoisomer thereof, wherein L¹, L², L³, R³, R⁴, R⁵, R⁶, and M are as defined herein.

Still other embodiments provide a method of treating a disease or disorder (e.g., cancer), the method comprising administering to a subject in need thereof a therapeutically effective amount of a compound of Structure (I). These and other aspects of the disclosure will be apparent upon reference to the following detailed description.

### DETAILED DESCRIPTION

In the following description, certain specific details are set forth in order to provide a thorough understanding of various embodiments of the disclosure. However, one skilled in the art will understand that embodiments of the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Alkylene” or “alkylene chain” refers to a straight or branched divalent hydrocarbon chain linking the rest of the molecule to a radical group, consisting solely of carbon and hydrogen, containing no unsaturation, and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, alkylene is optionally substituted.

“Heteroalkylene” refers to an alkylene group, as defined above, comprising at least one heteroatom (e.g., Si, N, O, P or S) within the alkylene chain or at a terminus of the alkylene chain. In some embodiments, the heteroatom is within the alkylene chain (i.e., the heteroalkylene comprises at least one carbon-[heteroatom]ₓ-carbon bond, where x is 1, 2 or 3). In other embodiments, the heteroatom is at a terminus of the alkylene and thus serves to join the alkylene to the remainder of the molecule (e.g., M1-H-A-M2, where M1 and M2 are portions of the molecule, H is a heteroatom and A is an alkylene). Unless stated otherwise specifically in the specification, a heteroalkylene group is optionally substituted. Exemplary heteroalkylene groups include ethylene oxide (e.g., polyethylene oxide) and the “C” linking group illustrated below:

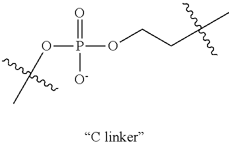 [SMILES: *CCOP(=O)([O-])O*]

A “linker” refers to a contiguous chain of at least one atom, such as carbon, oxygen, nitrogen, sulfur, phosphorous, and combinations thereof, which connects a portion of a molecule to another portion of the same molecule or to a different molecule, moiety or solid support (e.g., microparticle). Linkers may connect the molecule via a covalent bond or other means, such as ionic or hydrogen bond interactions. In some embodiments, the linker is a heteroatomic linker (e.g., comprising 1-10 Si, N, O, P, or S atoms), a heteroalkylene (e.g., comprising 1-10 Si, N, O, P, or S atoms and an alkylene chain) or an alkylene linker (e.g., comprising 1-12 carbon atoms). In some embodiments, a heteroalkylene linker comprises the following structure:

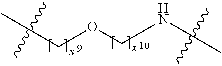

wherein:
x⁹ and x¹⁰ are each independently an integer greater than 0. In some embodiments, the heteroatomic linker is —O—, —S—, or —OP(═O)O⁻—O—. In some embodiments, the heteroalkylene linker comprises —OP(═O)O⁻—O—. In some embodiments, the heteroalkylene linker comprises at least one S—S bond.

“Physiologically cleavable linker” refers to a molecular linkage that can be split or separated a prescribed manner, resulting in two or more separate molecules while in the presence of an in vivo or in vitro environment of an organism or cell system. Generally, physiological conditions that induce such a cleavage or scission event may include a temperature ranging from about 20 to 40° C., an atmospheric pressure of about 1 atm (101 kPa or 14.7 psi), a pH of about 6 to 8, a glucose concentration of about 1 to 20 mM, atmospheric oxygen concentration, and earth gravity. In some embodiments, physiological conditions include enzymatic conditions (i.e., enzymatic cleavage). Bond cleavage or scission can be homolytic or heterolytic.

“Solid support” or “solid resin” refers to any solid substrate known in the art for solid-phase support of molecules, for example a “microparticle” refers to any of a number of small particles useful for attachment to compounds of the disclosure, including, but not limited to, glass beads, magnetic beads, polymeric beads, nonpolymeric beads, and the like. In certain embodiments, a microparticle comprises polystyrene beads. In some embodiments, the solid support or solid resin is controlled pore glass or macroporous polystyrene.

A “solid support residue” refers to the functional group remaining attached to a molecule when the molecule is cleaved from the solid support. Solid support residues are known in the art and can be easily derived based on the structure of the solid support and the group linking the molecule thereto.

Embodiments disclosed herein are also meant to encompass all compounds of Structures (I) or (II) being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively.

Isotopically-labeled compounds of Structure (I) or (II) can generally be prepared by conventional techniques known to those skilled in the art or by processes analogous to those described below and in the following Examples using an appropriate isotopically-labeled reagent in place of the non-labeled reagent previously employed.

“Stable compound” and “stable structure” are meant to indicate a compound that is sufficiently robust to survive isolation to a useful degree of purity from a reaction mixture, and formulation into an efficacious therapeutic agent.

“Salt” includes both acid and base addition salts.

“Acid addition salt” refers to those salts which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Base addition salt” refers to those salts which are prepared from addition of an inorganic base or an organic base to the free acid. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins, and the like. Particularly preferred organic bases are isopropylamine, diethylamine, ethanolamine, trimethylamine, dicyclohexylamine, choline, caffeine, and the like.

Crystallizations may produce a solvate of the compounds described herein (e.g., a compound of Structure (I) or (II)). Embodiments of the present disclosure include all solvates of the described compounds. As used herein, the term “solvate” refers to an aggregate that comprises one or more molecules of a compound of the disclosure with one or more molecules of solvent. The solvent may be water, in which case the solvate may be a hydrate. Alternatively, the solvent may be an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. The compounds of the disclosure may be true solvates, while in other cases the compounds of the disclosure may merely retain adventitious water or another solvent or be a mixture of water plus some adventitious solvent.

Embodiments of the compounds of the disclosure (e.g., compounds of Structure (I) or (II)), or their salts, tautomers or solvates may contain one or more stereocenters and may thus give rise to enantiomers, diastereomers, and other stereoisomeric forms that may be defined, in terms of absolute stereochemistry, as (R)- or (S)- or, as (D)- or (L)- for amino acids. Embodiments of the present disclosure are meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other features giving rise to geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds. Various tautomeric forms of the compounds are easily derivable by those of ordinary skill in the art.

The chemical naming protocol and structure diagrams used herein are a modified form of the I.U.P.A.C. nomenclature system, using the ACD/Name Version 9.07 software program and/or ChemDraw Ultra Version 11.0 software naming program (CambridgeSoft). Common names familiar to one of ordinary skill in the art are also used.

For ease of illustration, various compounds of Structure (I) or (II) comprising phosphorous moieties (e.g., phosphate and the like) are depicted in the anionic state (e.g., —OPO(OH)O⁻, —OPO₃²⁻). One of skill in the art will readily understand that the charge is dependent on pH and the uncharged (e.g., protonated or salt, such as sodium or other cation) forms are also included in the scope of embodiments of the disclosure.

### Compounds

As noted above, in one embodiment of the present disclosure, compounds useful as covalent linkers between biologically active moieties and targeting moieties are provided. In other embodiments, compounds useful as synthetic intermediates for preparation of compounds comprising one or more biologically active moieties are provided.

Numerous advantages are afforded by embodiments disclosed herein, including the ability to control the number of biologically active moieties that are attached to the polymer and any subsequent targeting moiety. The composition of the polymer backbone can also be selected to afford desirable solubility properties, for example, by controlling the incorporation of charged moieties (e.g., number, frequency, spacing, etc.). In addition to the properties provided by the composition of the backbone, the side chains can be selected to provide a source for tuning the solubility of the compounds disclosed herein. Monomeric units of the polymer can be selected to incorporate different anticancer therapeutics during polymer synthesis and as a post synthetic modification following polymer synthesis (e.g., coupling to an amine pendant to the polymer backbone with a therapeutic agent having an activated ester moiety).

That is, the embodiments disclosed herein also provide compounds that can advantageously include multiple therapeutic agents, for example, for complimentary or synergistic therapeutic strategies. In addition, embodiments of the present disclosure provide combinations of therapeutic agents, targeting moieties, and dye moieties (e.g., chromophores or fluorophores) that can be used for simultaneous targeting, treatment, and detection. The ease of coupling polymer-drug constructs to targeting agents such as antibodies, antibody fragments, proteins or other clinically interesting agents provides utility to a wide variety of interesting applications (e.g., surface chemistries, assay development, etc.). Accordingly, in some embodiments, M is a chromophore or fluorophore (e.g., FITC, 5-FAM, 6-FAM, and the like).

The compounds of certain embodiments also provide other desirable properties, including enhanced permeability and retention effects. In addition to providing necessary solubility characteristics, the chemical features of embodiments of the present compounds can be adjusted to modulate the compound's ability to permeate diseased cells/tissue and be retained within the same. These features allow effective delivery of biologically active agents by increasing permeation and increasing efficacy by enhancing retention.

Accordingly, it is understood that any embodiment of the compounds of Structures (I) or (II), as set forth above, may be independently combined with other embodiments to form embodiments of the disclosure not specifically set forth above. It is understood that in the present description, combinations of substituents and/or variables of the depicted formulae are permissible only if such contributions result in stable compounds.

Accordingly, one embodiment provides a compound having the following Structure (I):

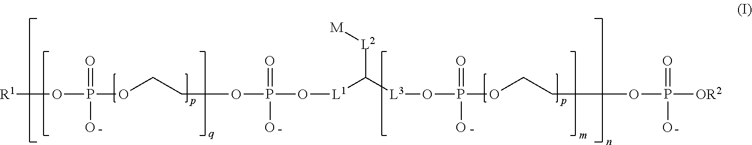

or a pharmaceutically acceptable salt, stereoisomer, or tautomer thereof, wherein
R¹ and R² are independently hydrogen, alkyl, heteroalkyl, Q or a protected form thereof, L′, or have the following structure:

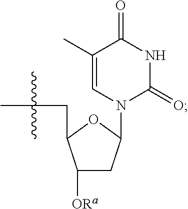

R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;

L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0;
p is an integer greater than 0; and
q is an integer greater than 0.

In another embodiment provides a compound having the following Structure (I):

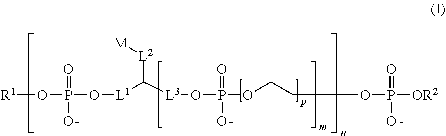

or a pharmaceutically acceptable salt, stereoisomer, or tautomer thereof, wherein
R¹ and R² are independently hydrogen, alkyl, heteroalkyl, Q or a protected form thereof, L′, or have the following structure:

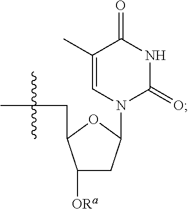

R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0; and
p is an integer greater than 0.

In some embodiments, R¹ is L′. In certain embodiments, L′ is a linker to a targeting moiety. In some more specific embodiments, L′ is a linker to a targeting moiety, the linker comprising an alkylene oxide or phosphodiester moiety, or combinations thereof. In certain specific embodiments, L′ has one of the following structures:

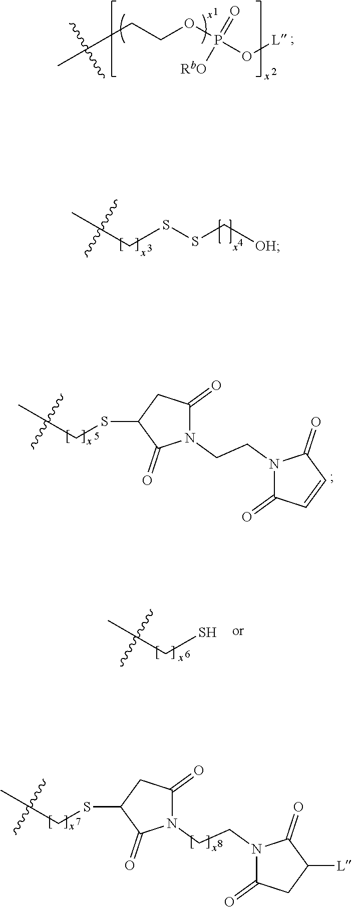

wherein:
x¹, x², x³, x⁴, x⁵, x⁶, x⁷ and x⁸ are independently an integer from 1 to 10;
R^{b} is H, an electron pair or a counter ion;
L″ is the targeting moiety or a linkage to the targeting moiety.

In some embodiments, R¹ is Q. In some of those embodiments, Q is a moiety comprising a reactive group capable of forming a covalent bond with a targeting moiety or a solid support. In other embodiments, Q is, at each occurrence, independently a moiety comprising a reactive group capable of forming a covalent bond with a complementary reactive group Q′. For example, in some embodiments, Q′ is present on a further compound of Structure (I) (e.g., in the R² or R³ position), and Q and Q′ comprise complementary reactive groups such that reaction of the compound of Structure (I) and the further compound of Structure (I) results in covalently bound dimer of the compound of Structure (I). Multimer compounds of structure (I) can also be prepared in an analogous manner and are included within the scope of embodiments of this disclosure.

The type of Q group and connectivity of the Q group to the remainder of the compound of Structure (I) is not limited, provided that Q comprises a moiety having appropriate reactivity for forming the desired bond.

In certain embodiments, Q is a moiety which is not susceptible to hydrolysis under aqueous conditions, but is sufficiently reactive to form a bond with a corresponding group on a targeting moiety or solid support (e.g., an amine, azide or alkyne).

Certain embodiments of compounds of Structure (I) comprise Q groups commonly employed in the field of bio-conjugation. For example in some embodiments, Q comprises a nucleophilic reactive group, an electrophilic reactive group or a cycloaddition reactive group. In some more specific embodiments, Q comprises a sulfhydryl, disulfide, activated ester, isothiocyanate, azide, alkyne, alkene, diene, dienophile, acid halide, sulfonyl halide, phosphine, α-haloamide, biotin, amino or maleimide functional group. In some embodiments, the activated ester is an N-succinimide ester, imidoester or polyflourophenyl ester. In other embodiments, the alkyne is an alkyl azide or acyl azide.

The Q groups can be conveniently provided in protected form to increase storage stability or other desired properties, and then the protecting group removed at the appropriate time for coupling with, for example, a targeting moiety. Accordingly, Q groups include “protected forms” of a reactive group, including any of the reactive groups described above and in the Table A below. A “protected form” of Q refers to a moiety having lower reactivity under predetermined reaction conditions relative to Q, but which can be converted to Q under conditions, which preferably do not degrade or react with other portions of the compound of Structure (I). One of skill in the art can derive appropriate protected forms of Q based on the particular Q and desired end use and storage conditions. For example, when Q is SH, a protected form of Q includes a disulfide, which can be reduced to reveal the SH moiety using commonly known techniques and reagents.

Exemplary Q moieties are provided in Table A below.

TABLE A
Exemplary Q Moieties

| Structure | Class |
|---|---|
| 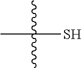 [SMILES: *S] | Sulfhydryl |
| 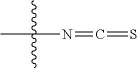 [SMILES: *N=C=S] | Isothiocyanate |
| 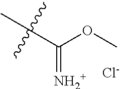 | Imidoester |
| 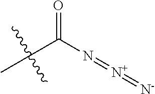 [SMILES: *C(=O)N=[N+]=[N-]] | Acyl Azide |
| 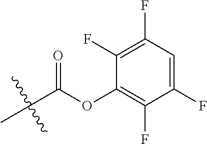 [SMILES: *C(=O)Oc1c(F)c(F)cc(F)c1F] | Activated Ester |
| 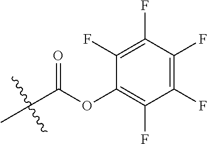 [SMILES: *C(=O)Oc1c(F)c(F)c(F)c(F)c1F] | Activated Ester |
| 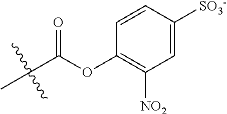 | Activated Ester |
| 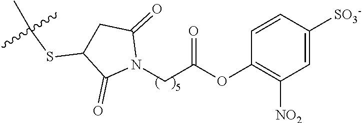 | Activated Ester |
| 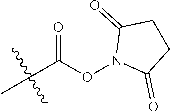 [SMILES: *C(=O)ON1C(=O)CCC1=O] | Activated Ester |
| 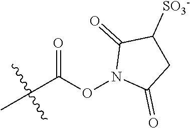 | Activated Ester |
| 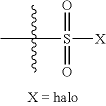 | Sulfonyl halide |
| 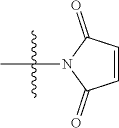 [SMILES: *N1C(=O)C=CC1=O] | Maleimide |
| 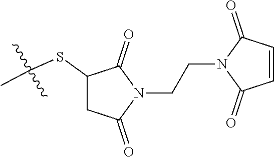 [SMILES: *SC1CC(=O)N(CCN2C(=O)C=CC2=O)C1=O] | Maleimide |
| 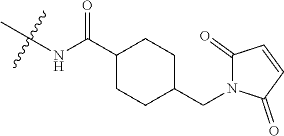 [SMILES: *NC(=O)C1CCC(CN2C(=O)C=CC2=O)CC1] | Maleimide |
| 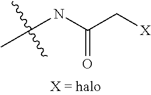 | α-haloimide |
| 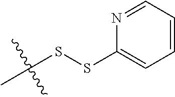 [SMILES: *SSc1ccccn1] | Disulfide |
| 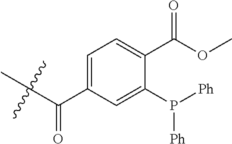 [SMILES: *C(=O)c1ccc(C(=O)OC)c(P(c2ccccc2)c2ccccc2)c1] | Phosphine |
| 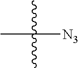 [SMILES: *N=[N+]=[N-]] | Azide |
| 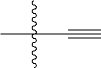 [SMILES: *C#C] | Alkyne |
| 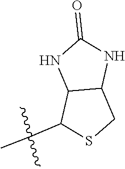 [SMILES: *C1SCC2NC(=O)NC21] | Biotin |
| 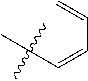 [SMILES: */C=C\C=C] | Diene |
| 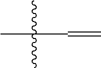 [SMILES: *C=C] | Alkene/dienophile |
| 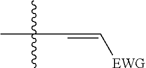 EWG = eletron withdrawing group | Alkene/dienophile |
| —NH₂ | Amino |

It should be noted that in some embodiments, wherein Q is SH, the SH moiety will tend to form disulfide bonds with another sulfhydryl group, for example on another compound of Structure (I). Accordingly, some embodiments include compounds of Structure (I), which are in the form of disulfide dimers, the disulfide bond being derived from SH Q groups.

In some related embodiments, the targeting moiety is an antibody (e.g., brentuximab, gemtuzumab, trastuzumab, inotuzumab, polatuzumab, enfortumab, trastuzumab, sacituzumab, belantamab, or moxetumomab) or cell surface receptor antagonist. In certain embodiments, the antibody or cell surface receptor antagonist is an epidermal growth factor receptor (EGFR) inhibitor, a hepatocyte growth factor receptor (HGFR) inhibitor, an insulin-like growth factor receptor (IGFR) inhibitor, a folate, or a MET inhibitor. In some embodiments, R¹ or R² has one of the following structures:

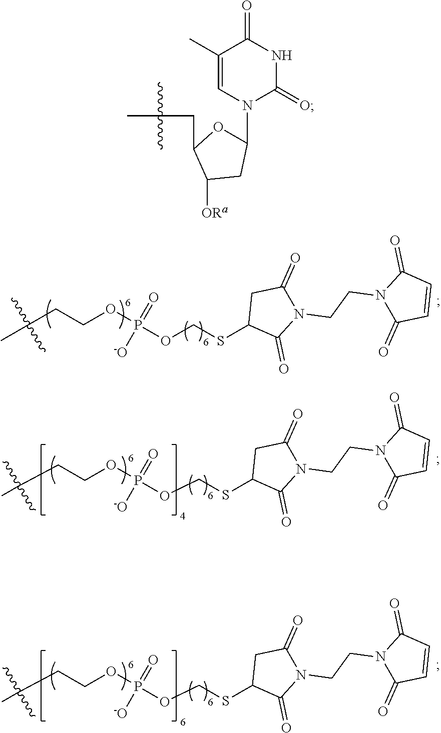

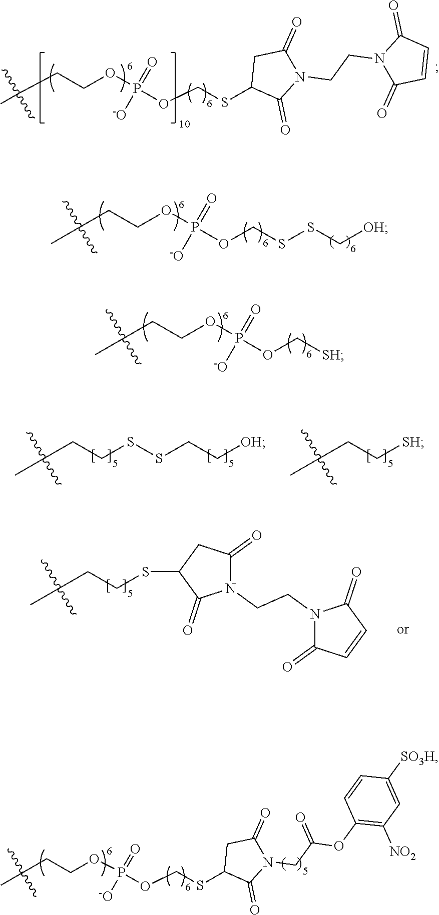

wherein
R^{a} is H or a solid support.

In certain embodiments, R¹ has one of the following structures:

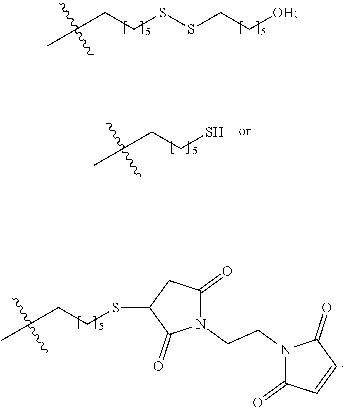

In some embodiments, R² has the following structure:

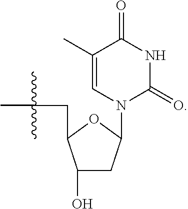 [SMILES: *CC1OC(n2cc(C)c(=O)[nH]c2=O)CC1O]

In certain specific embodiments, at least one occurrence of L¹ is alkylene. In some specific embodiments, at least one occurrence of L¹ is C₁-C₆ alkylene. In some embodiments, at least one occurrence of L¹ is C₁ alkylene. In some other embodiments, at least one occurrence of L¹ is C₂ alkylene. In some embodiments, at least one occurrence of L¹ is C₃ alkylene. In some embodiments, at least one occurrence of L¹ is C₄ alkylene. In some embodiments, at least one occurrence of L¹ is C₅ alkylene. In some embodiments, at least one occurrence of L¹ is C₆ alkylene. In some embodiments, at least one occurrence of L¹ is methylene. In certain embodiments, each occurrence of L¹ is alkylene. In some more specific embodiments, each occurrence of L¹ is C₁-C₆ alkylene. In some other embodiments, each occurrence of L¹ is C₂ alkylene. In some embodiments, each occurrence of L¹ is C₃ alkylene. In some embodiments, each occurrence of L¹ is C₄ alkylene. In some embodiments, each occurrence of L¹ is C₅ alkylene. In some embodiments, each occurrence of L¹ is C₆ alkylene. In some embodiments, each occurrence of L¹ is methylene.

In more specific embodiments, at least one occurrence of L³ is alkylene. In some specific embodiments, at least one occurrence of L³ is C₁-C₆ alkylene. In some other embodiments, at least one occurrence of L³ is C₂ alkylene. In some embodiments, at least one occurrence of L³ is C₃ alkylene. In some embodiments, at least one occurrence of L³ is C₄ alkylene. In some embodiments, at least one occurrence of L³ is C₅ alkylene. In some embodiments, at least one occurrence of L³ is C₆ alkylene. In some specific embodiments, at least one occurrence of L³ is methylene. In certain embodiments, each occurrence of L³ is alkylene. In some embodiments, each occurrence of L³ is C₁-C₆ alkylene. In some other embodiments, each occurrence of L³ is C₂ alkylene. In some embodiments, each occurrence of L³ is C₃ alkylene. In some embodiments, each occurrence of L³ is C₄ alkylene. In some embodiments, each occurrence of L³ is C₅ alkylene. In some embodiments, each occurrence of L³ is C₆ alkylene. In some embodiments, each occurrence of L³ is methylene. In some more specific embodiments, at least one occurrence of L³ is a direct bond. In some embodiments, each occurrence of L³ is a direct bond.

In some specific embodiments, at least one occurrence of L² is heteroalkylene. In certain embodiments, at least one occurrence of L² comprises oxygen. In some embodiments, at least one occurrence of L² has the following structure:

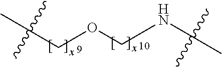

wherein:
x⁹ and x¹⁰ are each independently an integer greater than 0.

In some embodiments, x⁹ is 1, 2, 3, or 4. In certain embodiments, x¹⁰ is 2, 3, 4, or 5. In some specific embodiments, x⁹ is 1 or 2 and x¹⁰ is 2, 3, or 4. In certain specific embodiments, each occurrence of L² is heteroalkylene. In some more specific embodiments, each occurrence of L² comprises oxygen. In certain more specific embodiments, each occurrence of L² has the following structure:

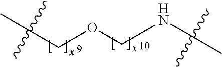

wherein:
x⁹ and x¹⁰ are each independently an integer greater than 0.

In some embodiments, x⁹ is 1, 2, 3, or 4. In certain embodiments, x¹⁰ is 2, 3, 4, or 5. In more specific embodiments, x⁹ is 1 or 2 and x¹⁰ is 2, 3, or 4. In certain other embodiments, at least one occurrence of L² comprises the following structure:

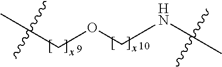

wherein:
x⁹ and x¹⁰ are each independently an integer greater than 0.

In certain embodiments, L² further comprises a physiologically cleavable linker. In more specific embodiments, at least one occurrence of L² comprises an amide bond, an ester bond, a phosphodiester bond, a disulfide bond, a double bond, a triple bond, an ether bond, a hydrazone, an amino acid sequence comprising one or more amino acid residues, a ketone, a diol, a cyano, a nitro, or combinations thereof. In more specific embodiments, at least one occurrence of L² comprises an amino acid sequence recognized by a sortase enzyme or cysteine protease. In certain embodiments, the amino acid sequence is Leu-Pro-X-Thr-Gly, wherein X is any amino acid residue. In more specific embodiments, at least one occurrence of L² comprises one of the following structures:

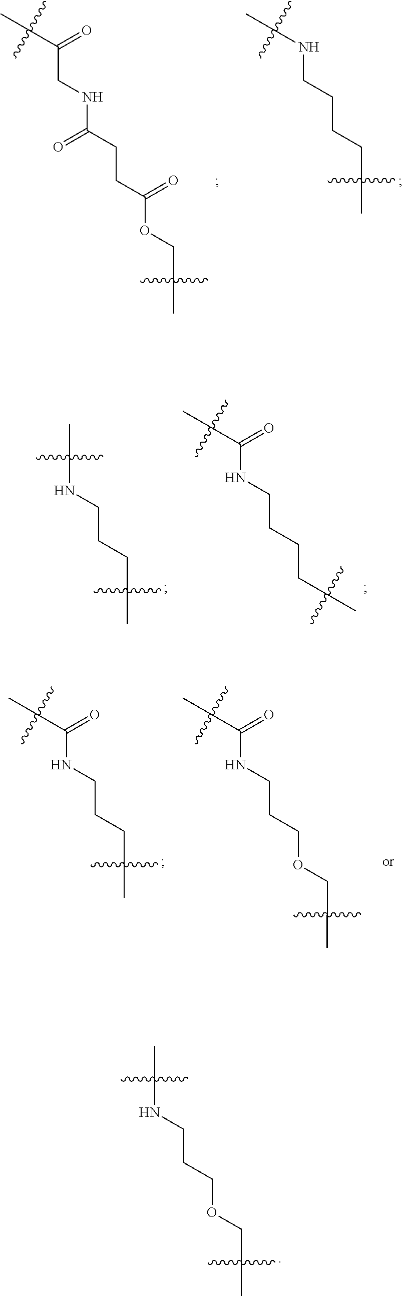

In certain embodiments, each occurrence of L² comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof. In some more specific embodiments, each occurrence of L² comprises one of the following structures:

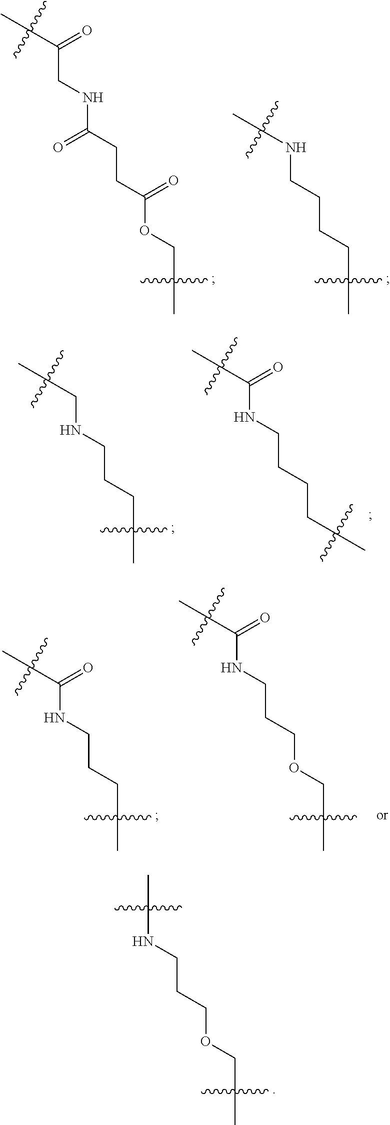

In some more specific embodiments, at least one occurrence of L² comprises one or more amino acid residues. In certain specific embodiments, at least one occurrence of L² comprises one or more amino acid residues selected from the group consisting of alanine, valine, and combinations thereof. In certain embodiments, at least one occurrence of L² comprises one of the following structures:

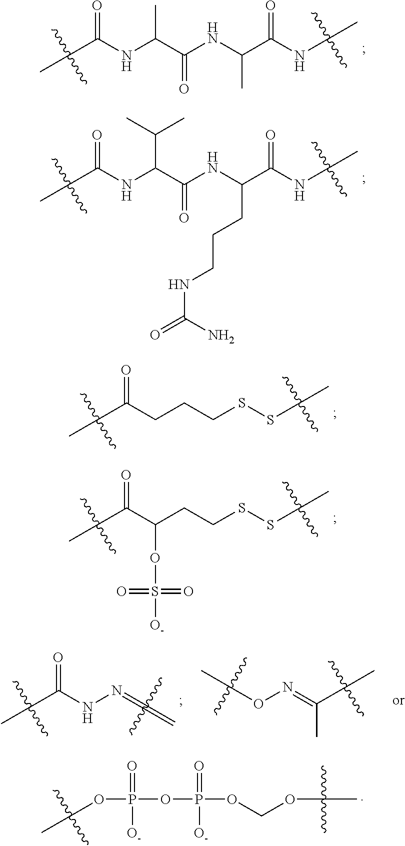

In some embodiments, each occurrence of L² comprises one or more amino acid residues. In certain embodiments, each occurrence of L² comprises one or more amino acid residues selected from the group consisting of alanine, valine, and combinations thereof. In some more specific embodiments, each occurrence of L² comprises one of the following structures:

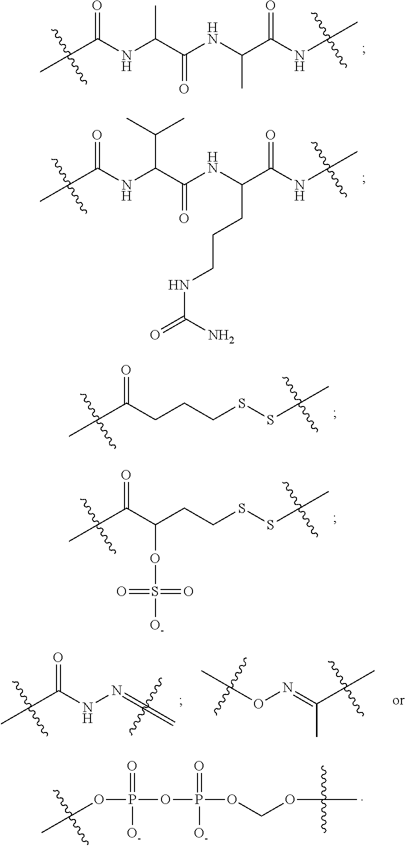

In more specific embodiments, at least one occurrence of L² has one of the following structures:

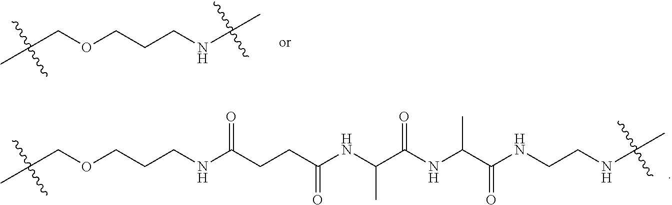 [SMILES: *COCCCN* ; *COCCCNC(=O)CCC(=O)NC(C)C(=O)NC(C)C(=O)NCCN*]

In some specific embodiments, each occurrence of L² has one of the following structures:

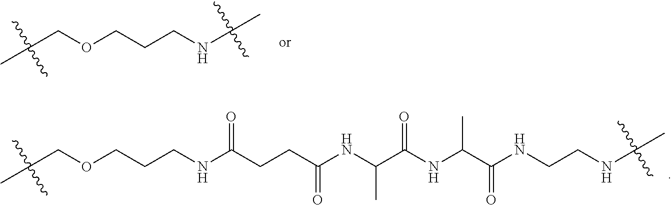 [SMILES: *COCCCN* ; *COCCCNC(=O)CCC(=O)NC(C)C(=O)NC(C)C(=O)NCCN*]

In some embodiments, at least one occurrence of M is an alkylating agent, an antimetabolite, a microtubule inhibitor, a topoisomerase inhibitor, or a cytotoxic antibiotic. In more specific embodiments, each occurrence of M is an alkylating agent, an antimetabolite, a microtubule inhibitor, a topoisomerase inhibitor, or a cytotoxic antibiotic. In some embodiments, at least one occurrence of M is an alkylating agent, an antimetabolite, a microtubule inhibitor, or a topoisomerase inhibitor. In more specific embodiments, each occurrence of M is an alkylating agent, an antimetabolite, a microtubule inhibitor, or a topoisomerase inhibitor. In certain embodiments, at least one occurrence of M is a nitrogen mustard, a nitrosourea, a tetrazine, an aziridine, a cisplatin or cisplatin derivative, or a non-classical alkylating agent. In more specific embodiments, at least one occurrence of M is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine. In some embodiments, at least one occurrence of M is an anti-folate, a fluoropyrimidines, a deoxynucleoside analogue, or a thiopurine. In certain embodiments, at least one occurrence of M is methotrexate, pemetrexed, fluorouracil, capecitabine, cytarabine, gemcitabine, decitabine, azacitidine, fludarabine, nelarabine, cladribine, clofarabine, pentostatin, thioguanine, and mercaptopurine. In some specific embodiments, at least one occurrence of M is an auristatin, a Vinca alkaloid, or a taxane. In certain specific embodiments, at least one occurrence of M is auristatin F, auristatin E, vincristine, vinblastine, vinorelbine, vindesine, vinflunine, paclitaxel, docetaxel, etoposide, or teniposide. In some more specific embodiments, at least one occurrence of M is irinotecan, SN 38, topotecan, camptothecin, doxorubicin, mitoxantrone, teniposide. novobiocin, merbarone, or aclarubicin. In certain more specific embodiments, at least one occurrence of M is an anthracycline or a bleomycin. In some embodiments, at least one occurrence of M is doxorubicin, daunorubicin, epirubicin, idarubicin, pirarubicin, aclarubicin, or mitoxantrone. In some embodiments, at least one occurrence of M is auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan. In some embodiments, each occurrence of M is auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

In certain embodiments, at least one occurrence of M has the following structure:

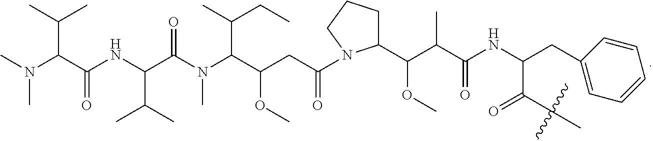 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

In some specific embodiments, each occurrence of M has the following structure:

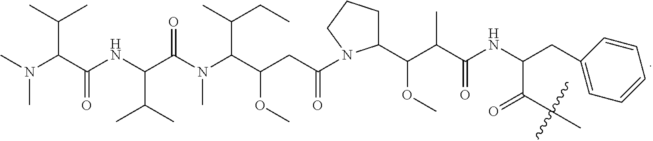 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

In certain embodiments, at least one occurrence of M has the following structure:

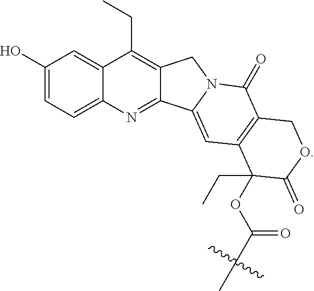 [SMILES: *C(=O)OC1(CC)C(=O)OCc2c1cc1n(c2=O)Cc2c-1nc1ccc(O)cc1c2CC]

In certain embodiments, each occurrence of M has the following structure:

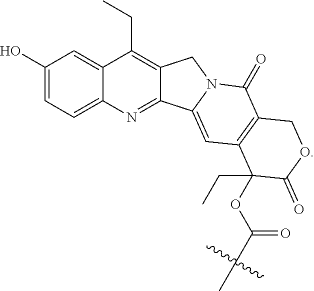 [SMILES: *C(=O)OC1(CC)C(=O)OCc2c1cc1n(c2=O)Cc2c-1nc1ccc(O)cc1c2CC]

In certain specific embodiments, at least one occurrence of -L²-M has one of the following structures:

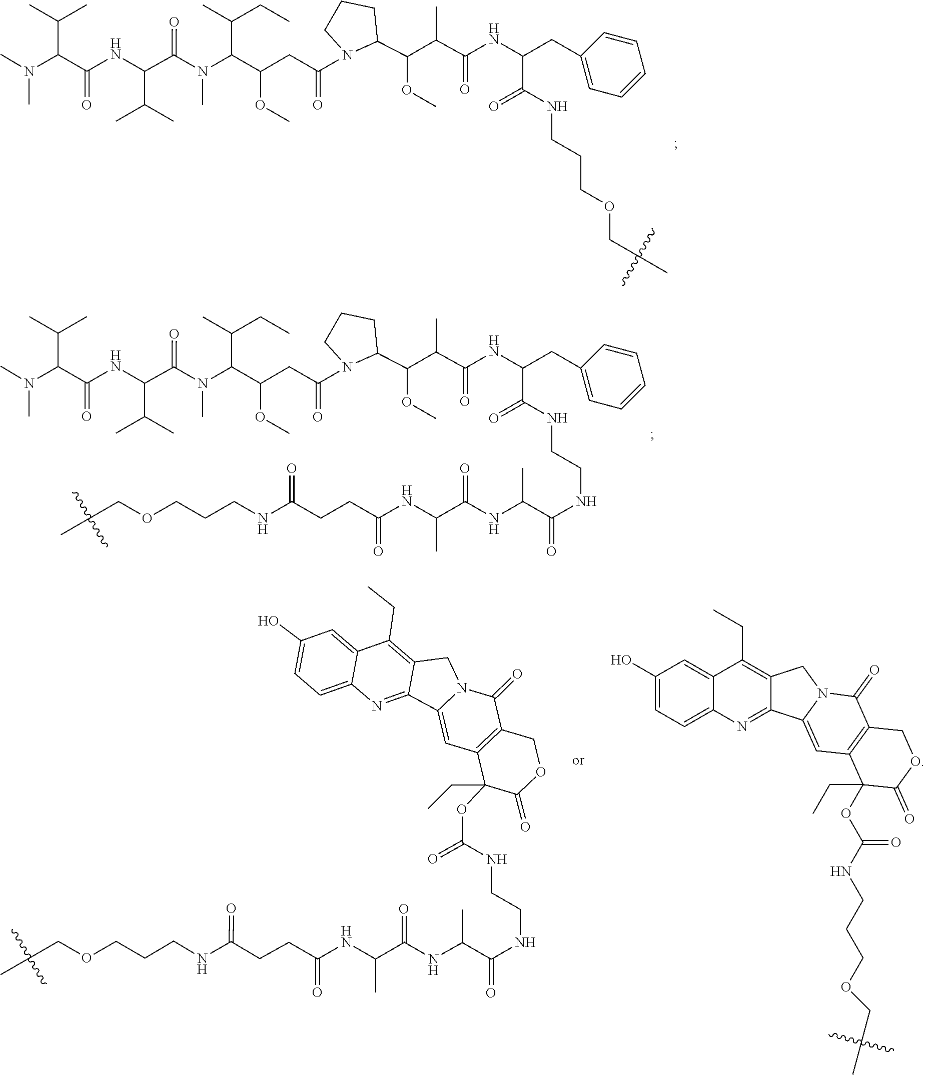

In some more specific embodiments, each occurrence of -L²-M has one of the following structures:

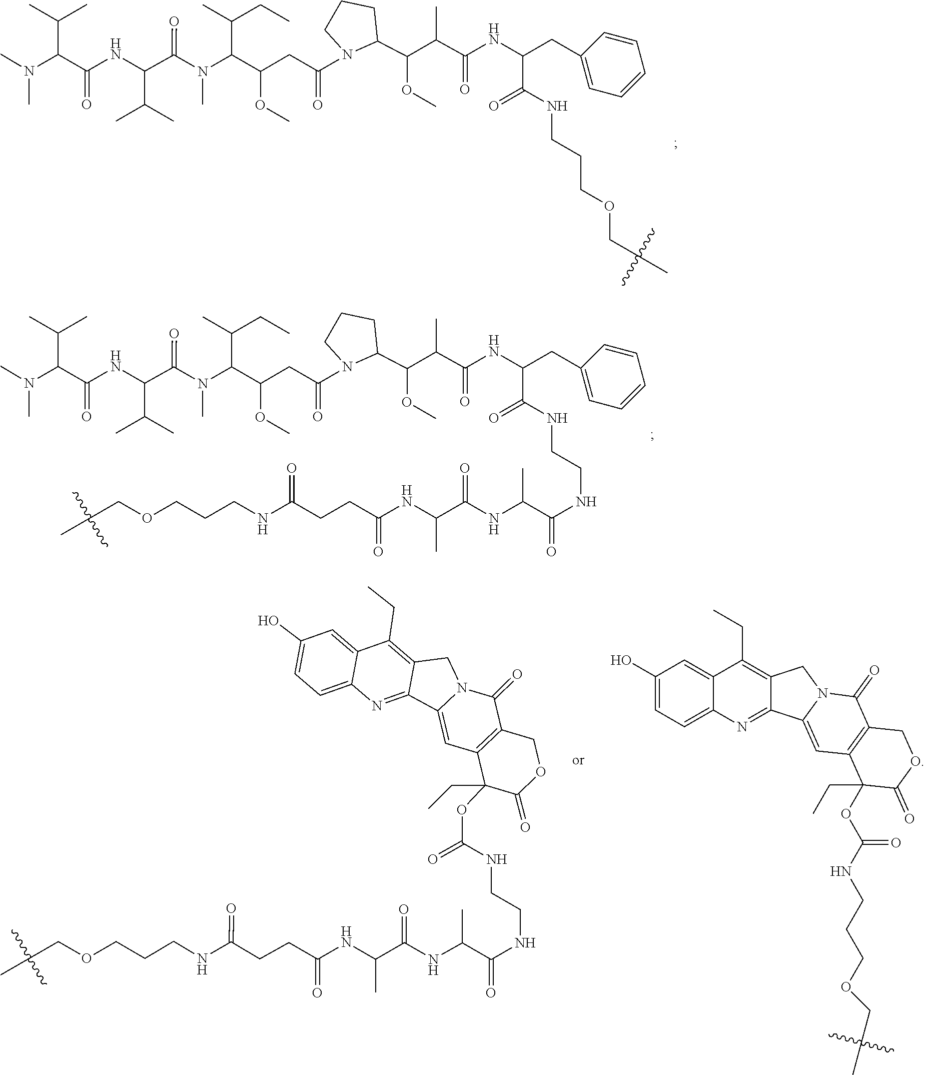

In some specific embodiments, n is 1, 2, 3, 4, 5, or 6. In some more specific embodiments, n is 1, 2, 3, or 4.

In some more specific embodiments, n is an integer greater than or equal to 2 and at least one occurrence of -L²-M has the following structure:

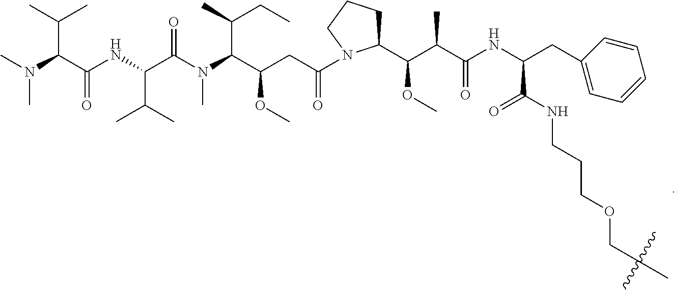 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

In certain more specific embodiments, each occurrence of -L²-M has the following structure:

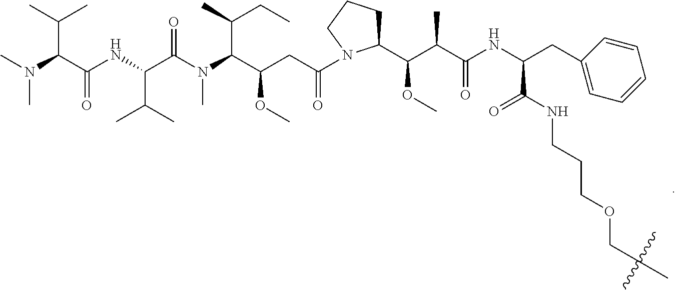 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

In some embodiments, n is an integer greater than or equal to 2 and at least one occurrence of -L²-M has the following structure:

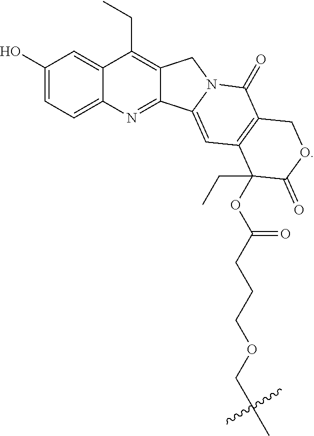 [SMILES: *COCCCC(=O)OC1(CC)C(=O)OCc2c1cc1n(c2=O)Cc2c-1nc1ccc(O)cc1c2CC]

In some more specific embodiments, each occurrence of -L²-M has the following structure:

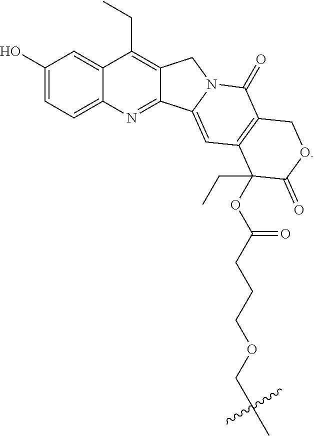 [SMILES: *COCCCC(=O)OC1(CC)C(=O)OCc2c1cc1n(c2=O)Cc2c-1nc1ccc(O)cc1c2CC]

In certain embodiments, n is an integer greater than or equal to 2 and at least one occurrence of -L²-M has the following structure:

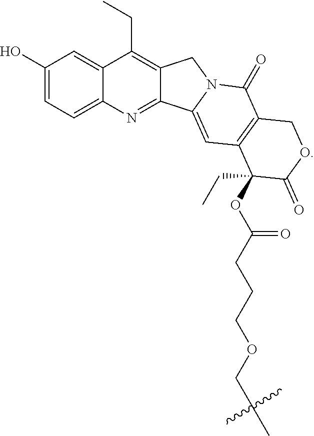 [SMILES: *COCCCC(=O)O[C@]1(CC)C(=O)OCc2c1cc1n(c2=O)Cc2c-1nc1ccc(O)cc1c2CC]

In certain embodiments, each occurrence of -L²-M has the following structure:

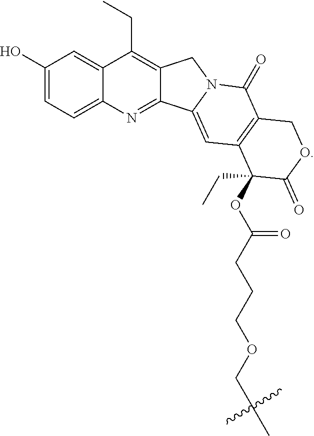 [SMILES: *COCCCC(=O)O[C@]1(CC)C(=O)OCc2c1cc1n(c2=O)Cc2c-1nc1ccc(O)cc1c2CC]

In some more specific embodiments, n is 2, 3, 4, 5, or 6 and at least one e occurrence of -L²-M has the following structure:

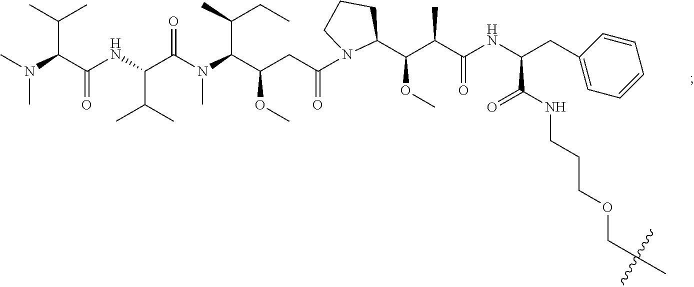 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

and
at least one other occurrence of -L²-M has the following structure:

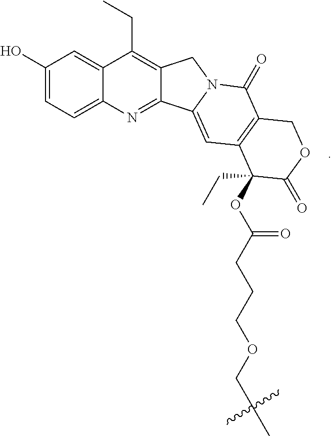 [SMILES: *COCCCC(=O)O[C@]1(CC)C(=O)OCc2c1cc1n(c2=O)Cc2c-1nc1ccc(O)cc1c2CC]

In some embodiments, n is 3 and two occurrences of -L²-M have the following structure:

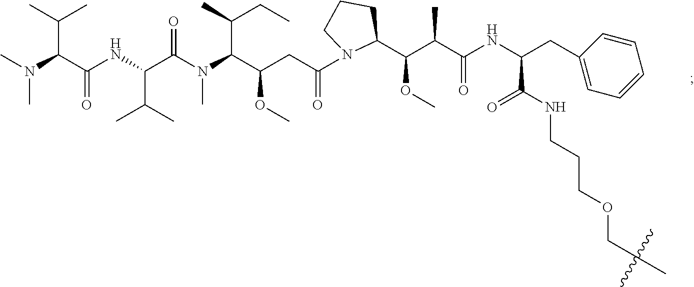 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

and
the other occurrence of -L²-M has the following structure:

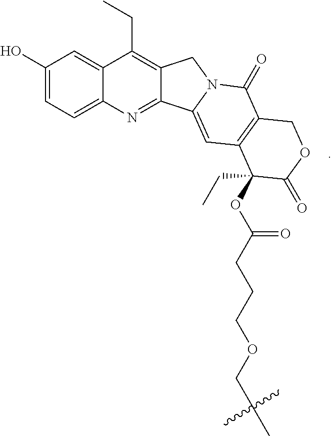 [SMILES: *COCCCC(=O)O[C@]1(CC)C(=O)OCc2c1cc1n(c2=O)Cc2c-1nc1ccc(O)cc1c2CC]

In some embodiments, n is 3 and two occurrences of -L²-M have the following structure:

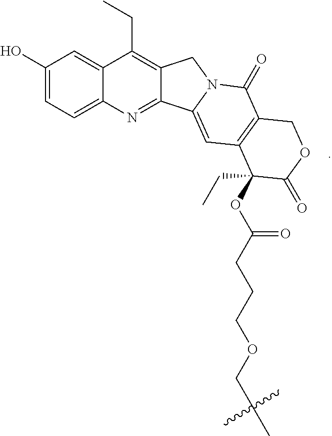 [SMILES: *COCCCC(=O)O[C@]1(CC)C(=O)OCc2c1cc1n(c2=O)Cc2c-1nc1ccc(O)cc1c2CC]

and
the other occurrence of -L²-M has the following structure:

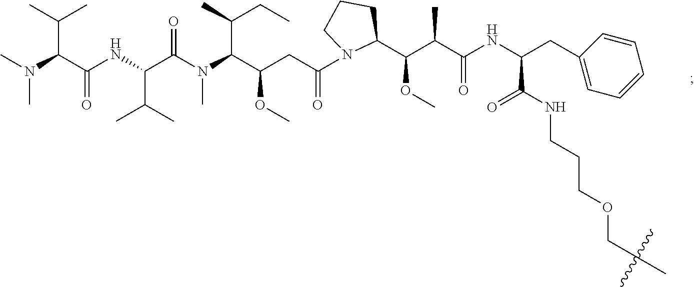 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

In some embodiments, n is 4 and two occurrences of -L²-M have the following structure:

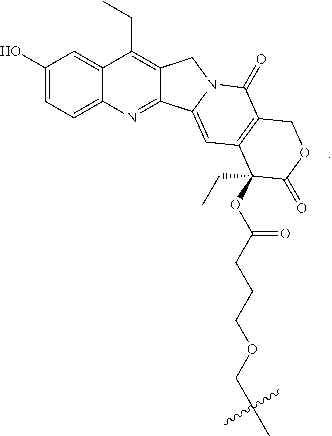 [SMILES: *COCCCC(=O)O[C@]1(CC)C(=O)OCc2c1cc1n(c2=O)Cc2c-1nc1ccc(O)cc1c2CC]

and
the other two occurrence of -L²-M has the following structure:

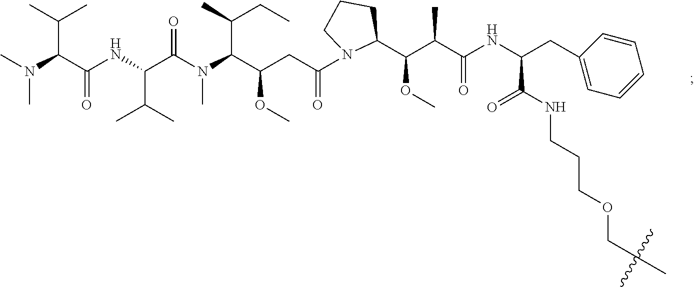 [SMILES: *COCCCNC(=O)[C@H](Cc1ccccc1)NC(=O)[C@H](C)[C@@H](OC)[C@@H]1CCCN1C(=O)C[C@@H](OC)[C@H]([C@@H](C)CC)N(C)C(=O)[C@@H](NC(=O)[C@H](C(C)C)N(C)C)C(C)C]

In some embodiments, M has the following structure:

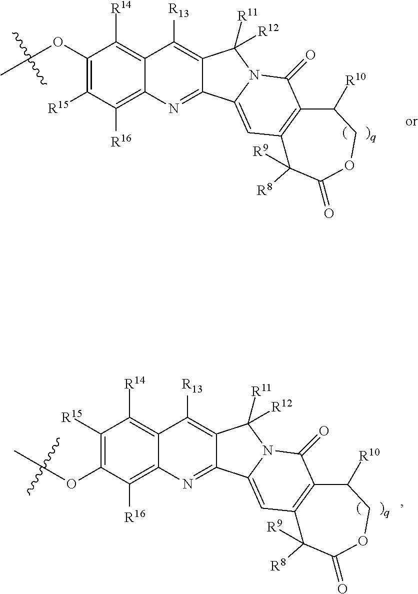

wherein:
R⁸ is, at each occurrence, independently H or OH;
R⁹, R¹⁰, R¹¹, and R¹² are, at each occurrence, independently H, linear or branched alkyl, linear or branched alkyaryl, hydroxyalkyl, or aryl;
R¹³, R¹⁴, R¹⁵, and R¹⁶ are, at each occurrence, independently H, linear or branched alkyl, linear or branched alkyaryl, carbonyl, alkoxy, aryloxy, hydroxyalkyl, nitro, cyano, or aminoalkoxy; and
q is 0 or 1.

In some more specific embodiments, at least one occurrence of M has the following structure:

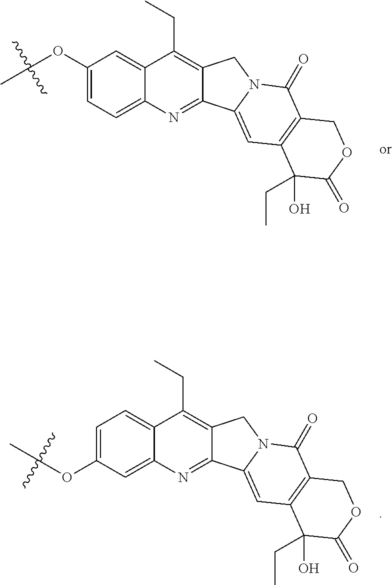 [SMILES: *Oc1ccc2nc3c(c(CC)c2c1)Cn1c-3cc2c(c1=O)COC(=O)C2(O)CC ; *Oc1ccc2c(CC)c3c(nc2c1)-c1cc2c(c(=O)n1C3)COC(=O)C2(O)CC]

In certain embodiment, each occurrence of M has the following structure:

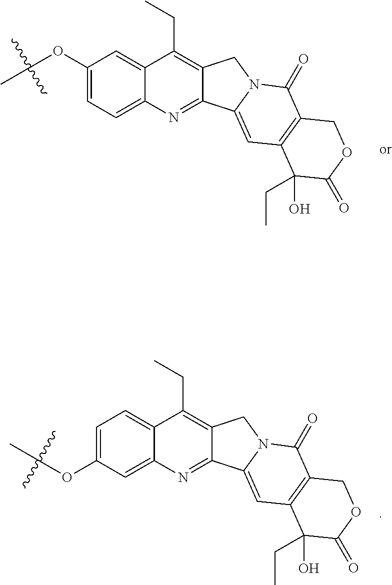 [SMILES: *Oc1ccc2nc3c(c(CC)c2c1)Cn1c-3cc2c(c1=O)COC(=O)C2(O)CC ; *Oc1ccc2c(CC)c3c(nc2c1)-c1cc2c(c(=O)n1C3)COC(=O)C2(O)CC]

In some embodiments, at least one occurrence of -L²-M has one of the following structures:

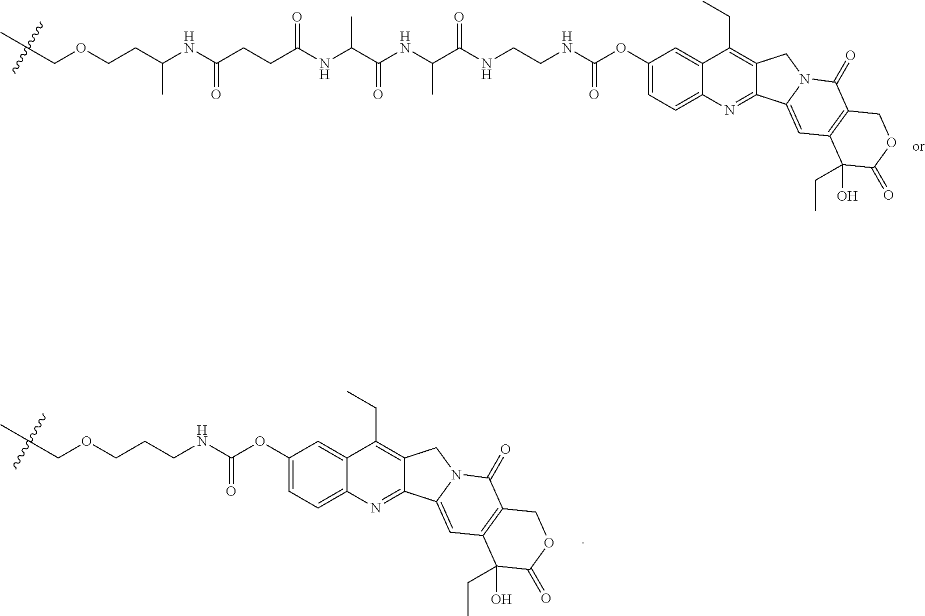 [SMILES: *COCCC(C)NC(=O)CCC(=O)NC(C)C(=O)NC(C)C(=O)NCCNC(=O)Oc1ccc2nc3c(c(CC)c2c1)Cn1c-3cc2c(c1=O)COC(=O)C2(O)CC ; *COCCCNC(=O)Oc1ccc2nc3c(c(CC)c2c1)Cn1c-3cc2c(c1=O)COC(=O)C2(O)CC]

Further in some embodiments, each occurrence of -L²-M has one of the following structures:

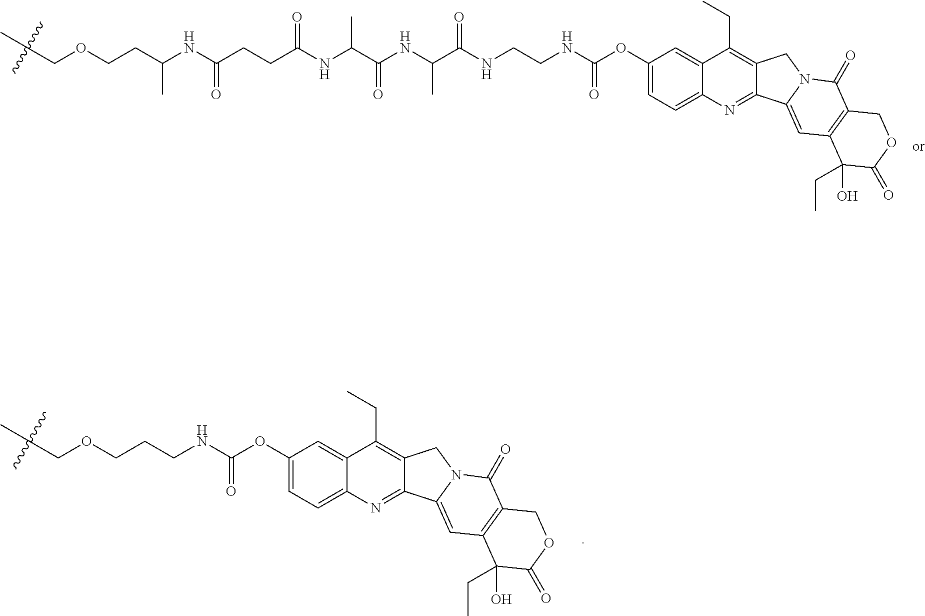 [SMILES: *COCCC(C)NC(=O)CCC(=O)NC(C)C(=O)NC(C)C(=O)NCCNC(=O)Oc1ccc2nc3c(c(CC)c2c1)Cn1c-3cc2c(c1=O)COC(=O)C2(O)CC ; *COCCCNC(=O)Oc1ccc2nc3c(c(CC)c2c1)Cn1c-3cc2c(c1=O)COC(=O)C2(O)CC]

In some embodiments, at least one occurrence of -L²-M has one of the following structures:

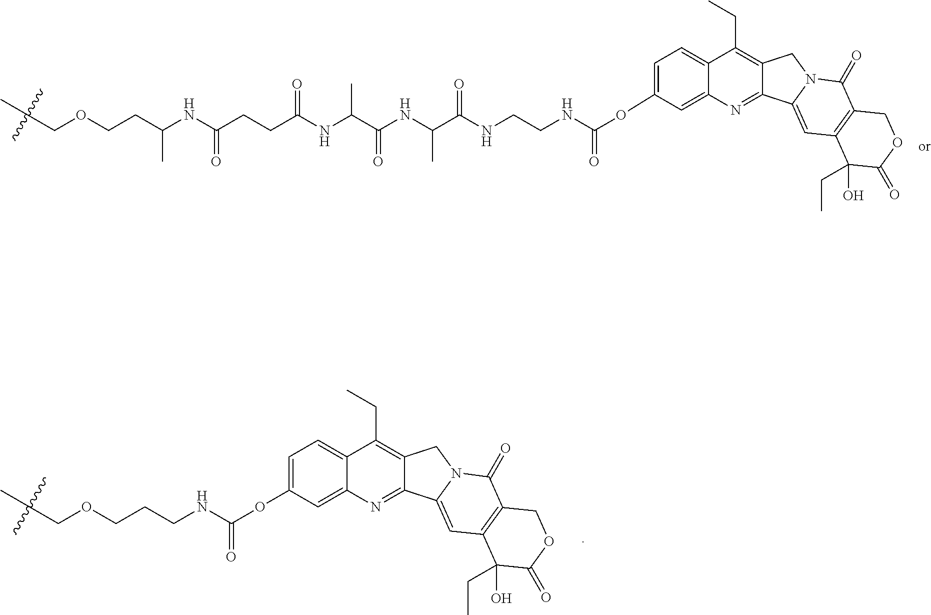 [SMILES: *COCCC(C)NC(=O)CCC(=O)NC(C)C(=O)NC(C)C(=O)NCCNC(=O)Oc1ccc2c(CC)c3c(nc2c1)-c1cc2c(c(=O)n1C3)COC(=O)C2(O)CC ; *COCCCNC(=O)Oc1ccc2c(CC)c3c(nc2c1)-c1cc2c(c(=O)n1C3)COC(=O)C2(O)CC]

In some more embodiments, each occurrence of -L²-M has one of the following structures:

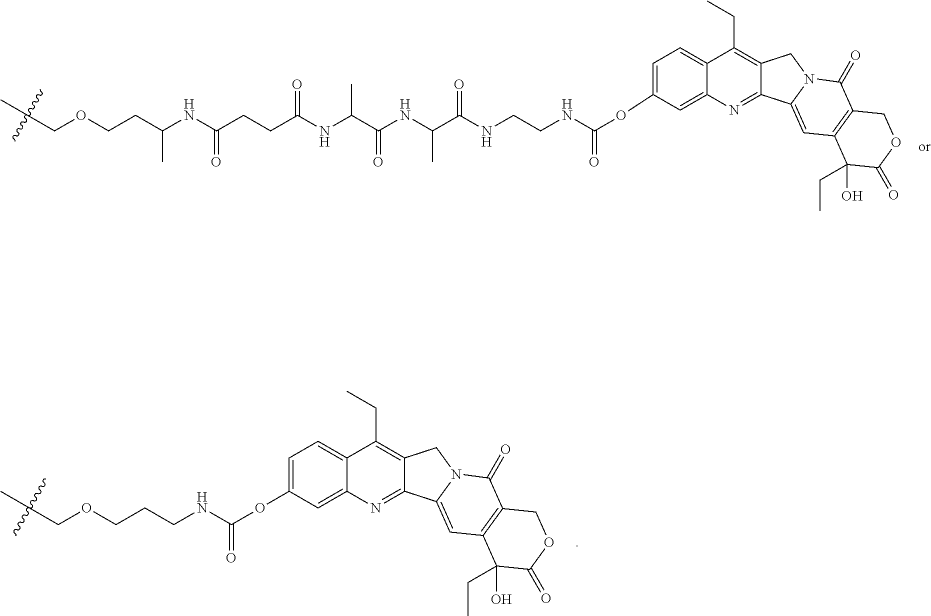 [SMILES: *COCCC(C)NC(=O)CCC(=O)NC(C)C(=O)NC(C)C(=O)NCCNC(=O)Oc1ccc2c(CC)c3c(nc2c1)-c1cc2c(c(=O)n1C3)COC(=O)C2(O)CC ; *COCCCNC(=O)Oc1ccc2c(CC)c3c(nc2c1)-c1cc2c(c(=O)n1C3)COC(=O)C2(O)CC]

TABLE 1
Representative compounds of Structure (I)

| Compound | | |
|---|---|---|
| No. | Structure | [M + H]⁺ |
| I-1 | 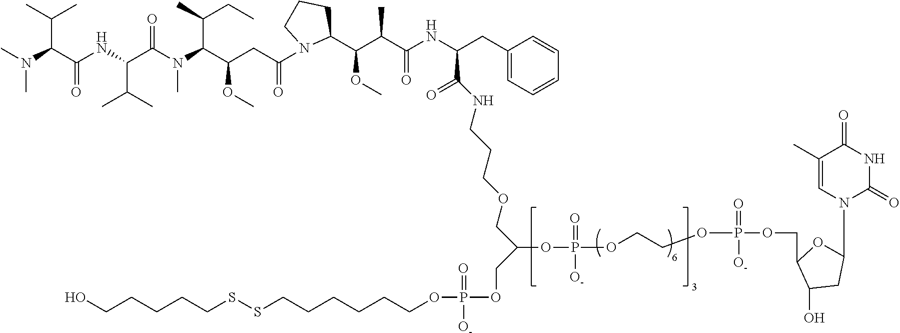 | 2521.6 |
| I-2 | 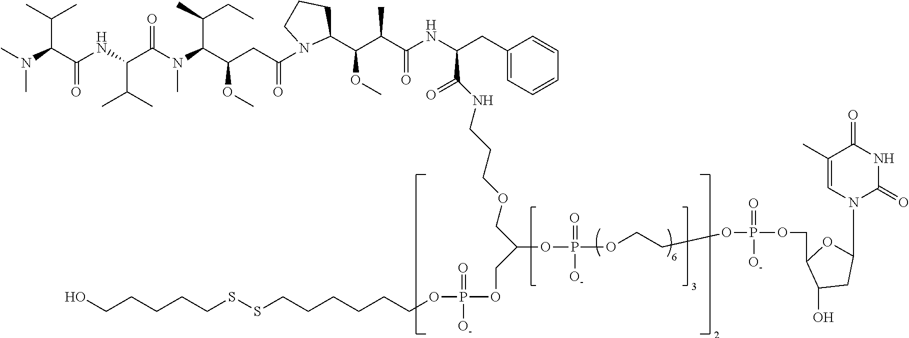 | 2623.4 |
| I-3 | 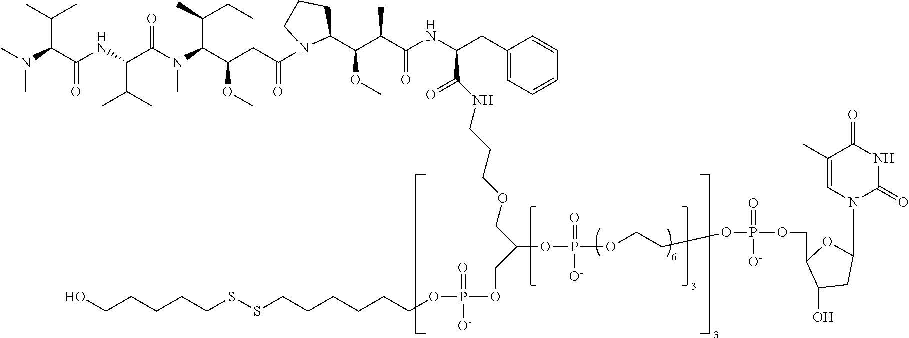 | 2838.2 |
| I-4 | 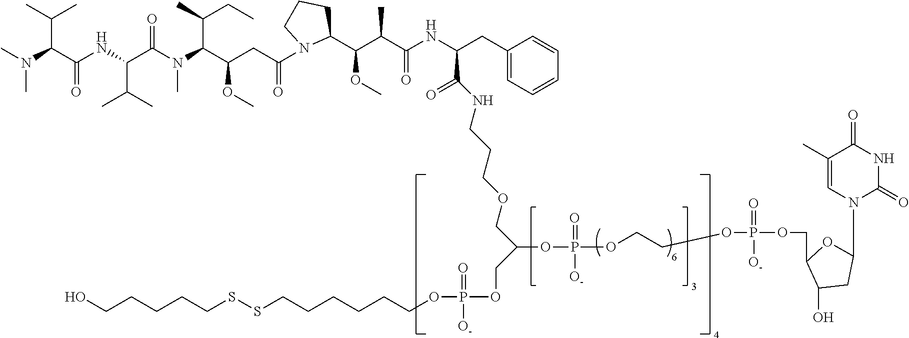 | 3260.4 |
| I-5 | 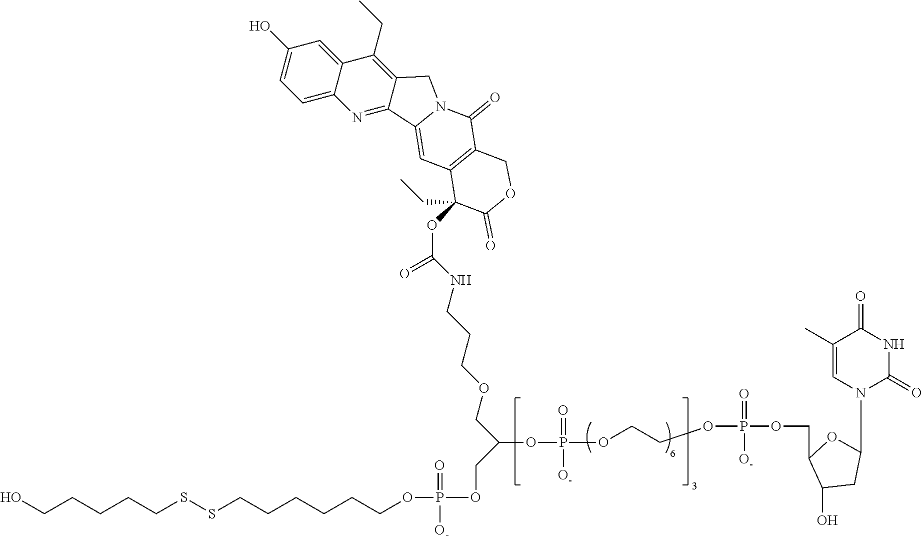 | — |
| I-6 | 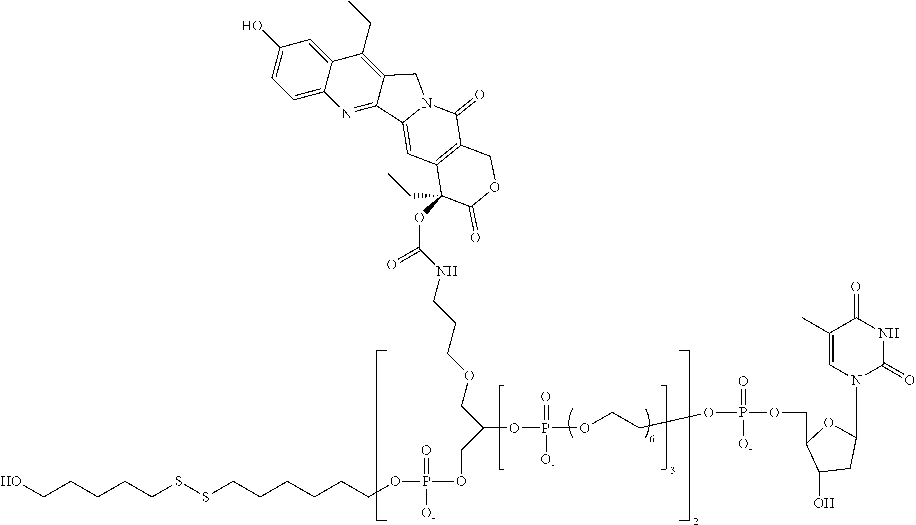 | — |
| I-7 | 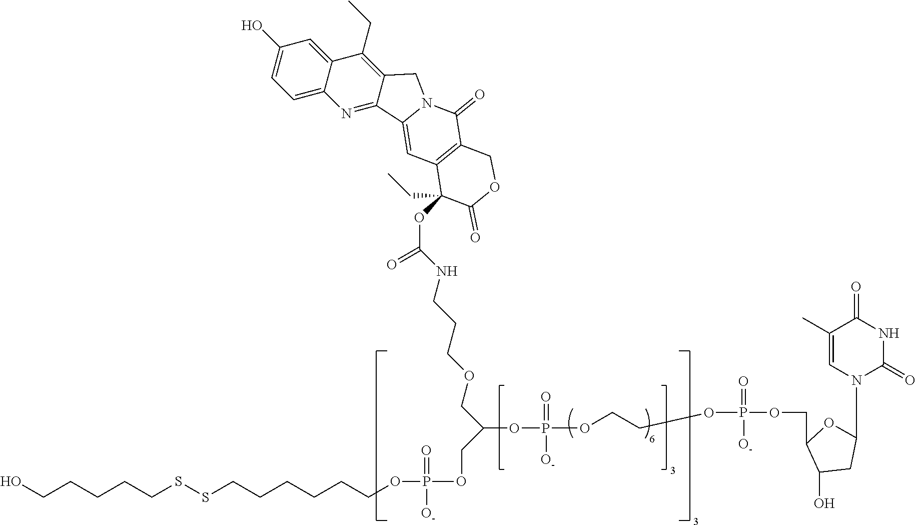 | — |
| I-8 | 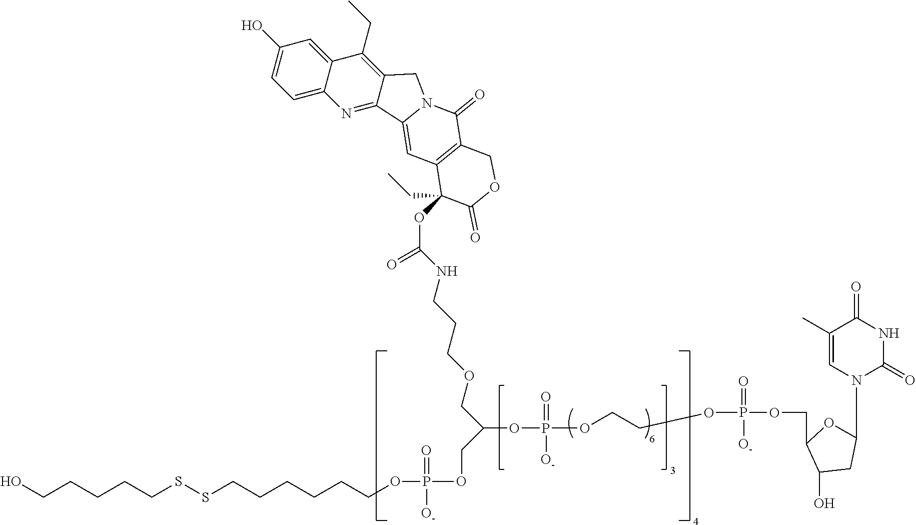 | — |
| I-9 | 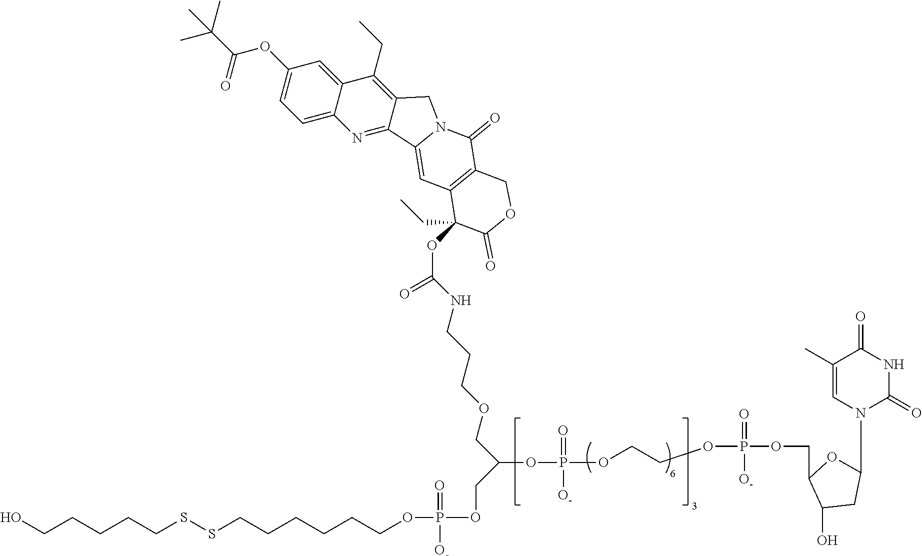 | — |
| I-10 | 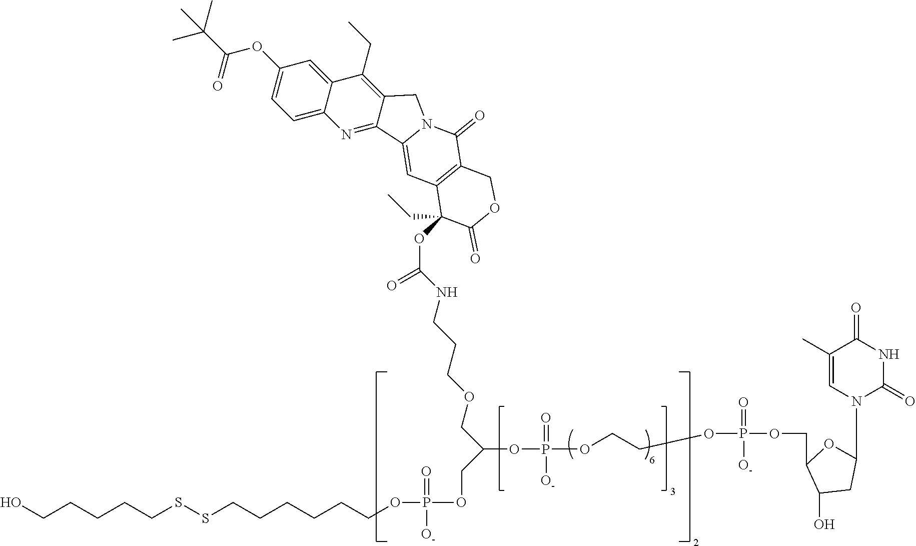 | — |
| I-11 | 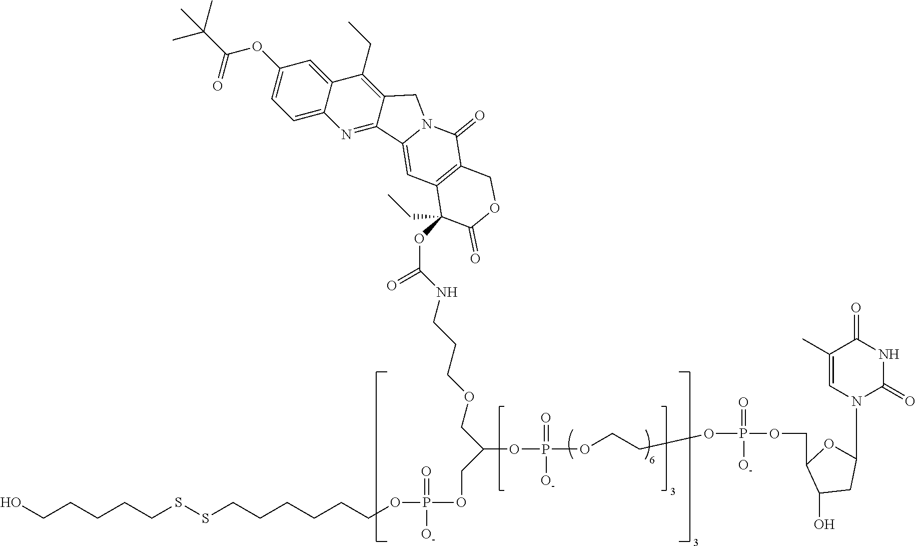 | — |
| I-12 | 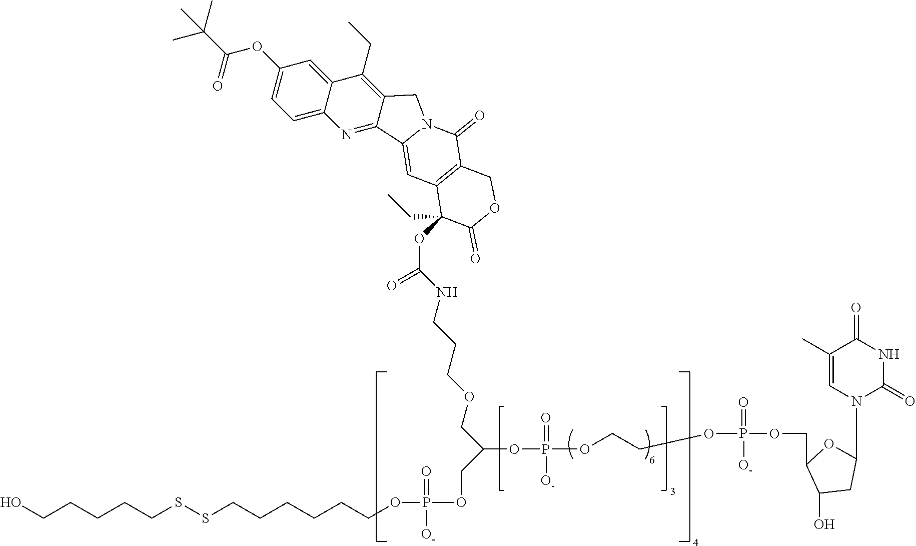 | — |
| I-13 | 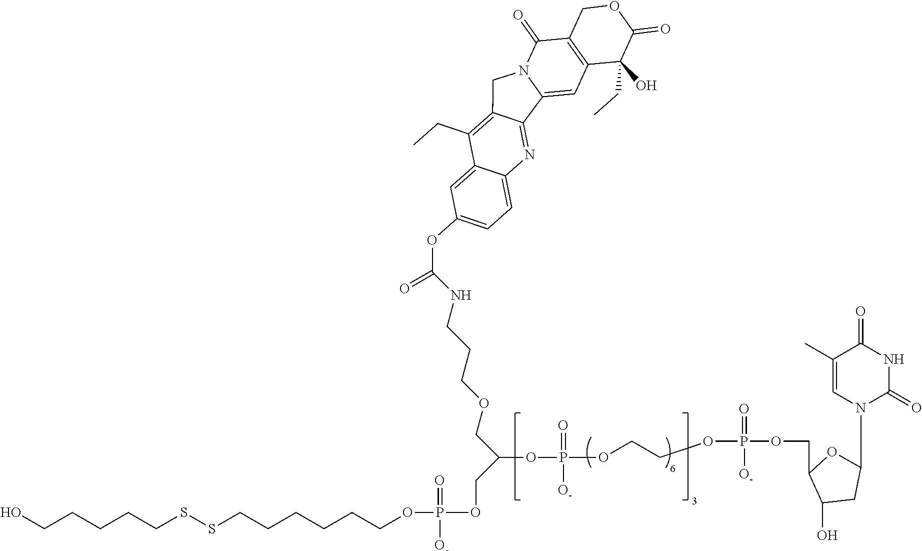 | |
| I-14 | 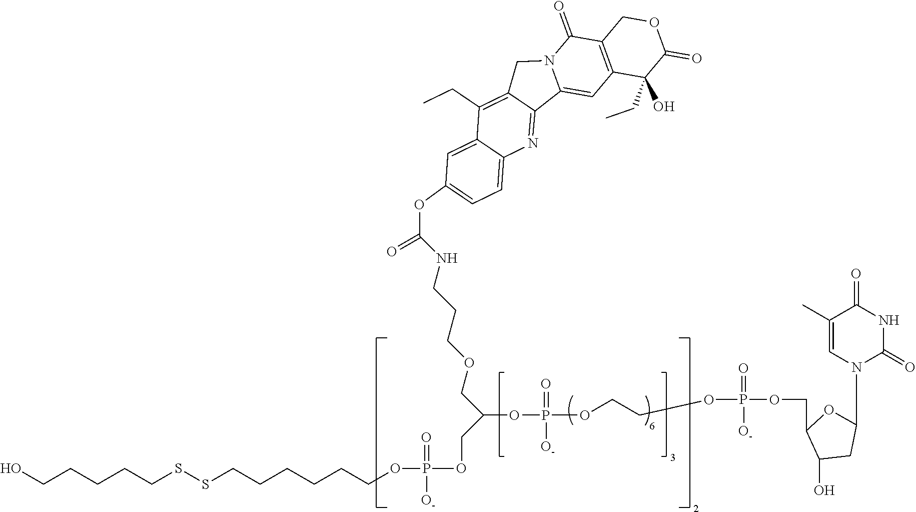 | |
| I-15 | 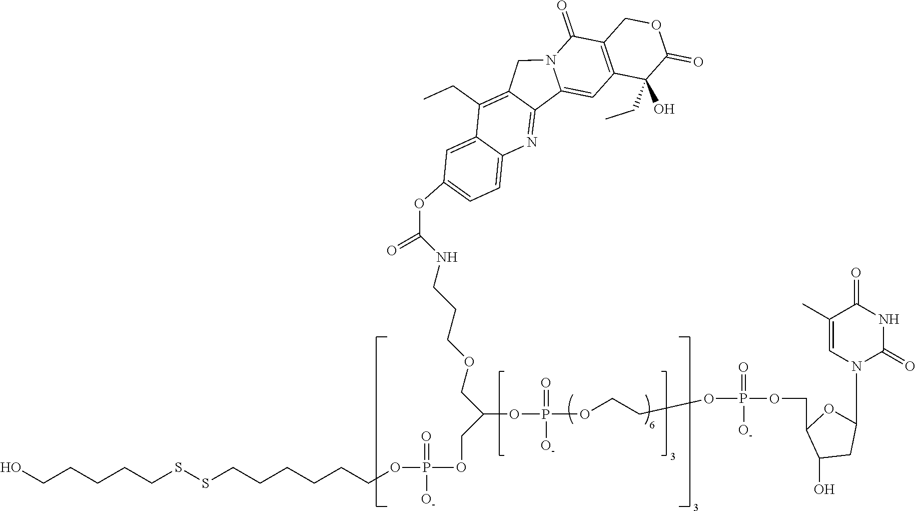 | |
| I-16 | 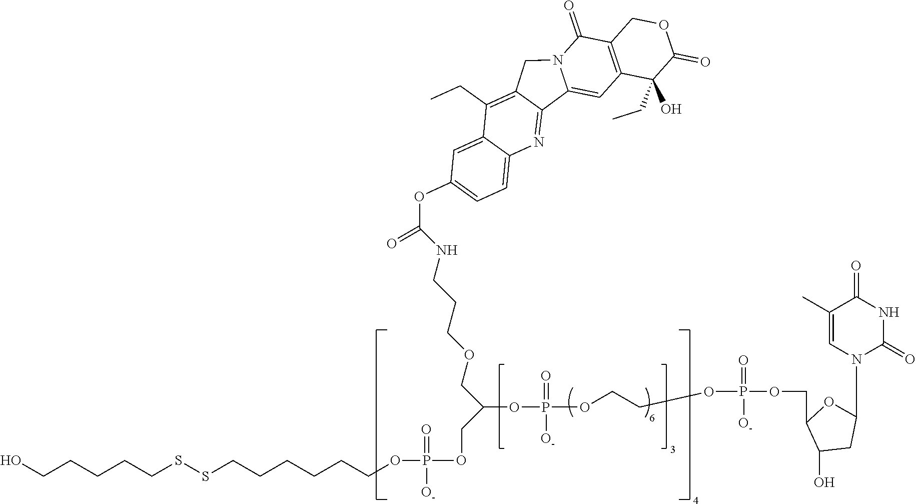 | |
| I-17 | 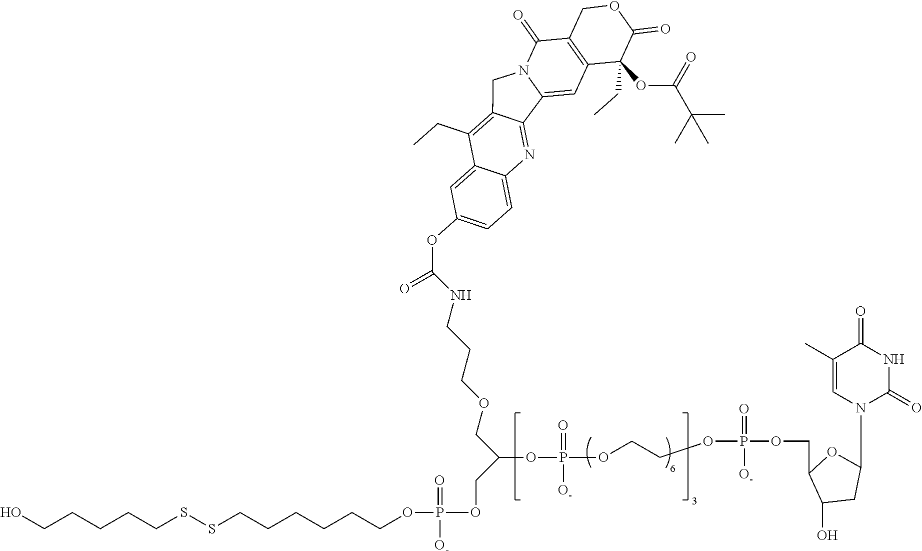 | |
| I-18 | 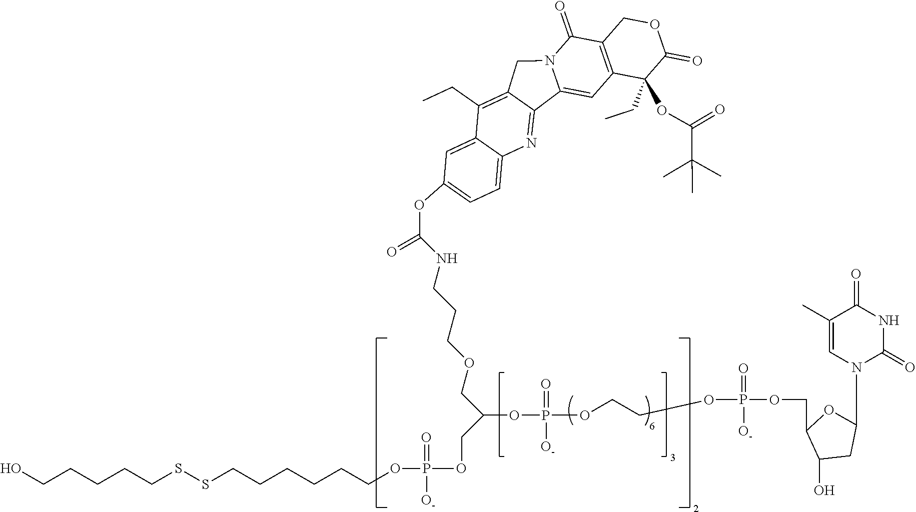 | |
| I-19 | 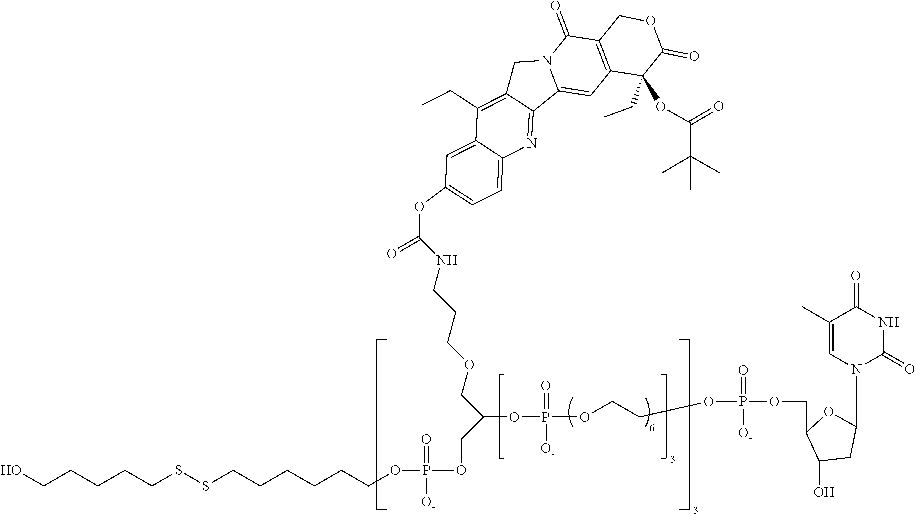 | |
| I-20 | 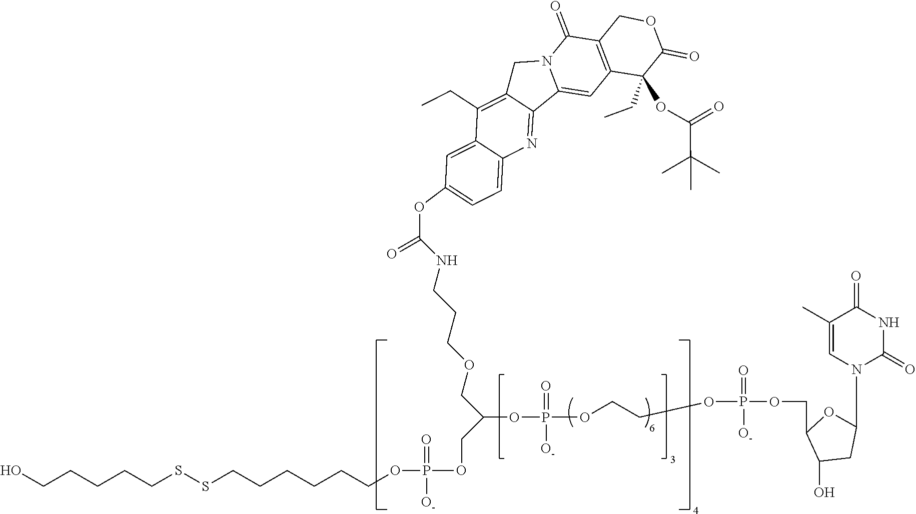 | |
| I-21 | 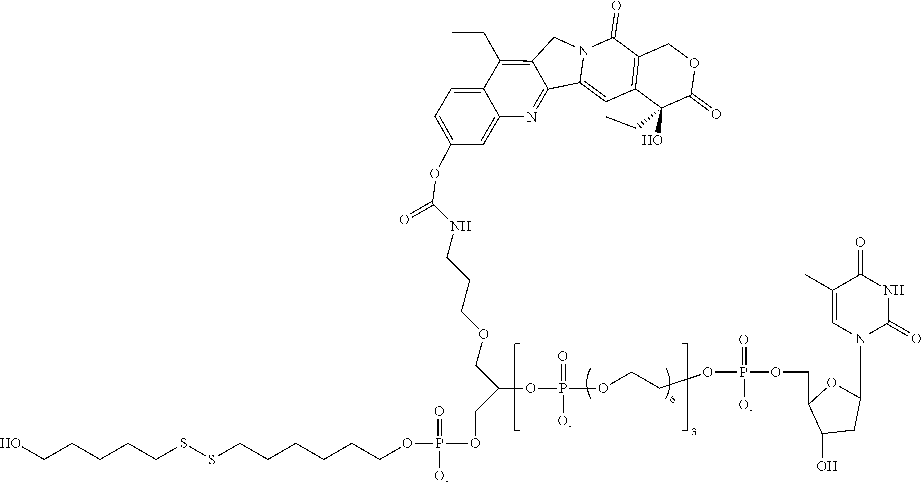 | |
| I-22 | 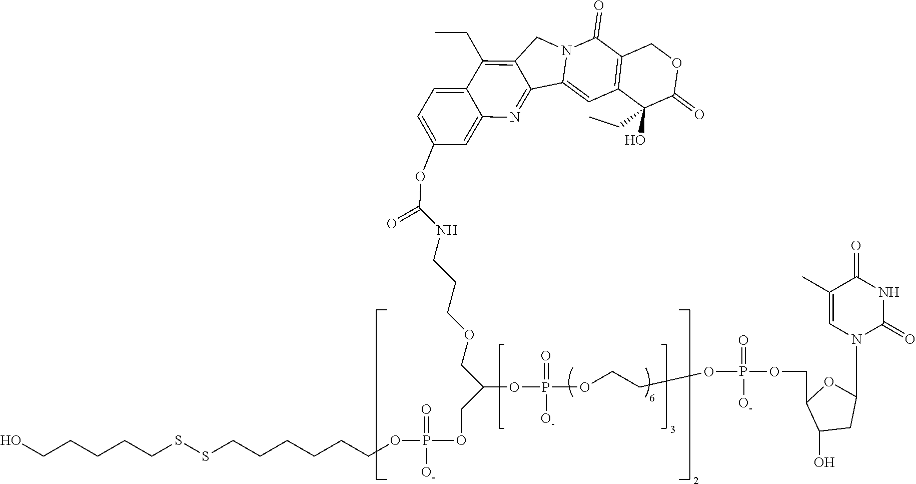 | |
| I-23 | 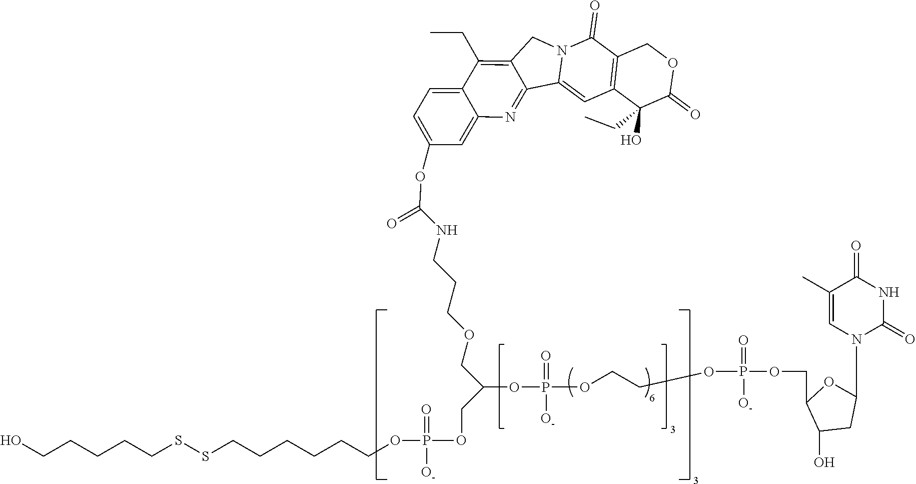 | |
| I-24 | 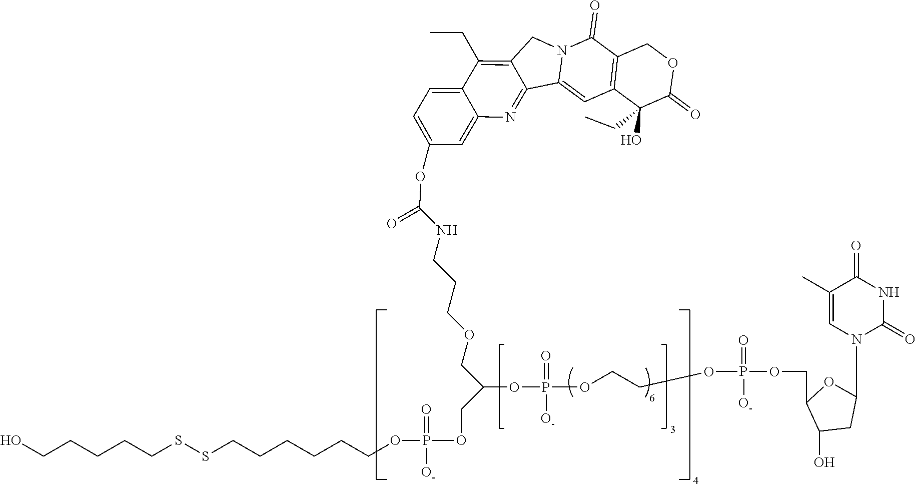 | |
| I-25 | 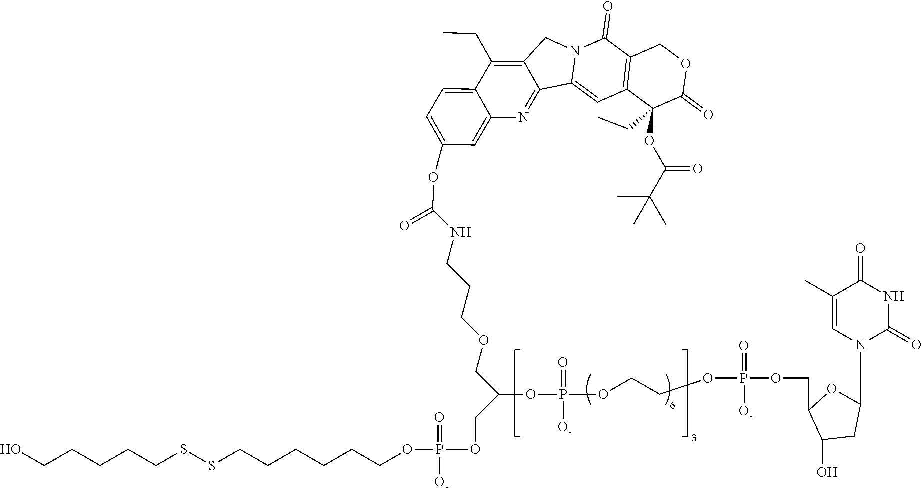 | |
| I-26 | 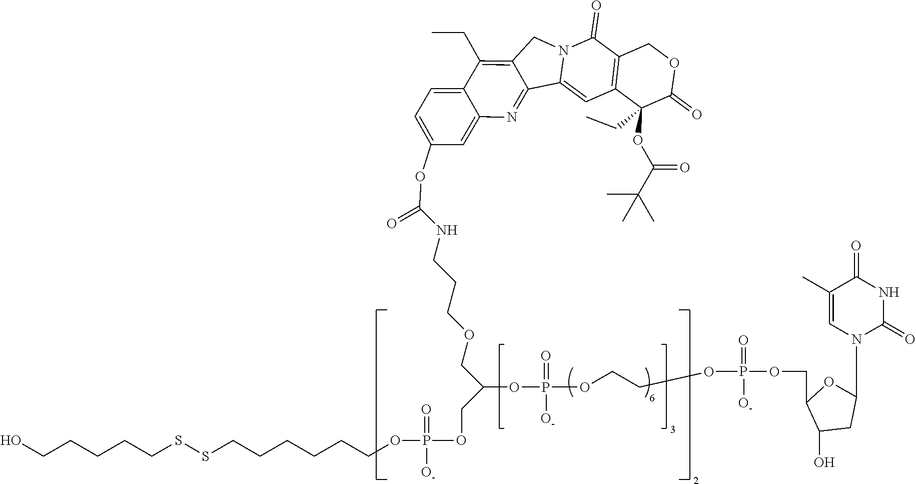 | |
| I-27 | 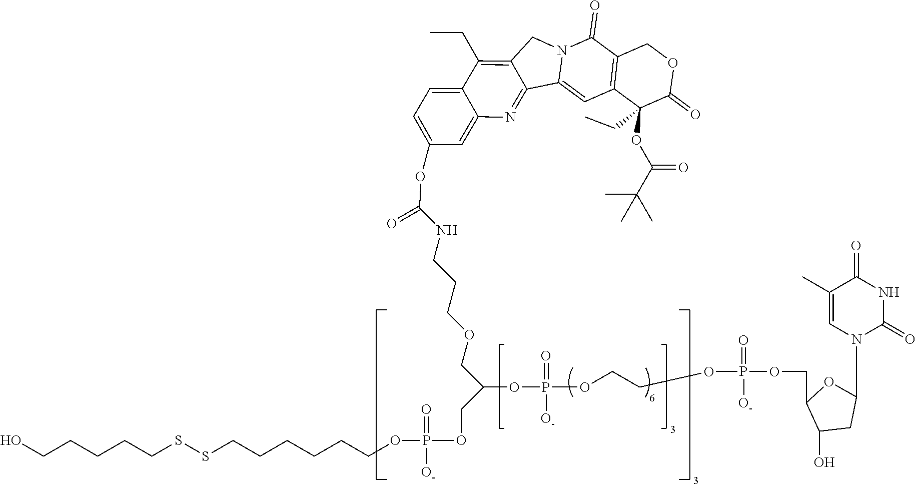 | |
| I-28 | 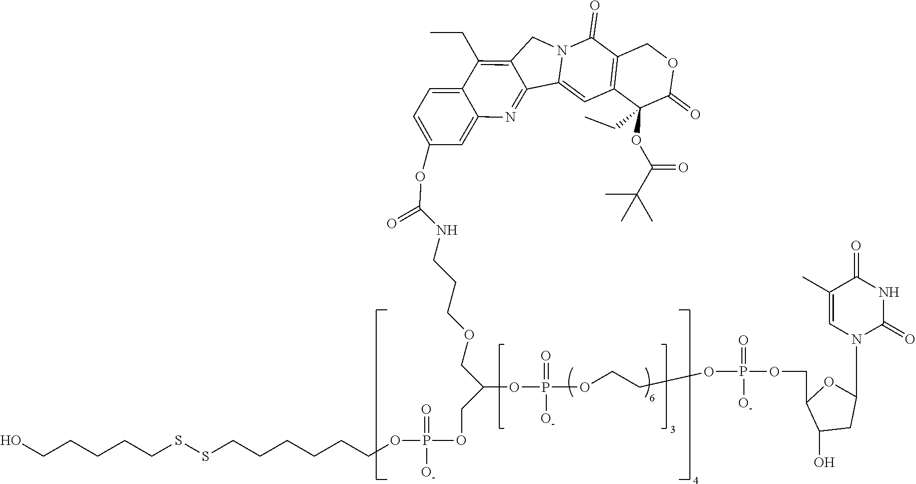 | |
| I-29 | 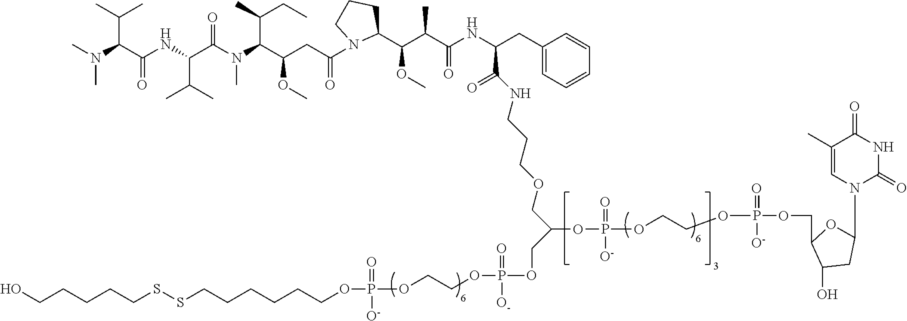 | |
| I-30 | 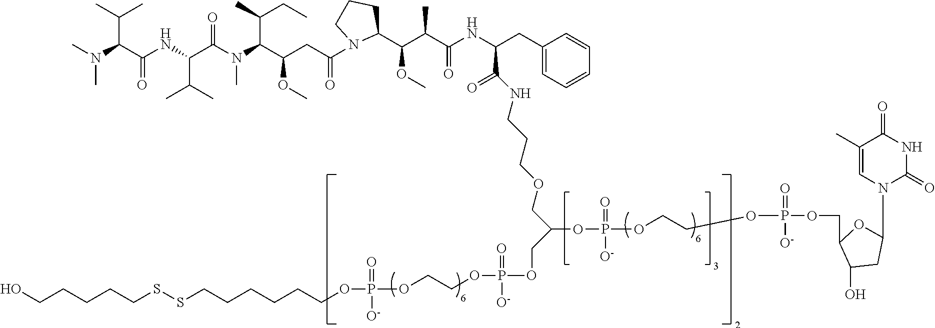 | |
| I-31 | 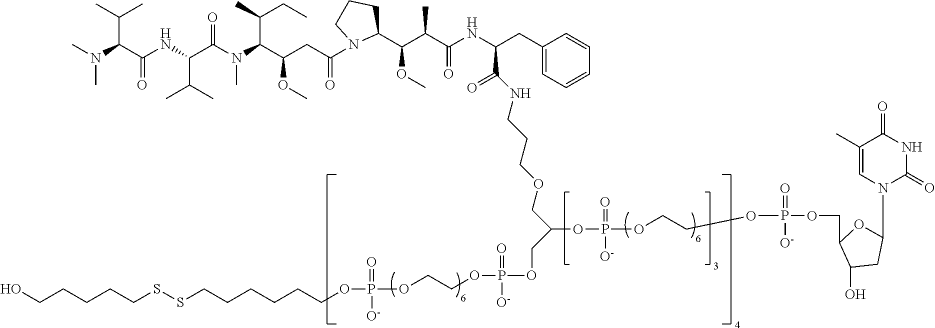 | |
| I-32 | 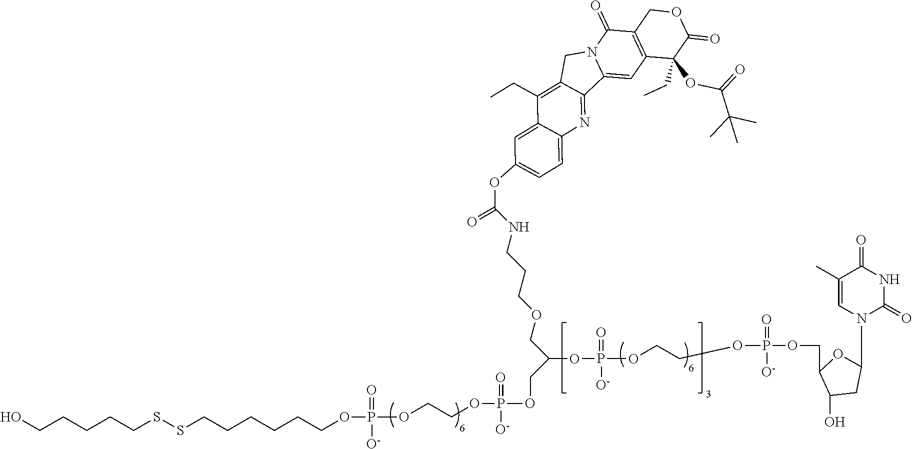 | |
| I-33 | 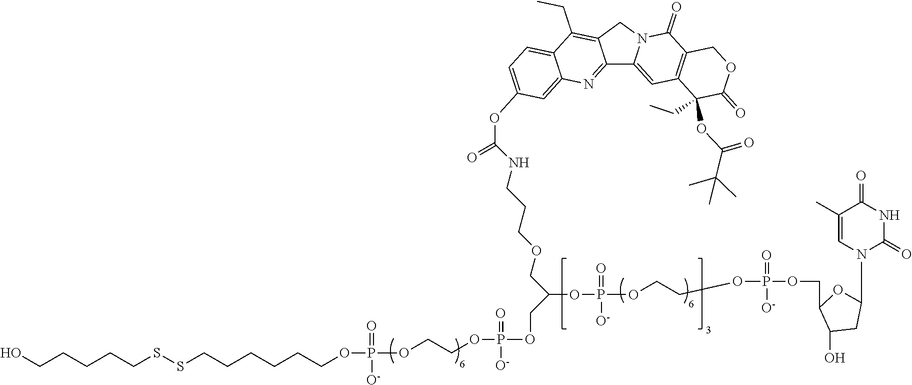 | |
| I-34 | 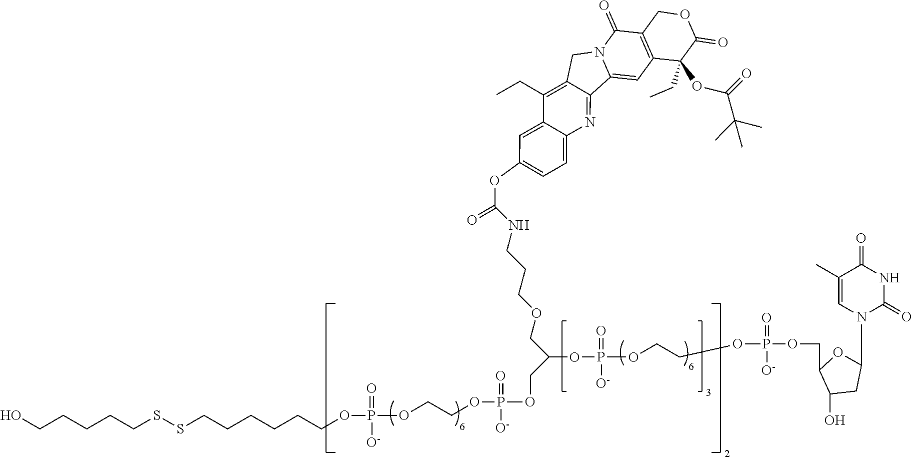 | |
| I-35 | 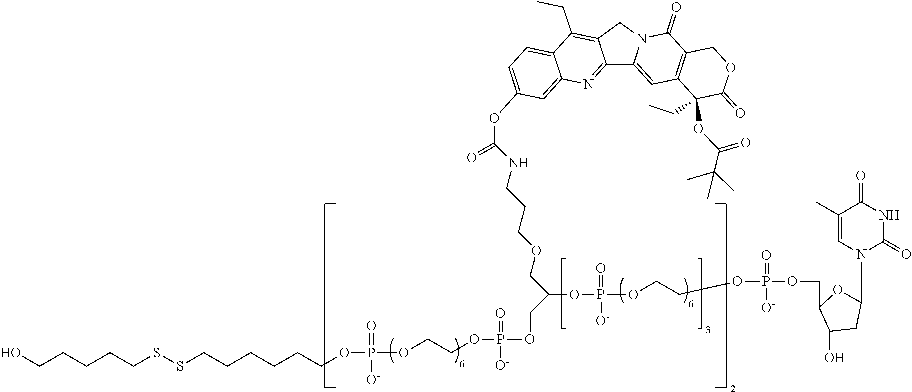 | |
| I-36 | 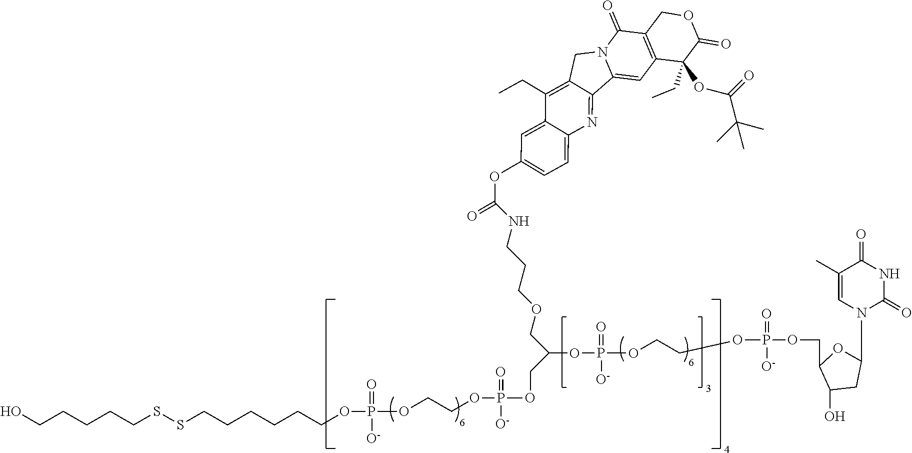 | |
| I-37 | 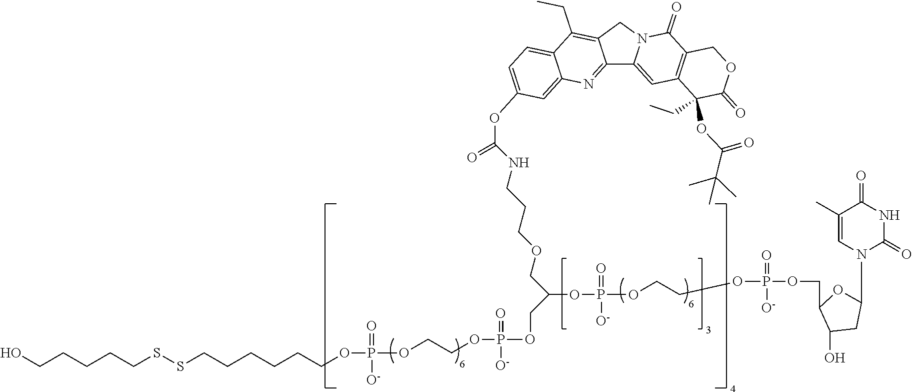 | |
| I-38 | 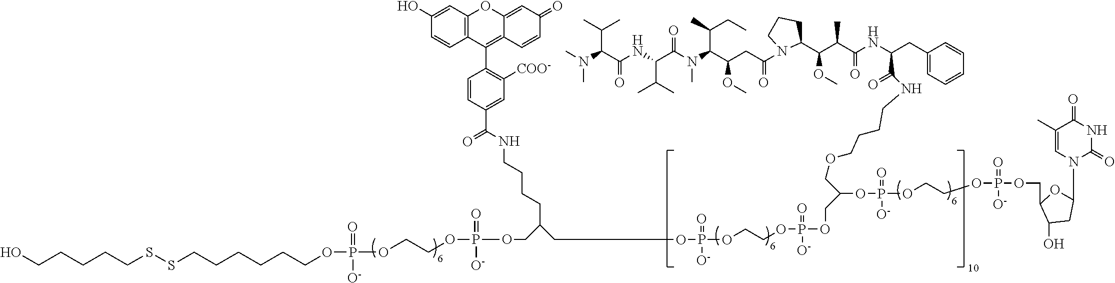 | |
| I-39 | 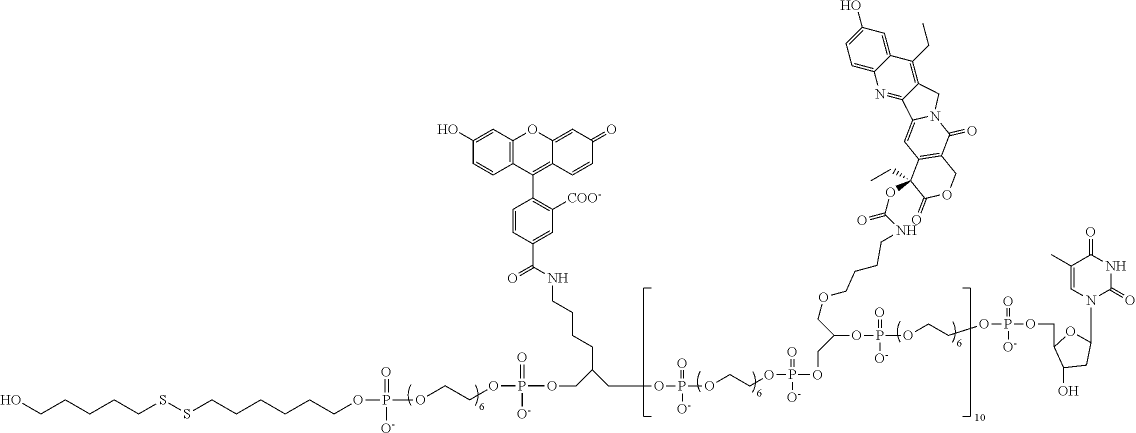 | |
| I-40 | 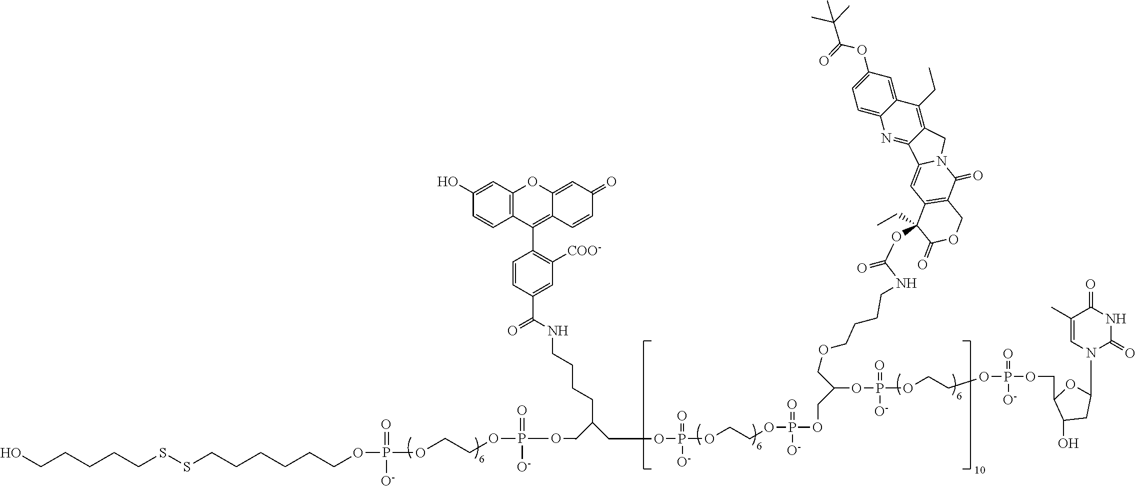 | |
| I-41 | 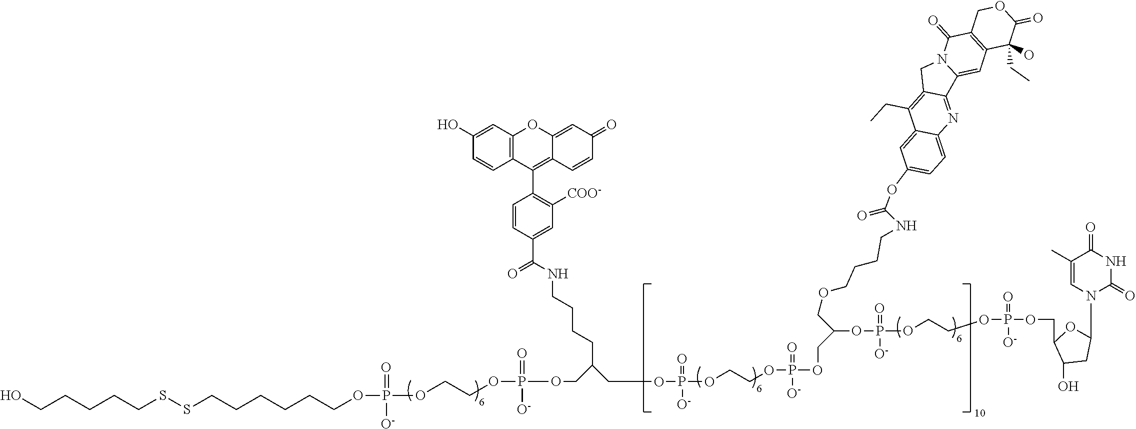 | |
| I-42 | 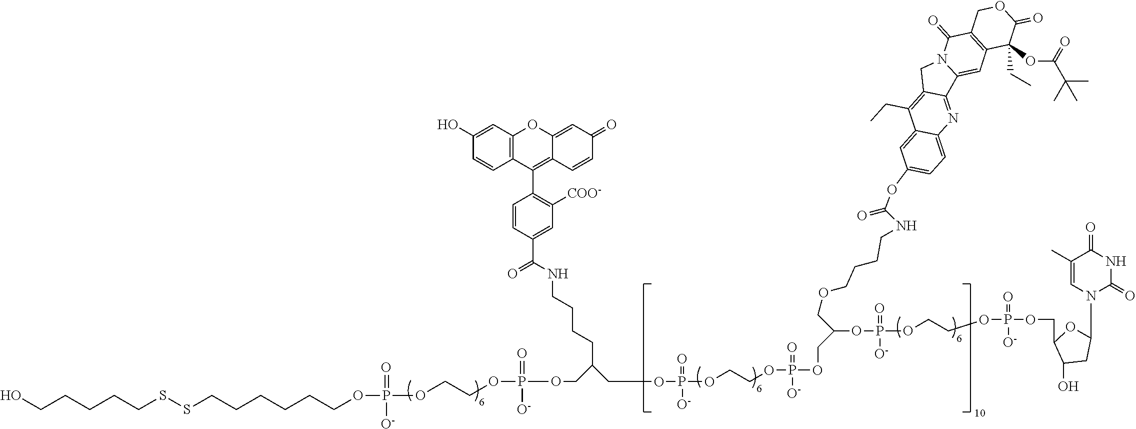 | |
| I-43 | 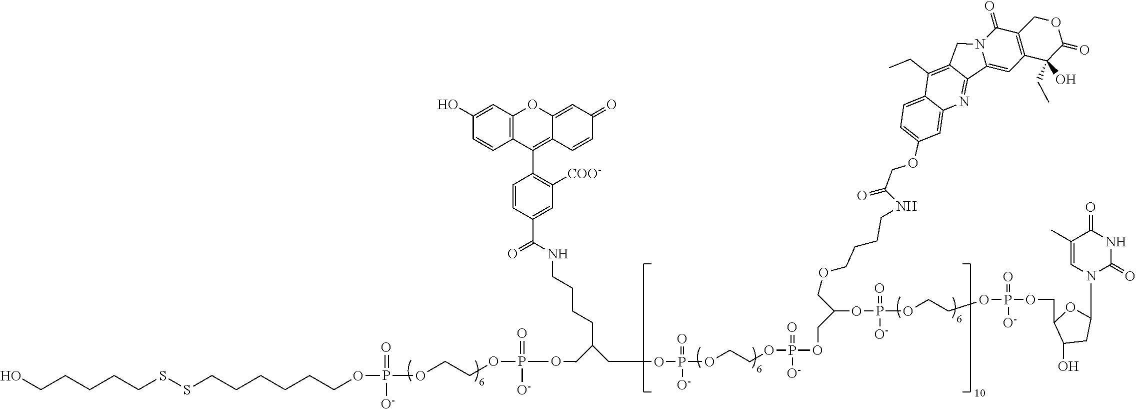 | |
| I-44 | 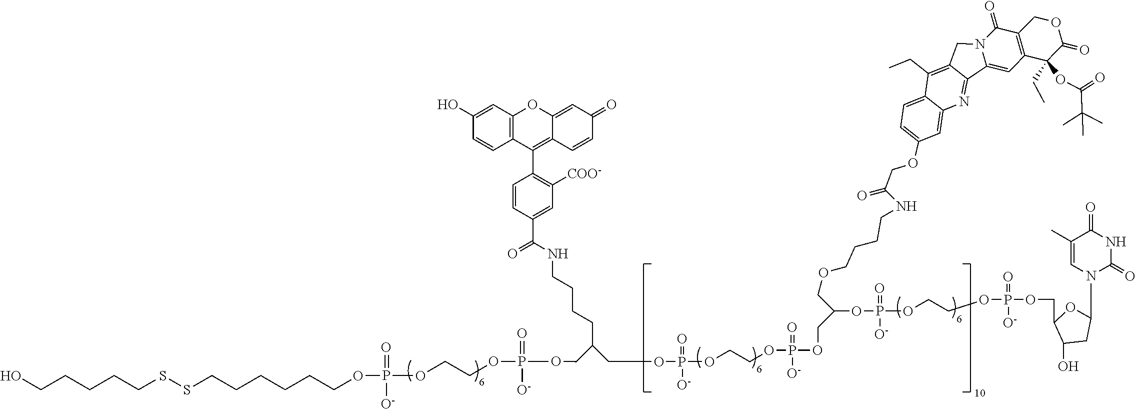 | |

In some embodiments, compounds of the present disclosure (e.g., compounds of Structure (I)) may include a fluorescent or colored moiety attached to the polymer backbone. Any fluorescent and/or colored moiety may be used, for examples those known in the art and typically employed in colorimetric, UV, and/or fluorescent assays may be used. Examples of the fluorescent or colored moieties which are useful in various embodiments of the invention include, but are not limited to: Xanthene derivatives (e.g., fluorescein, rhodamine, Oregon green, eosin or Texas red); Cyanine derivatives (e.g., cyanine, indocarbocyanine, oxacarbocyanine, thiacarbocyanine or merocyanine); Squaraine derivatives and ring-substituted squaraines, including Seta, SeTau, and Square dyes; Naphthalene derivatives (e.g., dansyl and prodan derivatives); Coumarin derivatives; oxadiazole derivatives (e.g., pyridyloxazole, nitrobenzoxadiazole or benzoxadiazole); Anthracene derivatives (e.g., anthraquinones, including DRAQ5, DRAQ7 and CyTRAK Orange); Pyrene derivatives such as cascade blue; Oxazine derivatives (e.g., Nile red, Nile blue, cresyl violet, oxazine 170); Acridine derivatives (e.g., proflavin, acridine orange, acridine yellow); Arylmethine derivatives: auramine, crystal violet, malachite green; and Tetrapyrrole derivatives (e.g., porphin, phthalocyanine or bilirubin). Other exemplary M moieties include: Cyanine dyes, xanthate dyes (e.g., Hex, Vic, Nedd, Joe or Tet); Yakima yellow; Redmond red; tamra; texas red and Alexa fluor® dyes. In some more specific embodiments, compounds of the present disclosure have 6-carboxyfluorescein (6-FAM) or 5-carboxyfluorescein (5-FAM) as a fluorescent moiety attached to the polymer backbone such as shown in compounds I-38-I-44 on Table 1. Compounds containing a fluorescent moiety with repeating units of anticancer therapeutic agents such as Auristatin F (labeled as “AF”) are synthesized according to the procedure described in the present disclosure. Polymers with higher degree of polymerization such as ten (10) repeating units of AF shown in compound I-38 are obtained with high purity. For example, the crude purity (before purifications) of I-38 was 92% by measuring ultra performance liquid chromatography (UPLC).

Compounds of the present disclosure (e.g., compounds of Structure (I)) are useful partly because they may be attached to a targeting molecule (e.g., an antibody or fragment thereof). Such an attachment may be made by reducing a disulfide bond of a compound of Structure (I) with an appropriate reagent (e.g., TCEP) and coupling the resultant molecule to an appropriate linker reagent (e.g., 1,1′-(ethane-1,2-diyl)bis(1H-pyrrole-2,5-dione) which is commonly known as bis-maleimidoethane or “BMOE”). The resultant product can then be coupled to a targeting molecule (e.g., an antibody or fragment thereof) having a free thiol (—SH) group (e.g., present via reduction of a disulfide bond of the targeting molecule).

Accordingly, in some embodiments, R¹ comprises the following structure:

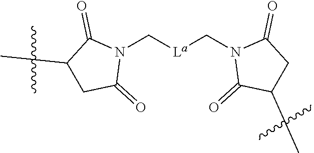

wherein:
L^{a} is a direct bond or C₁-C₆ alkylene. In some embodiments, L^{a} is a direct bond

In some embodiments, R¹ further comprises a covalent bond to an antibody (e.g., monoclonal antibody such as brentuximab, gemtuzumab, trastuzumab, inotuzumab, polatuzumab, enfortumab, trastuzumab, sacituzumab, belantamab, moxetumomab, etc.) or fragment thereof. For example, in some embodiments, R¹ comprises the following structure:

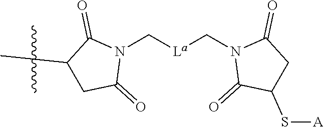

wherein:
A is an antibody (e.g., monoclonal antibody such as brentuximab, gemtuzumab, trastuzumab, inotuzumab, polatuzumab, enfortumab, trastuzumab, sacituzumab, belantamab, or moxetumomab). In some related embodiments, L^{a} is a direct bond. In some embodiments, R¹ has the following structure:

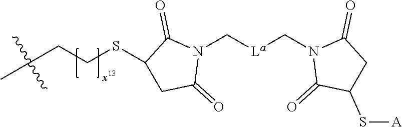

wherein:
x¹³ is 0 or an integer greater than 0 (e.g., 1, 2, 3, 4, 5, 6, 7, 8, 9, 10, 11, or 12).

The present disclosure is also directed at compounds useful as synthetic intermediates for preparing compounds of Structure (I). Accordingly, some embodiments include a compound having the following Structure (II):

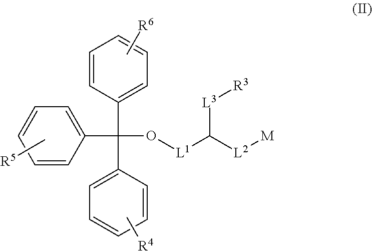

or salt, tautomer, or stereoisomer thereof, wherein:
R³ is H or has the following structure:

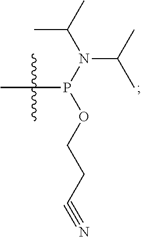 [SMILES: *P(OCCC#N)N(C(C)C)C(C)C]

L¹ a direct bond or an optionally substituted linker;
L² is a linker;
L³ is a direct bond, —O— or —(CH₂)ₘ—O—, wherein m is an integer greater than 0;
R⁴, R⁵, and R⁶ are each independently H or alkoxy; and
M is an anticancer therapeutic.

In some embodiments of Structure (II), L¹ is a C₁-C₆ alkylene linker. In more specific embodiments, L¹ is methylene. In certain embodiments, L³ is —O—, C₁-C₆ alkylene-O—linker, or a direct bond. In certain more specific embodiments, L³ is —O—. In some more specific embodiments, L² heteroalkylene. In certain embodiments, L² comprises oxygen. In more specific embodiments, L² comprises the following structure:

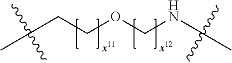

wherein:
x¹¹ and x¹² are each independently an integer greater than 0.

In certain embodiments, x¹¹ is 1, 2, 3, or 4. In certain embodiments, x¹² is 2, 3, 4, or 5. In more specific embodiments, x¹¹ is 1 or 2 and x¹² is 2, 3, or 4. In certain embodiments, L² comprises one of the following structures:

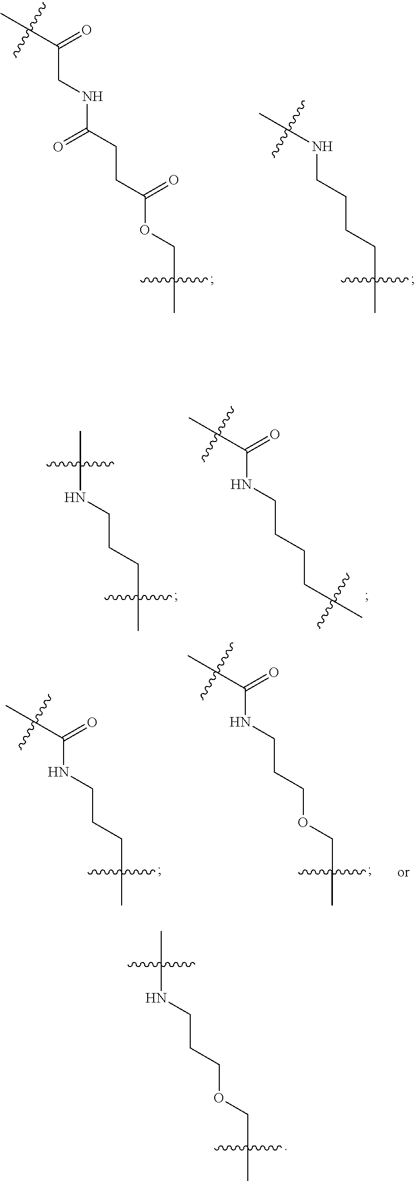

In some embodiments, L² has the following structure:

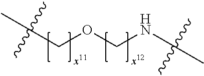

wherein:
x¹¹ and x¹² are each independently an integer greater than 0.

In certain embodiments, x¹¹ is 1, 2, 3, or 4. In some specific embodiments, x¹² is 2, 3, 4, or 5. In some embodiments, x¹¹ is 1 or 2 and x¹² is 2, 3, or 4. In some embodiments, L² further comprises a physiologically cleavable linker. In certain embodiments, L² further comprises an amide bond, an ester bond, a phosphodiester bond, a disulfide bond, a double bond, a triple bond, an ether bond, a hydrazone, an amino acid sequence comprising one or more amino acid residues, a ketone, a diol, a cyano, a nitro, or combinations thereof. In some embodiments, L² comprises an amino acid sequence recognized by a sortase enzyme or cysteine protease. In some embodiments, the amino acid sequence is Leu-Pro-X-Thr-Gly, wherein X is any amino acid residue. In certain embodiments, L² comprises one of the following structures:

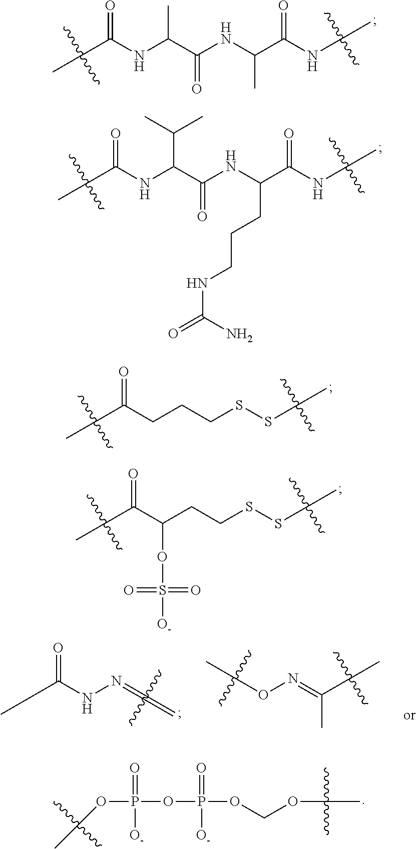

In some embodiments, L² has one of the following structures:

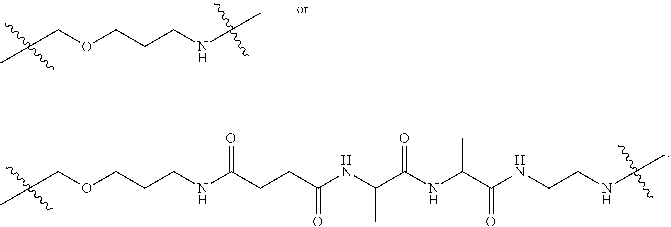 [SMILES: *COCCCN* ; *COCCCNC(=O)CCC(=O)NC(C)C(=O)NC(C)C(=O)NCCN*]

In some embodiments, M is an alkylating agent, an antimetabolite, a microtubule inhibitor, a topoisomerase inhibitor, or a cytotoxic antibiotic. In certain embodiments, M is a nitrogen mustard, a nitrosourea, a tetrazine, an aziridine, a cisplatin or cisplatin derivative, or a non-classical alkylating agent. In some specific embodiments, M is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine. In certain specific embodiments, M is an anti-folate, a fluoropyrimidines, a deoxynucleoside analogue, or a thiopurine. In some more specific embodiments, M is methotrexate, pemetrexed, fluorouracil, capecitabine, cytarabine, gemcitabine, decitabine, azacitidine, fludarabine, nelarabine, cladribine, clofarabine, pentostatin, thioguanine, and mercaptopurine. In certain more specific embodiments, M is an auristatin, a Vinca alkaloid, or a taxane. In some embodiments, M is auristatin F, auristatin E, vincristine, vinblastine, vinorelbine, vindesine, vinflunine, paclitaxel, docetaxel, etoposide, or teniposide. In certain embodiments, M is irinotecan, SN 38, topotecan, camptothecin, doxorubicin, mitoxantrone, teniposide. novobiocin, merbarone, or aclarubicin. In some specific embodiments, M is an anthracycline or a bleomycin. In certain specific embodiments, M is doxorubicin, daunorubicin, epirubicin, idarubicin, pirarubicin, aclarubicin, or mitoxantrone. In some embodiments, M is auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

In some more specific embodiments of Structure (II), the compound has the following Structure (IIA):

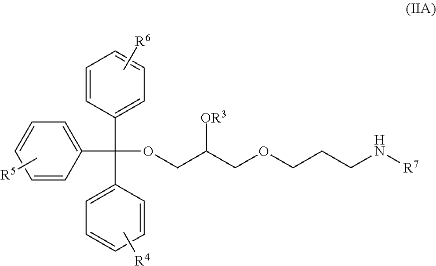

or salt, tautomer, or stereoisomer thereof, wherein:
R³ is H or has the following structure:

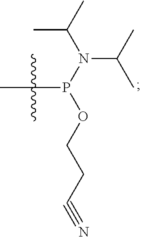 [SMILES: *P(OCCC#N)N(C(C)C)C(C)C]

R⁴, R⁵, and R⁶ are each independently H or alkoxy;
R⁷ has one of the following structures:

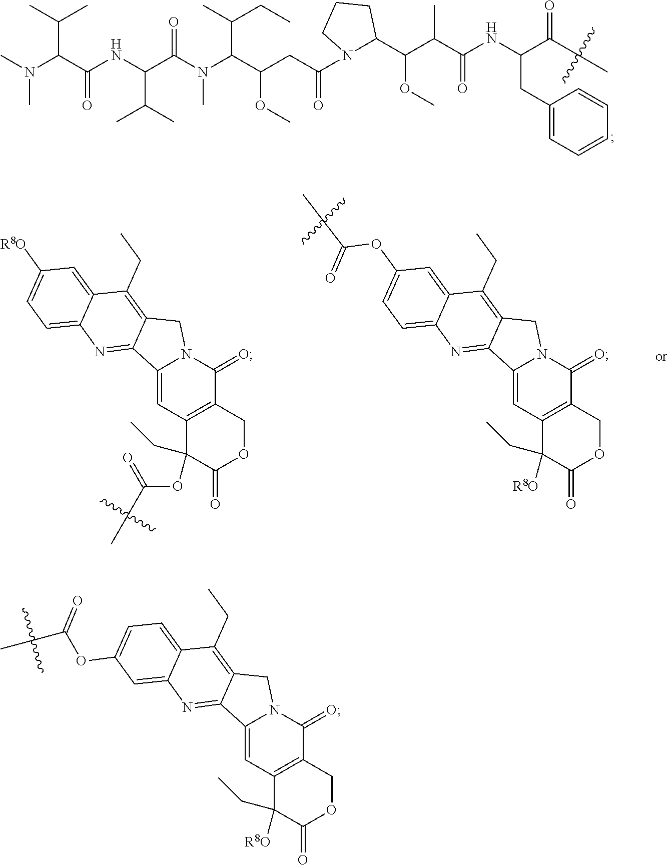

wherein:
R⁸ is H or —C(═O)C(CH₃)₃.

In more specific embodiments, of Structure (II), the compound has the following Structure (IIB):

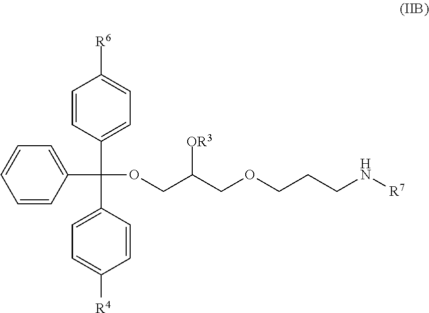

or salt, tautomer, or stereoisomer thereof.

In some more specific embodiments, R³ is H. In certain embodiments, R³ has the following structure:

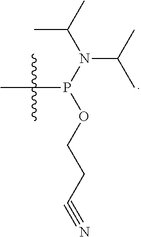 [SMILES: *P(OCCC#N)N(C(C)C)C(C)C]

In some embodiments, R⁴ is alkoxy. In certain embodiments, R⁶ is alkoxy. In some specific embodiments, R⁴ is methoxy. In more specific embodiments, R⁶ is methoxy.

In some embodiments, R⁷ has the following structure:

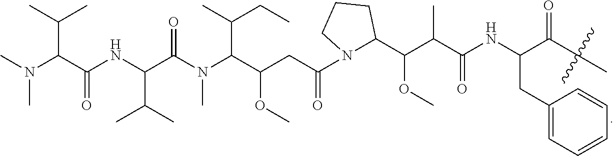 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

In certain embodiments, R⁷ has the following structure:

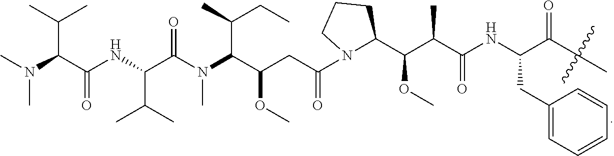

In some embodiments, R⁷ has one of the following structures:

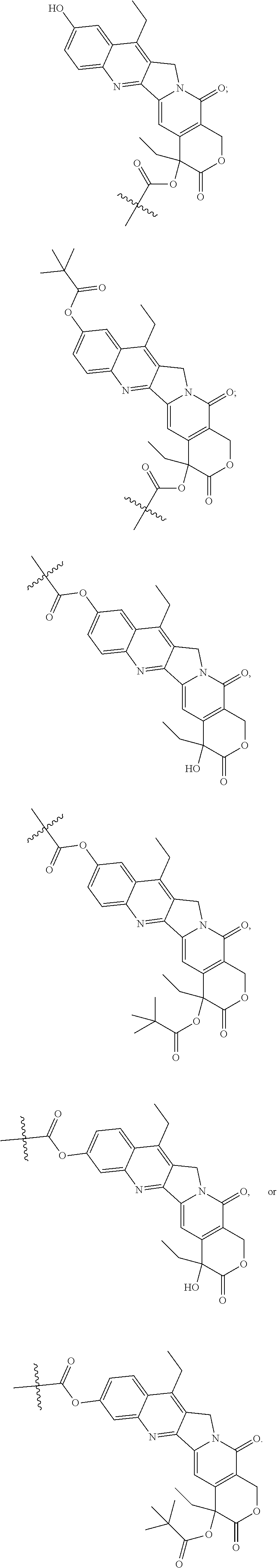 [SMILES: *C(=O)OC1(CC)C(=O)OCc2c1cc1n(c2=O)Cc2c-1nc1ccc(O)cc1c2CC ; *C(=O)OC1(CC)C(=O)OCc2c1cc1n(c2=O)Cc2c-1nc1ccc(OC(=O)C(C)(C)C)cc1c2CC ; *C(=O)Oc1ccc2nc3c(c(CC)c2c1)Cn1c-3cc2c(c1=O)COC(=O)C2(O)CC ; *C(=O)Oc1ccc2nc3c(c(CC)c2c1)Cn1c-3cc2c(c1=O)COC(=O)C2(CC)OC(=O)C(C)(C)C ; *C(=O)Oc1ccc2c(CC)c3c(nc2c1)-c1cc2c(c(=O)n1C3)COC(=O)C2(O)CC ; *C(=O)Oc1ccc2c(CC)c3c(nc2c1)-c1cc2c(c(=O)n1C3)COC(=O)C2(CC)OC(=O)C(C)(C)C]

In certain specific embodiments, R⁷ has one of the following structures:

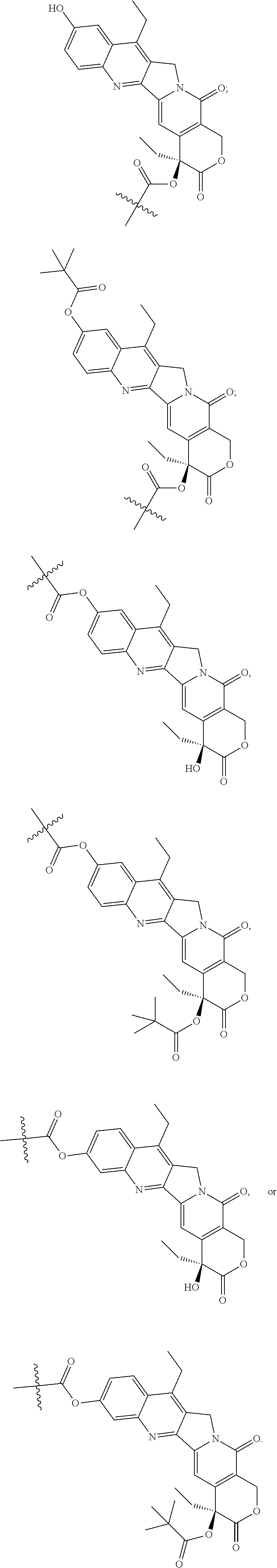 [SMILES: *C(=O)O[C@]1(CC)C(=O)OCc2c1cc1n(c2=O)Cc2c-1nc1ccc(O)cc1c2CC ; *C(=O)O[C@]1(CC)C(=O)OCc2c1cc1n(c2=O)Cc2c-1nc1ccc(OC(=O)C(C)(C)C)cc1c2CC ; *C(=O)Oc1ccc2nc3c(c(CC)c2c1)Cn1c-3cc2c(c1=O)COC(=O)[C@]2(O)CC ; *C(=O)Oc1ccc2nc3c(c(CC)c2c1)Cn1c-3cc2c(c1=O)COC(=O)[C@@]2(CC)OC(=O)C(C)(C)C ; *C(=O)Oc1ccc2c(CC)c3c(nc2c1)-c1cc2c(c(=O)n1C3)COC(=O)[C@]2(O)CC ; *C(=O)Oc1ccc2c(CC)c3c(nc2c1)-c1cc2c(c(=O)n1C3)COC(=O)[C@@]2(CC)OC(=O)C(C)(C)C]

Certain embodiments provide a compound having one of the structures in Table 2 or a salt, stereoisomer, or tautomer thereof

TABLE 2
Representative compounds of Structure (II)

| Com- | | |
|---|---|---|
| pound | | |
| No. | Structure | MW |
| II-1 | 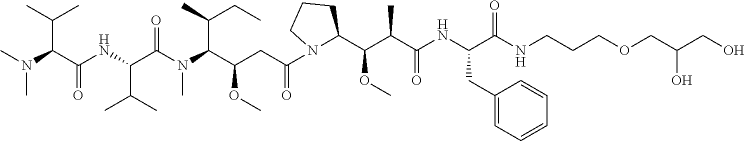 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)CO)OC)N(C)C(=O)[C@@H](NC(=O)[C@H](C(C)C)N(C)C)C(C)C] | 863.15 |
| II-2 | 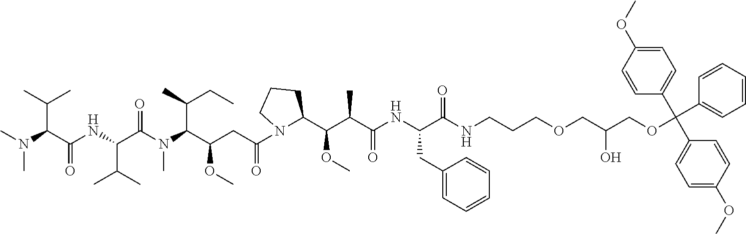 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)COC(c1ccccc1)(c1ccc(OC)cc1)c1ccc(OC)cc1)OC)N(C)C(=O)[C@@H](NC(=O)[C@H](C(C)C)N(C)C)C(C)C] | 1179.55 |
| II-3 | 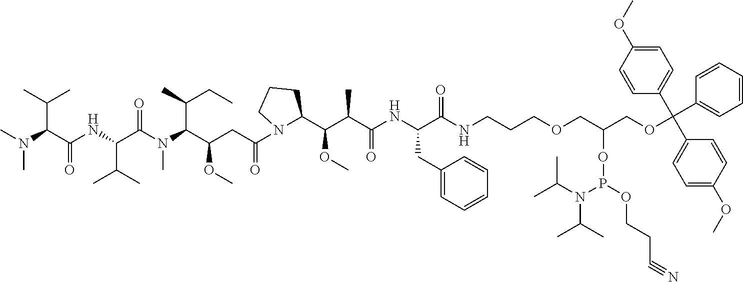 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOCC(COC(c1ccccc1)(c1ccc(OC)cc1)c1ccc(OC)cc1)OP(OCCC#N)N(C(C)C)C(C)C)OC)N(C)C(=O)[C@@H](NC(=O)[C@H](C(C)C)N(C)C)C(C)C] | 1379.77 |
| II-4 | 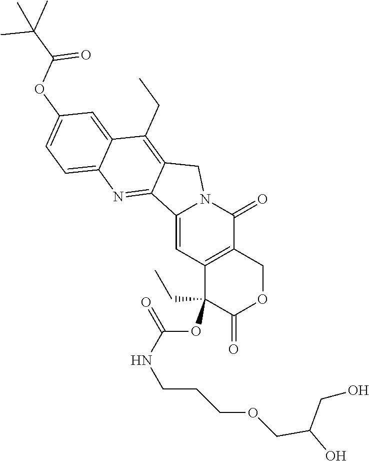 [SMILES: CCc1c2c(nc3ccc(OC(=O)C(C)(C)C)cc13)-c1cc3c(c(=O)n1C2)COC(=O)[C@@]3(CC)OC(=O)NCCCOCC(O)CO] | — |
| II-5 | 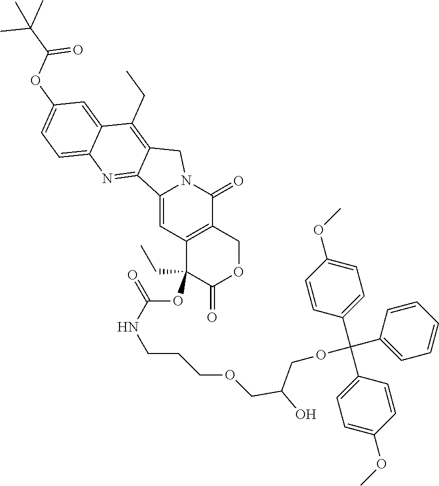 [SMILES: CCc1c2c(nc3ccc(OC(=O)C(C)(C)C)cc13)-c1cc3c(c(=O)n1C2)COC(=O)[C@@]3(CC)OC(=O)NCCCOCC(O)COC(c1ccccc1)(c1ccc(OC)cc1)c1ccc(OC)cc1] | — |
| II-6 | 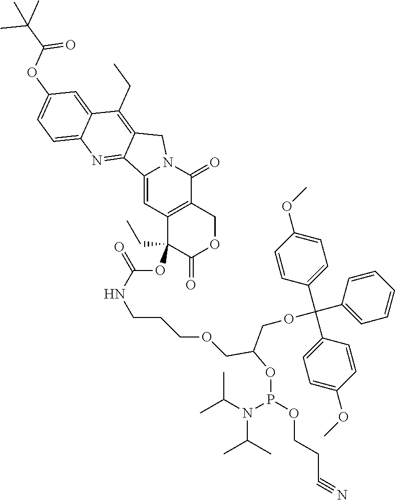 [SMILES: CCc1c2c(nc3ccc(OC(=O)C(C)(C)C)cc13)-c1cc3c(c(=O)n1C2)COC(=O)[C@@]3(CC)OC(=O)NCCCOCC(COC(c1ccccc1)(c1ccc(OC)cc1)c1ccc(OC)cc1)OP(OCCC#N)N(C(C)C)C(C)C] | — |
| II-7 | 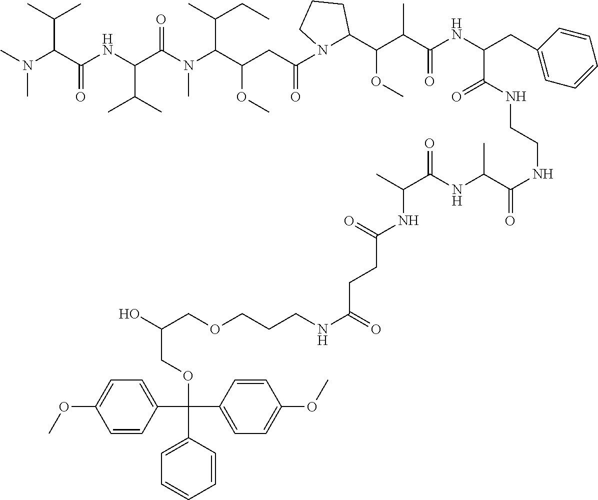 [SMILES: CCC(C)C(C(CC(=O)N1CCCC1C(OC)C(C)C(=O)NC(Cc1ccccc1)C(=O)NCCNC(=O)C(C)NC(=O)C(C)NC(=O)CCC(=O)NCCCOCC(O)COC(c1ccccc1)(c1ccc(OC)cc1)c1ccc(OC)cc1)OC)N(C)C(=O)C(NC(=O)C(C(C)C)N(C)C)C(C)C] | — |
| II-8 | 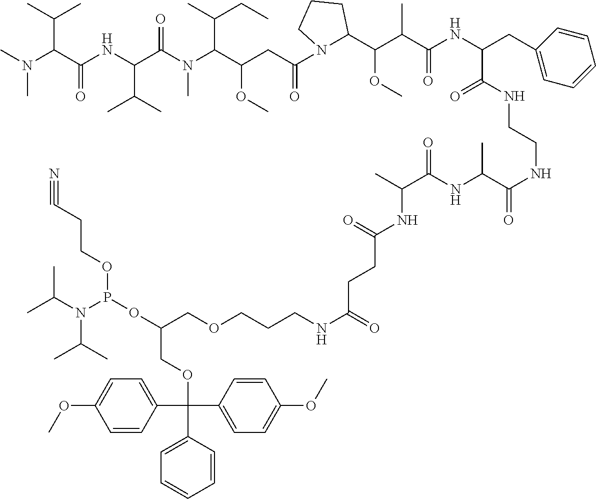 [SMILES: CCC(C)C(C(CC(=O)N1CCCC1C(OC)C(C)C(=O)NC(Cc1ccccc1)C(=O)NCCNC(=O)C(C)NC(=O)C(C)NC(=O)CCC(=O)NCCCOCC(COC(c1ccccc1)(c1ccc(OC)cc1)c1ccc(OC)cc1)OP(OCCC#N)N(C(C)C)C(C)C)OC)N(C)C(=O)C(NC(=O)C(C(C)C)N(C)C)C(C)C] | — |
| II-9 | 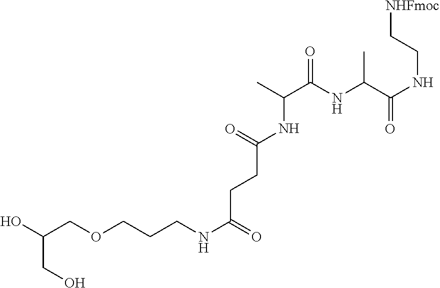 | — |
| II-10 | 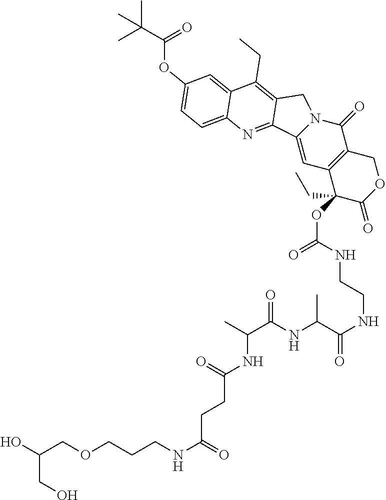 [SMILES: CCc1c2c(nc3ccc(OC(=O)C(C)(C)C)cc13)-c1cc3c(c(=O)n1C2)COC(=O)[C@@]3(CC)OC(=O)NCCNC(=O)C(C)NC(=O)C(C)NC(=O)CCC(=O)NCCCOCC(O)CO] | — |
| I-11 | 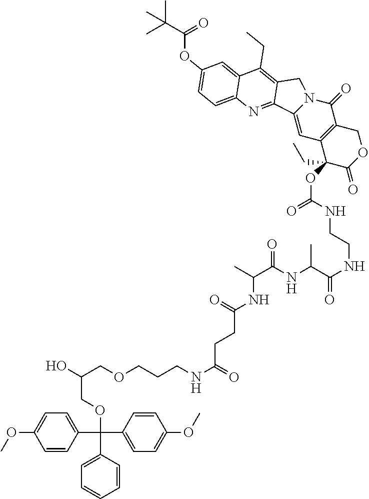 [SMILES: CCc1c2c(nc3ccc(OC(=O)C(C)(C)C)cc13)-c1cc3c(c(=O)n1C2)COC(=O)[C@@]3(CC)OC(=O)NCCNC(=O)C(C)NC(=O)C(C)NC(=O)CCC(=O)NCCCOCC(O)COC(c1ccccc1)(c1ccc(OC)cc1)c1ccc(OC)cc1] | — |
| II-12 | 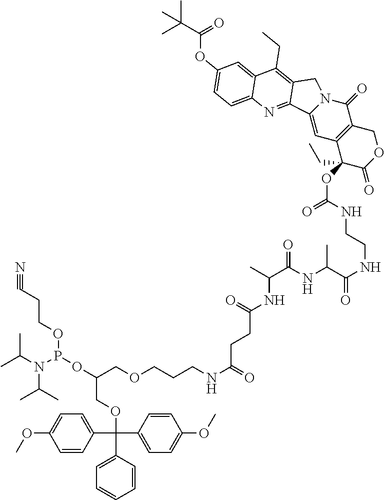 [SMILES: CCc1c2c(nc3ccc(OC(=O)C(C)(C)C)cc13)-c1cc3c(c(=O)n1C2)COC(=O)[C@@]3(CC)OC(=O)NCCNC(=O)C(C)NC(=O)C(C)NC(=O)CCC(=O)NCCCOCC(COC(c1ccccc1)(c1ccc(OC)cc1)c1ccc(OC)cc1)OP(OCCC#N)N(C(C)C)C(C)C] | — |
| II-13 | 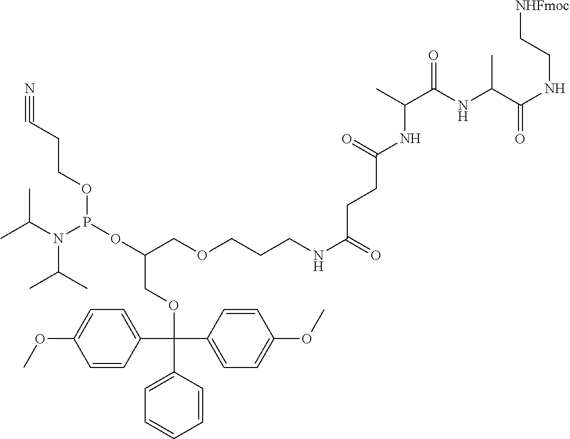 | 2336.1 |
| II-14 | 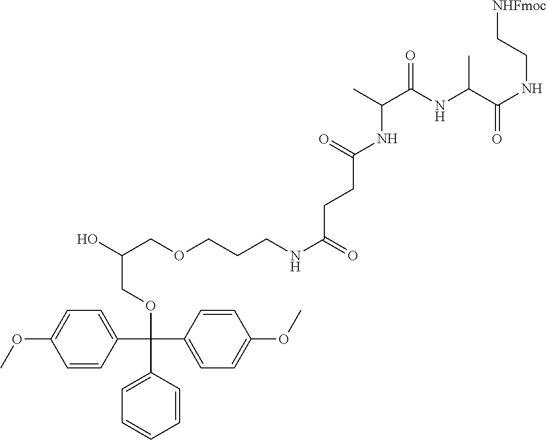 | — |
| II-15 | 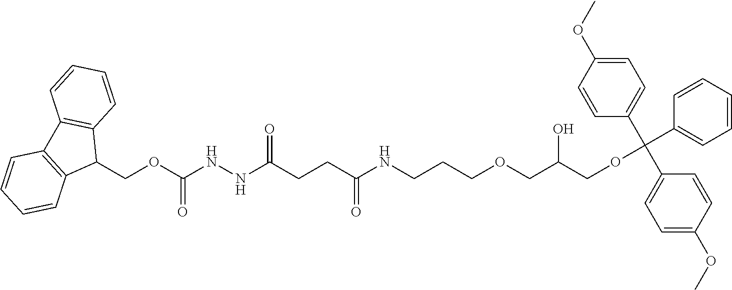 [SMILES: COc1ccc(C(OCC(O)COCCCNC(=O)CCC(=O)NNC(=O)OCC2c3ccccc3-c3ccccc32)(c2ccccc2)c2ccc(OC)cc2)cc1] | |
| II-16 | 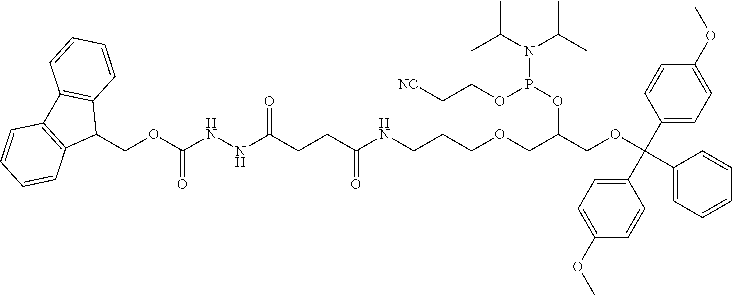 [SMILES: COc1ccc(C(OCC(COCCCNC(=O)CCC(=O)NNC(=O)OCC2c3ccccc3-c3ccccc32)OP(OCCC#N)N(C(C)C)C(C)C)(c2ccccc2)c2ccc(OC)cc2)cc1] | 2167.7 |
| II-17 | 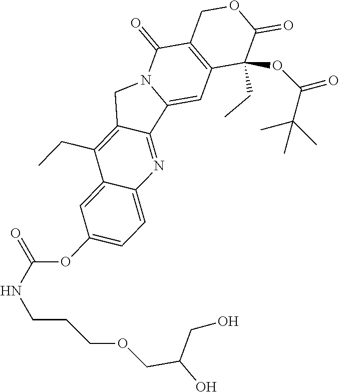 [SMILES: CCc1c2c(nc3ccc(OC(=O)NCCCOCC(O)CO)cc13)-c1cc3c(c(=O)n1C2)COC(=O)[C@@]3(CC)OC(=O)C(C)(C)C] | |
| II-18 | 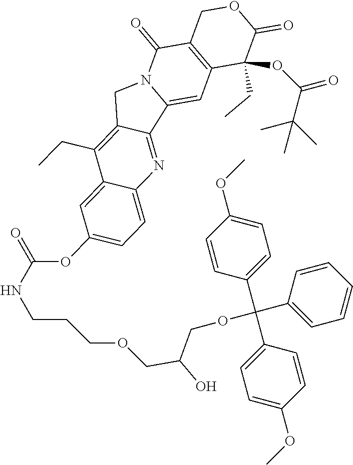 [SMILES: CCc1c2c(nc3ccc(OC(=O)NCCCOCC(O)COC(c4ccccc4)(c4ccc(OC)cc4)c4ccc(OC)cc4)cc13)-c1cc3c(c(=O)n1C2)COC(=O)[C@@]3(CC)OC(=O)C(C)(C)C] | |
| II-19 | 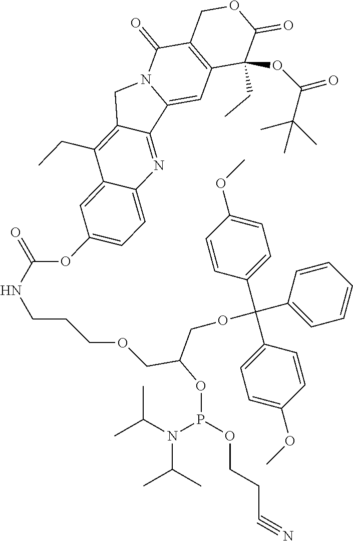 [SMILES: CCc1c2c(nc3ccc(OC(=O)NCCCOCC(COC(c4ccccc4)(c4ccc(OC)cc4)c4ccc(OC)cc4)OP(OCCC#N)N(C(C)C)C(C)C)cc13)-c1cc3c(c(=O)n1C2)COC(=O)[C@@]3(CC)OC(=O)C(C)(C)C] | |
| II-20 | 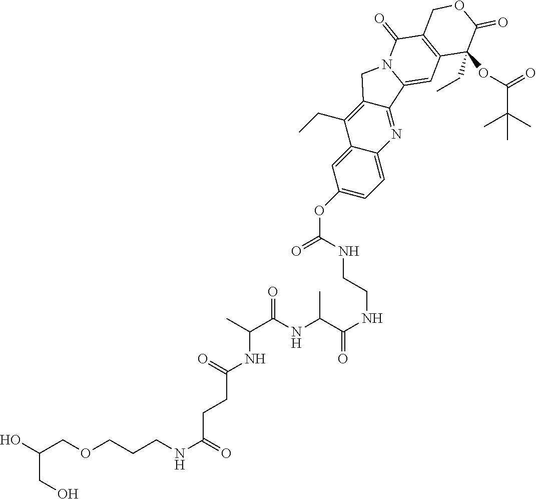 [SMILES: CCc1c2c(nc3ccc(OC(=O)NCCNC(=O)C(C)NC(=O)C(C)NC(=O)CCC(=O)NCCCOCC(O)CO)cc13)-c1cc3c(c(=O)n1C2)COC(=O)[C@@]3(CC)OC(=O)C(C)(C)C] | |
| II-21 | 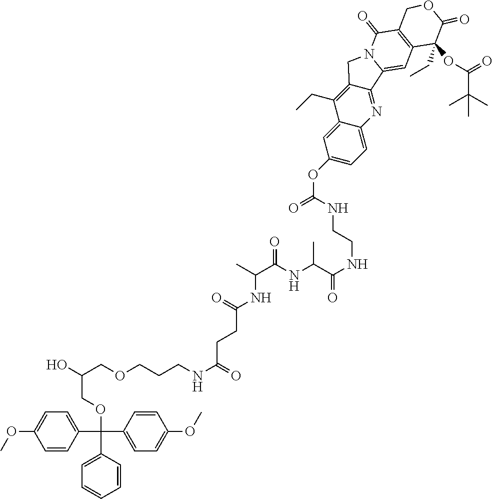 [SMILES: CCc1c2c(nc3ccc(OC(=O)NCCNC(=O)C(C)NC(=O)C(C)NC(=O)CCC(=O)NCCCOCC(O)COC(c4ccccc4)(c4ccc(OC)cc4)c4ccc(OC)cc4)cc13)-c1cc3c(c(=O)n1C2)COC(=O)[C@@]3(CC)OC(=O)C(C)(C)C] | |
| II-22 | 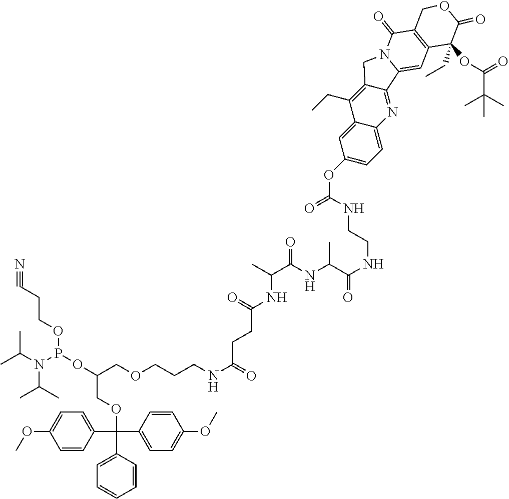 [SMILES: CCc1c2c(nc3ccc(OC(=O)NCCNC(=O)C(C)NC(=O)C(C)NC(=O)CCC(=O)NCCCOCC(COC(c4ccccc4)(c4ccc(OC)cc4)c4ccc(OC)cc4)OP(OCCC#N)N(C(C)C)C(C)C)cc13)-c1cc3c(c(=O)n1C2)COC(=O)[C@@]3(CC)OC(=O)C(C)(C)C] | |
| II-23 | 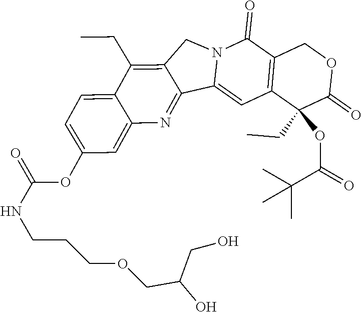 [SMILES: CCc1c2c(nc3cc(OC(=O)NCCCOCC(O)CO)ccc13)-c1cc3c(c(=O)n1C2)COC(=O)[C@@]3(CC)OC(=O)C(C)(C)C] | |
| II-24 | 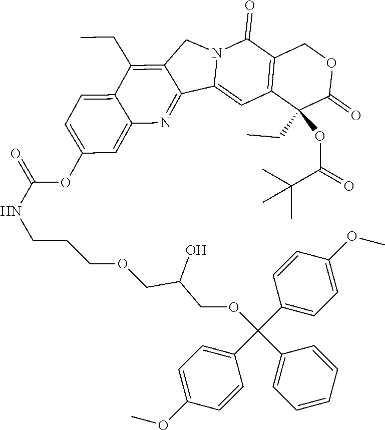 [SMILES: CCc1c2c(nc3cc(OC(=O)NCCCOCC(O)COC(c4ccccc4)(c4ccc(OC)cc4)c4ccc(OC)cc4)ccc13)-c1cc3c(c(=O)n1C2)COC(=O)[C@@]3(CC)OC(=O)C(C)(C)C] | |
| II-25 | 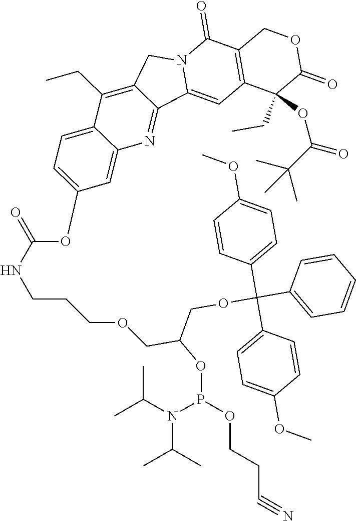 [SMILES: CCc1c2c(nc3cc(OC(=O)NCCCOCC(COC(c4ccccc4)(c4ccc(OC)cc4)c4ccc(OC)cc4)OP(OCCC#N)N(C(C)C)C(C)C)ccc13)-c1cc3c(c(=O)n1C2)COC(=O)[C@@]3(CC)OC(=O)C(C)(C)C] | |
| II-26 | 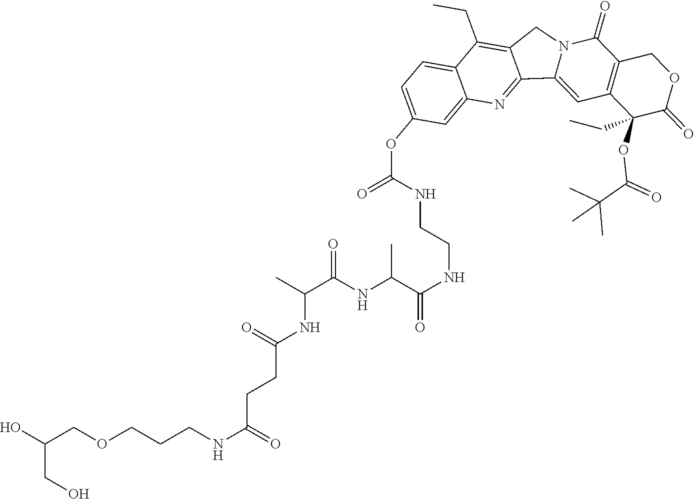 [SMILES: CCc1c2c(nc3cc(OC(=O)NCCNC(=O)C(C)NC(=O)C(C)NC(=O)CCC(=O)NCCCOCC(O)CO)ccc13)-c1cc3c(c(=O)n1C2)COC(=O)[C@@]3(CC)OC(=O)C(C)(C)C] | |
| II-27 | 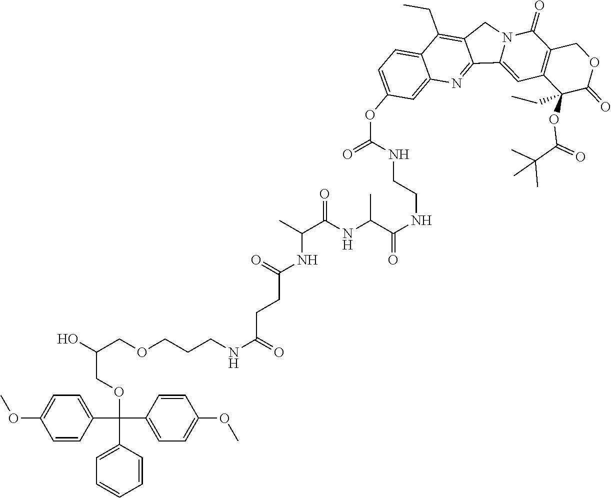 [SMILES: CCc1c2c(nc3cc(OC(=O)NCCNC(=O)C(C)NC(=O)C(C)NC(=O)CCC(=O)NCCCOCC(O)COC(c4ccccc4)(c4ccc(OC)cc4)c4ccc(OC)cc4)ccc13)-c1cc3c(c(=O)n1C2)COC(=O)[C@@]3(CC)OC(=O)C(C)(C)C] | |
| II-28 | 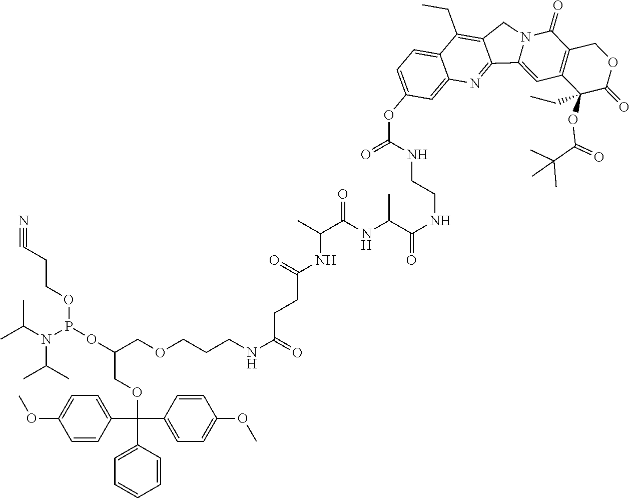 [SMILES: CCc1c2c(nc3cc(OC(=O)NCCNC(=O)C(C)NC(=O)C(C)NC(=O)CCC(=O)NCCCOCC(COC(c4ccccc4)(c4ccc(OC)cc4)c4ccc(OC)cc4)OP(OCCC#N)N(C(C)C)C(C)C)ccc13)-c1cc3c(c(=O)n1C2)COC(=O)[C@@]3(CC)OC(=O)C(C)(C)C] | |

### Pharmaceutical Compositions

One embodiment provides a composition comprising the compound according any one of the embodiments disclosed herein (e.g., a compound of Structure (I)) and a pharmaceutically acceptable carrier.

Other embodiments are directed to pharmaceutical compositions. The pharmaceutical composition comprises any one (or more) of the compounds of Structure (I) and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound of Structure (I) and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound of Structure (I) is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound of Structure (I) is provided in the form of a rapid release formulation, in the form of an extended release formulation, or in the form of an intermediate release formulation. In yet other embodiments, the compound of Structure (I) is administered topically.

The compounds of Structure (I) are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that are used in some embodiments. An exemplary dosage is 10 to 30 mg per day. The exact dosage will depend upon the route of administration, the form in which the compound is administered, the subject to be treated, the body weight of the subject to be treated, and the preference and experience of the attending physician.

In some embodiments, a compound of Structure (I) is administered in a single dose. Typically, such administration will be by injection, e.g., intravenous injection, in order to introduce the agent quickly. However, other routes are used as appropriate. A single dose of a compound of Structure (I) may also be used for treatment of an acute condition.

In some embodiments, a compound of Structure (I) is administered in multiple doses. In some embodiments, dosing is about once, twice, three times, four times, five times, six times, or more than six times per day. In other embodiments, dosing is about once a month, once every two weeks, once a week, or once every other day. In another embodiment a compound of Structure (I) and another agent are administered together about once per day to about 6 times per day. In another embodiment the administration of a compound of Structure (I) and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of Structure (I) may continue as long as necessary. In some embodiments, a compound of Structure (I) is administered for more than 1, 2, 3, 4, 5, 6, 7, 14, or 28 days. In some embodiments, a compound of Structure (I) is administered for less than 28, 14, 7, 6, 5, 4, 3, 2, or 1 day. In some embodiments, a compound of Structure (I) is administered chronically on an ongoing basis, e.g., for the treatment of chronic effects.

In some embodiments, the compounds of Structure (I) are administered in dosages. It is known in the art that due to inter-subject variability in compound pharmacokinetics, individualization of dosing regimen is necessary for optimal therapy. Dosing for a compound of the disclosure may be found by routine experimentation in light of the instant disclosure.

In some embodiments, the compounds of Structure (I) are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound of Structure (I) and a pharmaceutically acceptable diluent(s), excipient(s), or carrier(s). In certain embodiments, the compounds described are administered as pharmaceutical compositions in which compounds of Structure (I) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I).

A pharmaceutical composition, as used herein, refers to a mixture of a compound of Structure (I) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds of Structure (I) provided herein are administered in a pharmaceutical composition to a mammal having a disease, disorder or medical condition to be treated. In specific embodiments, the mammal is a human. In certain embodiments, therapeutically effective amounts vary depending on the severity of the disease, the age and relative health of the subject, the potency of the compound used and other factors. The compounds of Structure (I) are used singly or in combination with one or more therapeutic agents as components of mixtures.

In one embodiment, one or more compounds of Structure (I) is formulated in an aqueous solution. In specific embodiments, the aqueous solution is selected from, by way of example only, a physiologically compatible buffer, such as Hank's solution, Ringer's solution, or physiological saline buffer. In other embodiments, one or more compound of Structure (I) is/are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds (e.g., compounds of Structure (I)) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In still other embodiments, the compounds of Structure (I) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds of Structure (I) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of the compounds of Structure (I) is accomplished by means of iontophoretic patches and the like. In certain embodiments, transdermal patches provide controlled delivery of the compounds of Structure (I). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the compounds of Structure (I) are formulated for administration by inhalation. Various forms suitable for administration by inhalation include, but are not limited to, aerosols, mists or powders. Pharmaceutical compositions of any of compound of Structure (I) are conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator is formulated containing a powder mix of the compound and a suitable powder base such as lactose or starch.

In still other embodiments, the compounds of Structure (I) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with melted cocoa butter.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are optionally used as suitable. Pharmaceutical compositions comprising a compound of Structure (I) are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound of Structure (I), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound of Structure (I) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspensions contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of a compound of Structure (I). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as mermen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

In some embodiments, the concentration of one or more compounds provided in the pharmaceutical compositions is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more compounds is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of one or more compounds is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of one or more compounds ranges from 0.0001 to 10 g, 0.0005 to 9 g, 0.001 to 8 g, 0.005 to 7 g, 0.01 to 6 g, 0.05 to 5 g, 0.1 to 4 g, 0.5 to 4 g, or 1 to 3 g.

### Method of Treatment

Certain compounds of the present disclosure are useful for treating disease (i.e., compounds of Structure (I)). Those compounds disclosed herein offer a targeted approach to drug delivery strategies. Accordingly, one embodiment provides a method of treating a disease (or the symptoms thereof) comprising administering to a mammal (e.g., a human) in need thereof a therapeutically effective amount of a compound of Structure (I).

For example, in certain embodiments the disclosure provides a method of treating solid tumors, multiple myeloma, gliomas, clear cell renal cell carcinoma, prostate cancer, ovarian cancer, non-small cell lung cancer, GI malignancies, acute lymphoblastic leukemia, acute myelogenous leukemia, renal cell carcinoma, colorectal carcinoma, epithelial cancers, pancreatic and gastric cancers, renal cell carcinoma, non-Hodgkin's lymphoma, metastatic renal cell carcinoma, malignant mesothelioma, pancreatic, ovarian, and/or lung adenocarcinoma, B-cell malignancies, breast cancer, melanoma, recurrent multiple myeloma, small cell lung cancer, CD22-positive B cell malignancies, Hodgkin's lymphoma/anaplastic large cell lymphoma, or HER2-positive breast cancer.

In some of the foregoing embodiments, the disease is cancer. For example, in certain embodiments, the cancer is breast cancer, non-Hodgkin's lymphoma, acute myeloid leukemia, multiple myeloma, gastric cancer, renal cell carcinoma, solid tumors, ovarian cancer, prostate cancer, colorectal cancer, pancreatic cancer, small cell lung cancer, diffuse large B-cell lymphoma, a neoplasm, urothelial cancer, ALL, CLL, glioblastoma, Hodgkin's lymphoma, lymphoma, mesothelioma, non-small cell lung cancer, recurrent head and neck cancer, or a combination thereof.

Certain embodiments also relate to a method of treating a hyperproliferative disorder in a mammal (e.g., a human) that comprises administering to said mammal a therapeutically effective amount of a compound of Structure (I), or a pharmaceutically acceptable salt, ester, prodrug, solvate, hydrate or derivative thereof. In some embodiments, said method relates to the treatment of cancer such as acute myeloid leukemia, cancer in adolescents, adrenocortical carcinoma childhood, AIDS-related cancers (e.g., Lymphoma and Kaposi's Sarcoma), anal cancer, appendix cancer, astrocytomas, atypical teratoid, basal cell carcinoma, bile duct cancer, bladder cancer, bone cancer, brain stem glioma, brain tumor, breast cancer, bronchial tumors, Burkitt lymphoma, carcinoid tumor, atypical teratoid, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myleoproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, liver cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, lip and oral cavity cancer, oropharyngeal cancer, ovarian cancer, pancreatic cancer, papillomatosis, paraganglioma, paranasal sinus and nasal cavity cancer, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, stomach (gastric) cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or Viral-Induced cancer. In some embodiments, said method relates to the treatment of a non-cancerous hyperproliferative disorder such as benign hyperplasia of the skin (e.g., psoriasis), restenosis, or prostate (e.g., benign prostatic hypertrophy (BPH)).

Certain particular embodiments provide methods for treatment of lung cancers, the methods comprise administering an effective amount of any of the above described compounds of Structure (I) (or a pharmaceutical composition comprising the same) to a subject in need thereof. In certain embodiments the lung cancer is a non-small cell lung carcinoma (NSCLC), for example adenocarcinoma, squamous-cell lung carcinoma or large-cell lung carcinoma. In other embodiments, the lung cancer is a small cell lung carcinoma. Other lung cancers treatable with the disclosed compounds include, but are not limited to, glandular tumors, carcinoid tumors and undifferentiated carcinomas.

Accordingly, in some embodiments of Structure (I) A is an antibody or a cell surface receptor antagonist. For example, epidermal growth factor receptor (EGFR) inhibitor, a hepatocyte growth factor receptor (HGFR) inhibitor, an insulin-like growth factor receptor (IGFR) inhibitor, a folate, or a MET inhibitor.

In even more embodiments, the method further comprises inducing apoptosis.

In some embodiments, the method of treatment comprises treating a tumor having tumor cells with tumor cell receptors. In some embodiments, the tumor cells have receptors ranging from 1,000 to 100,000, from 1,000 to 50,000, from 1,000 to 25,000 receptors, 1,000 to 10,000 receptors per cell. For example, in some embodiments the tumor cells have about 1,000, about 10,000, or less than 100,000 receptors per cell.

Further therapeutic agents that can be combined with a compound of the disclosure are found in Goodman and Gilman's “The Pharmacological Basis of Therapeutics” Tenth Edition edited by Hardman, Limbird and Gilman or the Physician's Desk Reference, both of which are incorporated herein by reference in their entirety.

The compounds of Structure (I) described herein can be used in combination with the agents disclosed herein or other suitable agents, depending on the condition being treated. Hence, in some embodiments the one or more compounds of the disclosure will be co-administered with other agents as described above. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the disclosure and any of the agents described above can be simultaneously administered, wherein both the agents are present in separate formulations. In another alternative, a compound of the present disclosure can be administered just followed by and any of the agents described above, or vice versa. In some embodiments of the separate administration protocol, a compound of the disclosure and any of the agents described above are administered a few minutes apart, or a few hours apart, or a few days apart.

In some embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In some more specific embodiments, the method further comprises administering an additional therapeutic agent selected from the group consisting of an antineoplastic agent, an enediyne antitumor antibiotic, a maytansinoid, a topoisomerase inhibitor, a kinase inhibitor, an anthracycline, and EGFR inhibitor, an alkylating agent and combinations thereof.

In certain embodiments, the additional therapeutic agent comprises auristatin F, monomethyl auristatin F, monomethyl auristatin E, paciltaxol, SN-38, calicheamicin, anthramycin, abbeymycin, chicamycin, DC-81, mazethramycin, neothramycin A, neothramycin B, porothramycin prothracarcin, sibanomicin, sibiromycin, tomamycin, mertansine, emtansine, irinotecan, camptothecin, topotecan, silatecan, cositecan, Exatecan, Lurtotecan, gimatecan, Belotecan, and Rubitecan.

The examples and preparations provided below further illustrate and exemplify the compounds of the present disclosure and methods of preparing such compounds. It is to be understood that the scope of the present disclosure is not limited in any way by the scope of the following examples and preparations. In the following examples, and throughout the specification and claims, molecules and moieties with a single stereocenter, unless otherwise noted, exist as a racemic mixture. Those molecules and moieties with two or more stereocenters, unless otherwise noted, exist as a racemic mixture of diastereomers. Single enantiomers/diastereomers may be obtained by methods known to those skilled in the art.

### Method of Preparation

The present disclosure is also directed to method of preparing compounds of Structure (I). Accordingly, one embodiment provides a method for preparing a compound of Structure (I):

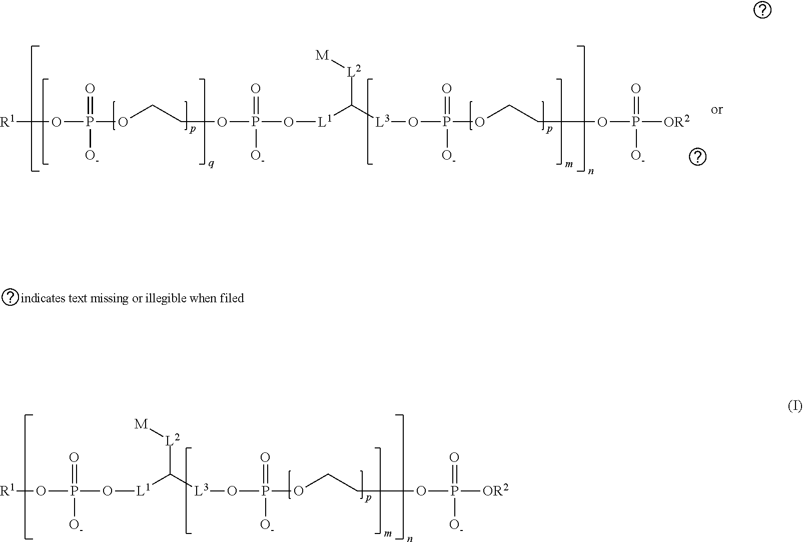

or a pharmaceutically acceptable salt, stereoisomer, or tautomer thereof, wherein
R¹ and R² are independently hydrogen, alkyl, heteroalkyl, Q or a protected form thereof, L′, or have the following structure:

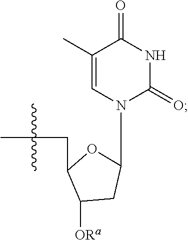

R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0;
p is an integer greater than 0; and
q is an integer greater than 0
   the method comprising:
contacting a first compound having the following structure:

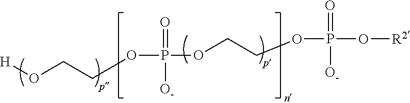

or a salt or tautomer thereof, wherein
R^{2′} comprises a covalent bond to a solid support or solid resin;
n′ is an integer greater than 0;
p′ is an integer greater than 0;
p″ is an integer greater than 0, with a second compound having the following structure:

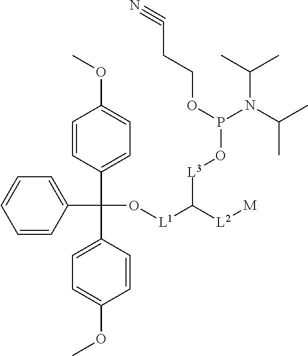

or a salt, tautomer, or stereoisomer thereof,
thereby forming a third compound having the following structure:

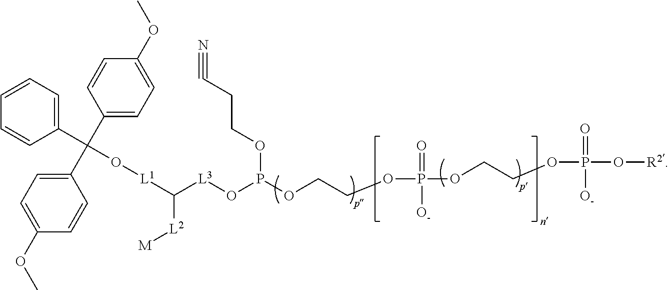

In some embodiments, the method further comprises oxidizing the third compound by contacting the third compound with iodine, water, and a weak base thereby forming a fourth compound having the following structure:

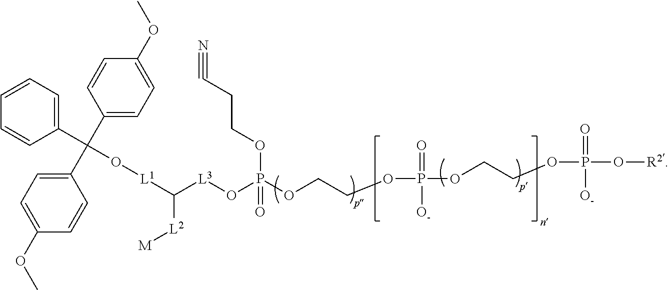

In some embodiments, the weak base is pyridine, lutidine, or collidine.

In certain more specific embodiments, the method further comprises a deprotection step whereby the fourth compound is contacted with a deprotection solution comprising acid, thereby forming a fifth compound having the following structure:

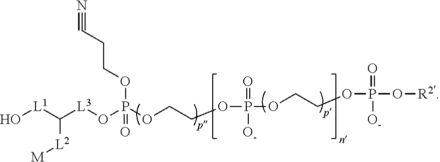

In more specific embodiments, the acid is chloroacetic acid. In certain embodiments, the acid is trichloroacetic acid or dichloroacetic acid. In more specific embodiments, the deprotection solution further comprises dichloromethane or toluene. In some embodiments, the method further comprises removal of 2-cyanoethyl groups. In some more specific embodiments, the removal of 2-cyanoethyl groups comprises treatment with aqueous ammonia. In certain embodiments, the removal of the 2-cyanoethyl groups is prior to the cleavage step. In those specific embodiments, the resultant compound would be as shown above wherein R² comprises a terminal hydrogen.

In some specific embodiments, the solid support or solid resin is controlled pore glass or macroporous polystyrene. In some embodiments, the method is automated. In certain embodiments, the method is fully automated.

In some more specific embodiments, a reaction mixture comprising the third compound is contacted with a capping mixture comprising acetic anhydride and 1-methylimidazole.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin, or a 2-chlorotrityl-chloride resin.

Furthermore, all compounds of the disclosure which exist in free base or acid form can be converted to their salts by treatment with the appropriate inorganic or organic base or acid by methods known to one skilled in the art. Salts of the compounds of the disclosure can be converted to their free base or acid form by standard techniques.

The following Reaction Scheme illustrate exemplary methods of making compounds of this disclosure. It is understood that one skilled in the art may be able to make these compounds by similar methods or by combining other methods known to one skilled in the art. It is also understood that one skilled in the art would be able to make, in a similar manner as described below, other compounds of Structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this disclosure.

DNA synthesis methodology can be applied to build compounds of Structure (I). Monomers (e.g., phosphoramidite monomers) can be purchased commercially (e.g., from ChemGenes Corporation, Wilmington Mass.) or synthesized using methods described herein (see, e.g., Examples 1-3). Introduction of desired moieties can be accomplished during the DNA synthesis steps by including the desired moiety as a portion of the monomer (see, e.g., G¹ of General Reaction Scheme I). An exemplary DNA synthesis scheme is shown below.

### A Representative DNA Synthesis Cycle

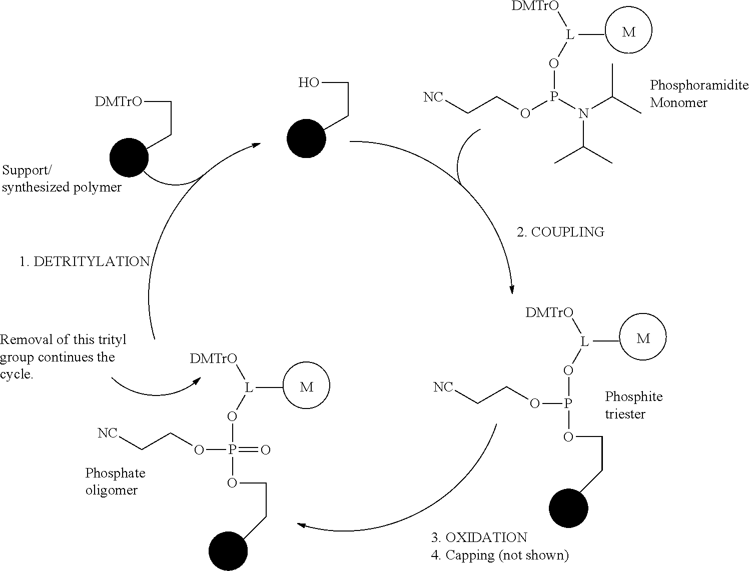

Oligomerization is initiated, typically, through the removal of a protecting group (e.g. a dimethoxytrityl group, DMTr) to reveal a free —OH (hydroxyl) group (Step 1, DETRITYLATION). In a subsequent coupling step, a phosphoramidite monomer is introduced that reacts with the free OH group making a new covalent bond to phosphorus, with concomitant loss of the diisopropyl amine group (Step 2, COUPLING). The resultant, phosphite triester is oxidized (e.g. with I₂ and pyridine) to the more stable phosphate ester (Step 3, OXIDATION) and a capping step renders unreactive any remaining free OH groups (Step 4, CAPPING). The new product, phosphate oligomer, contains a DMTr protected OH group that can be deprotected to reinitiate the synthetic cycle so another phosphoramidite monomer can be appended to the oligomer.

Customization occurs at step 2 through the choice of phosphoramidite monomer. The nature of L (i.e., a linker group) and M (i.e., a chemotherapeutic agent) in the scheme above are selected such that a desired compound of Structure (I) is synthesized. M can be optionally absent to incorporate desired spacing between M moieties. A person of ordinary skill in the art can select multiple monomer types to arrive at compounds of the disclosure containing multiple therapeutic agents and/or other moieties (e.g., fluorophores or chromophores) with concurrent variability in linker groups.

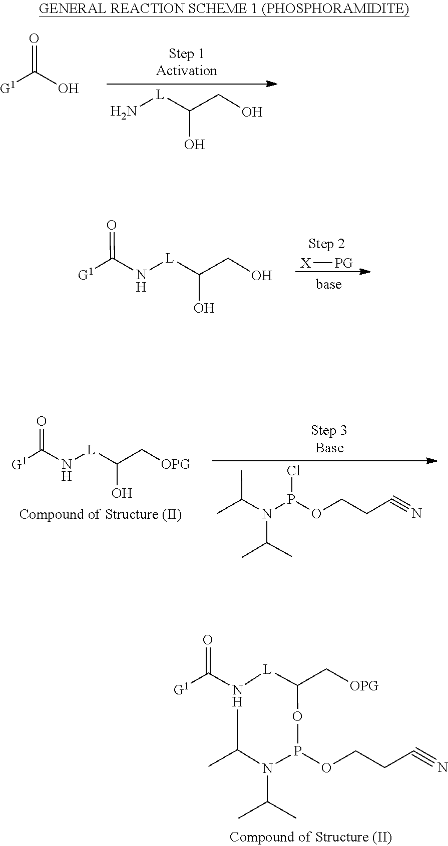

Reaction Scheme I illustrates a method for preparation of phosphoramidite intermediates useful for preparation of compounds of Structure (I). Referring to Reaction Scheme I, G¹ represents a desired moiety containing a carboxylic acid functional group (e.g., a drug moiety such as Auristatin F or SN 38), L represents a bivalent linker moiety (e.g., an alkylene, or alkylene ether), X represents a leaving group (e.g., halo such as Cl), and PG represents a protecting group (e.g., 4,4′-dimethoxytriphenylmethyl). Step 1 of Reaction Scheme I starts with an activation of the carboxylic acid functional group of the first compound shown using known reagents under basic conditions (e.g., HATU and DIPEA in DMF). The activated acid is then reacted with an amine to provide the reaction product of Step 1. The resulting diol is then protected under standard conditions (e.g., 4,4′-dimethoxytriphenylmethyl chloride and pyridine). The protected product is then reacted with 3-((chloro(diisopropylamino)phosphaneyl)oxy)propanenitrile (or other appropriate reagent) to yield a desired compound of Structure (II) as shown above.

The resultant compound of Structure (II) can then be used to synthesize a desired compound of Structure (I) by reaction under well-known (automated) DNA synthesis conditions. In addition to compounds of Structure (II), additional repeat units may be incorporated to achieve a final compound of Structure (I). Generally, compounds having the following structure may be used:

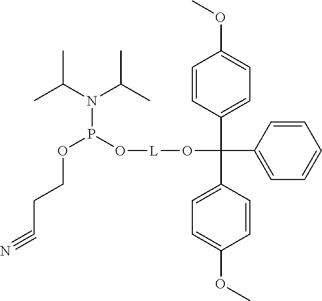

wherein:
L is a desired linker moiety (e.g., including PEG or dye-containing moiety).

In some specific embodiments, the following compound may be used in the synthesis of a compound of Structure (I):

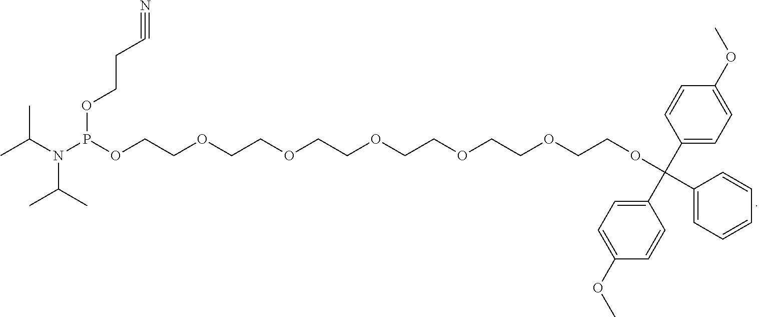 [SMILES: COc1ccc(C(OCCOCCOCCOCCOCCOCCOP(OCCC#N)N(C(C)C)C(C)C)(c2ccccc2)c2ccc(OC)cc2)cc1]

### EXAMPLES

### General Methods

Mass spectral analysis was performed on a Waters/Micromass Quattro micro MS/MS system (in MS only mode) using MassLynx 4.1 acquisition software. Mobile phase used for LC/MS on dyes was 100 mM 1,1,1,3,3,3-hexafluoro-2-propanol (HFIP), 8.6 mM triethylamine (TEA), pH 8. Phosphoramidites and precursor molecules were also analyzed using a Waters Acquity UHPLC system with a 2.1 mm×50 mm Acquity BEH-C18 column held at 45° C., employing an acetonitrile/water mobile phase gradient.

Molecular weights for monomer intermediates were obtained using tropylium cation infusion enhanced ionization on a Waters/Micromass Quattro micro MS/MS system (in MS only mode). Excitation and emission profiles experiments were recorded on a Cary Eclipse spectra photometer.

All reactions were carried out in oven dried glassware under a nitrogen atmosphere unless otherwise stated. Commercially available DNA synthesis reagents were purchased from Glen Research (Sterling, VA). Anhydrous pyridine, toluene, dichloromethane, diisopropylethyl amine, triethylamine, acetic acid, pyridine, and THE were purchased from Aldrich. All other chemicals were purchase from Aldrich or TCI and were used as is with no additional purification.

### Example 1

### Synthesis of Compound 11-1

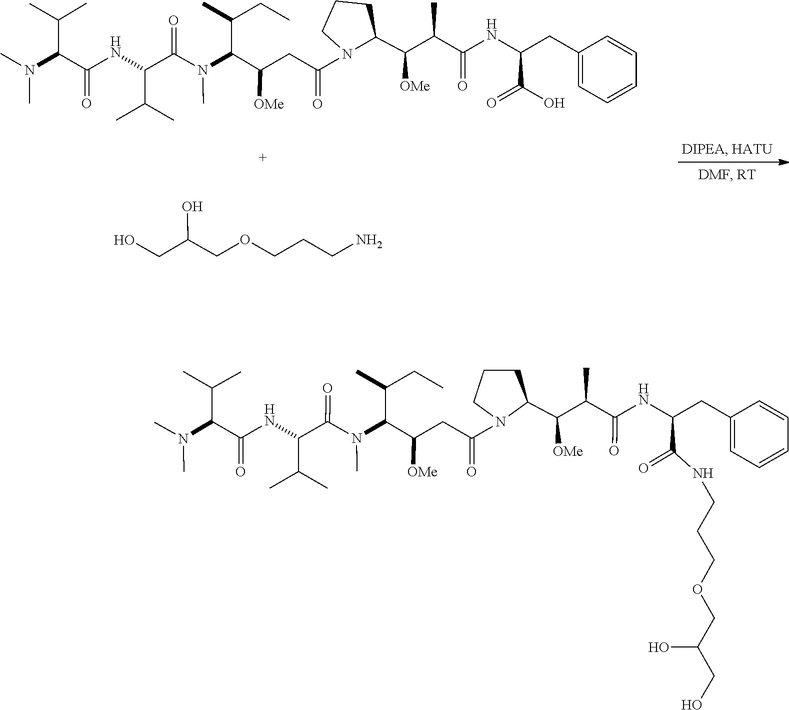 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C)C(C)C ; NCCCOCC(O)CO ; CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)CO)OC)N(C)C(=O)[C@@H](NC(=O)[C@H](C(C)C)N(C)C)C(C)C]

Auristatin F (0.501 g, 0.671 mmole) was added to a dried round bottom flask, under inert gas blanket, with magnetic stir bar, followed by addition of DMF (6.10 mL). The Auristatin F was allowed to dissolve completely under inert gas at room temperature. Then, to the mixture was added DIPEA (0.351 g, 2.013 mmole), followed by addition of HATU (0.278 g, 0.732 mmole). 6,7-Dihydroxy-4-oxaheptylamine (0.091 g, 0.610 mmole) was added to a dried round bottom flask, under inert gas blanket, with magnetic stir bar, followed by addition of DMF (6.10 mL), and dissolve completely at room temperature. Then the Auristatin F reaction mixture was added to the solution containing 6,7-dihydroxy-4-oxaheptylamine; the resultant mixture was allowed to mix at under inert gas, at room temperature, until reaction completion verified by TLC and LC-UV/MS analysis (analytical LC-UV₂₄₈ₙₘ chromatogram showed 12% target product by total peak area or approx. 60% related peak area, identified by MS).

At reaction completion the solvents were stripped off by rotary evaporation, under vacuum (10 mbar), with heating (55° C.). The concentrated residue was placed under full vacuum, at room temperature, for several hours resulting in 1.09 g of crude Compound II-1 by weight (theoretical, 0.535 g, 0.610 mmole).

### Example 2

### Synthesis of Compound 11-2

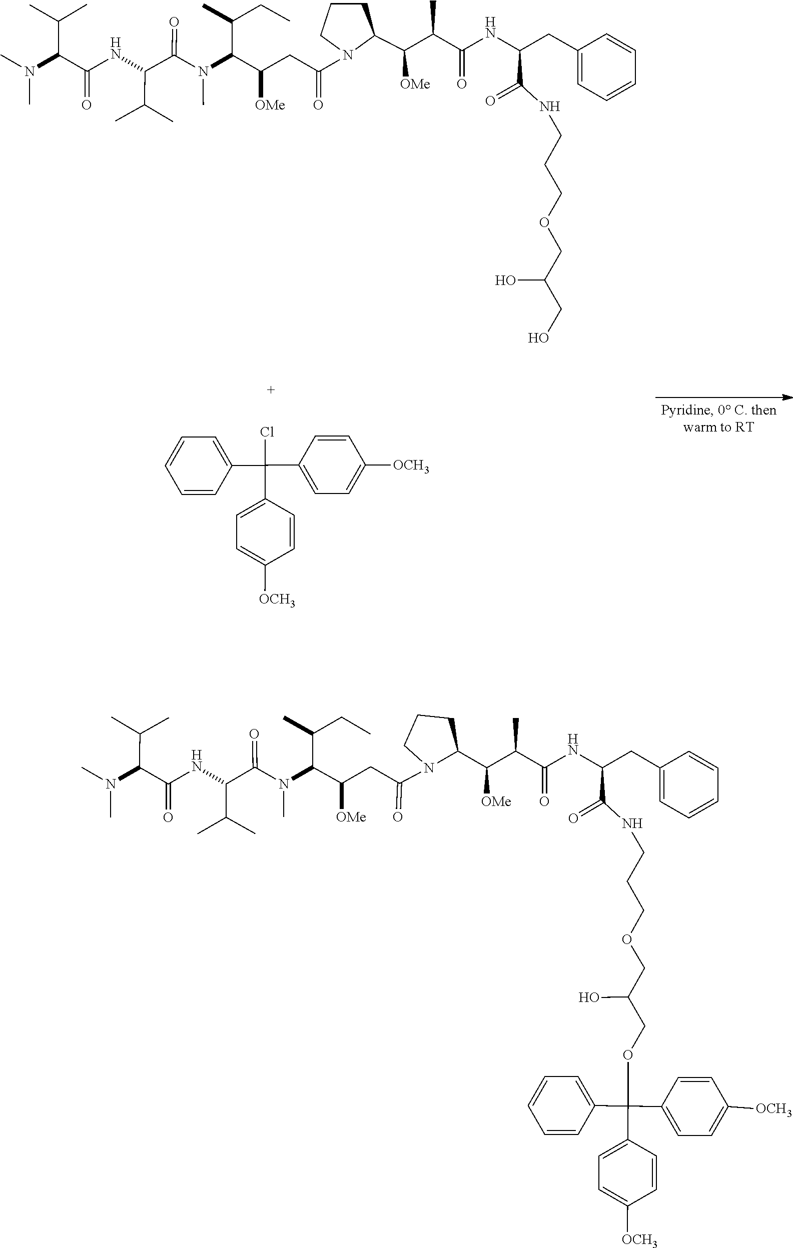 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)CO)OC)N(C)C(=O)[C@@H](NC(=O)[C@H](C(C)C)N(C)C)C(C)C ; COc1ccc(C(Cl)(c2ccccc2)c2ccc(OC)cc2)cc1 ; CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)COC(c1ccccc1)(c1ccc(OC)cc1)c1ccc(OC)cc1)OC)N(C)C(=O)[C@@H](NC(=O)[C@H](C(C)C)N(C)C)C(C)C]

Compound II-1 (a portion of crude material from previous step, 0.444 g, 0.5057 mmole theoretical) was added to a dried round bottom flask, under inert gas blanket, with magnetic stir bar, followed by addition of pyridine, anhydrous (5.06 mL). The reaction flask was then transferred to an ice water bath (0° C.) and allowed to cool with mixing until thermally equalized (approximately 10 minutes). Then, 4,4′-Dimethoxytrityl chloride (0.257 g, 0.759 mmole) was added to the cooled mixture with continuous mixing under inert gas. The reaction mixture was allowed to warm to room temperature then sampled for TLC analysis. When reaction completion verified, the remaining unreacted 4,4′-Dimethoxytrityl chloride was quenched by addition of methanol to the reaction mixture (0.160 g, 5.06 mmole). The solvent was stripped off by rotary evaporation, under vacuum (10 mbar), with heating (55° C.). The concentrated residue was then suspended in toluene (5.06 mL) and toluene stripped off by rotary evaporation, under vacuum (10 mbar), with heating (55° C.); repeated two time. The crude produce was dissolved in dichloromethane (5.06 mL) and washed with sodium bicarbonate (5.06 mL, saturate aq.) and separated, This process was repeated one time. The separated organic phase was washed with sodium chloride (5.06 mL, saturated aq.) and separated. The separated organic phase was dried over sodium sulfate, anhydrous and the sodium sulfate filtered off. The product containing organic phase was sampled for TLC and LC-UV/MS analysis (analytical LC-UV₂₄₈ₙₘ chromatogram showed ˜24% target product by total peak area, identified by MS). Solvent was removed by rotary evaporation, resulting in 0.769 g of crude Compound II-2.

This crude material was then combined with crude material from a small-scale test reaction (0.135 g), for a combined crude yield of 0.931 g. The combined crude material was purified by silica gel flash chromatography, dichloromethane/methanol mobile phase, product containing fractions were pooled and solvent was removed by rotary evaporation, and then placed on vacuum line for at least 24 hours to yield 0.399 g (analytical LC-UV₂₄₈ₙₘ chromatogram showed 82% target product by total peak area, identified by MS).

### Example 3

### Synthesis of Compound II-3

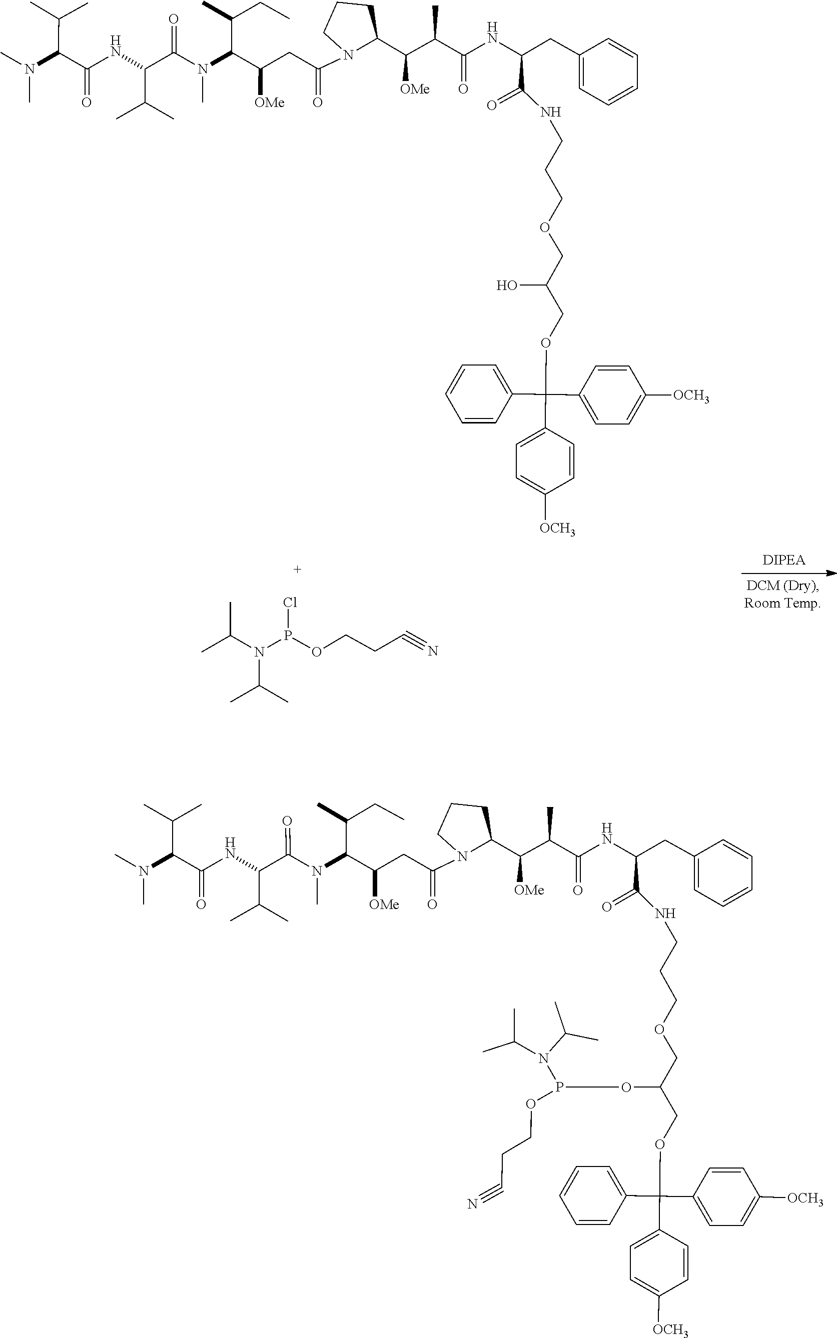 [SMILES: CC[C@H](C)[C@@H]([C@@H](CC(=O)N1CCC[C@H]1[C@H](OC)[C@@H](C)C(=O)N[C@@H](Cc1ccccc1)C(=O)NCCCOCC(O)COC(c1ccccc1)(c1ccc(OC)cc1)c1ccc(OC)cc1)OC)N(C)C(=O)[C@@H](NC(=O)[C@H](C(C)C)N(C)C)C(C)C ; CC(C)N(C(C)C)P(Cl)OCCC#N ; CC[C@H](C)[C@@H]([C@@H](CC(=O)N1CCC[C@H]1[C@H](OC)[C@@H](C)C(=O)N[C@@H](Cc1ccccc1)C(=O)NCCCOCC(COC(c1ccccc1)(c1ccc(OC)cc1)c1ccc(OC)cc1)OP(OCCC#N)N(C(C)C)C(C)C)OC)N(C)C(=O)[C@@H](NC(=O)[C@H](C(C)C)N(C)C)C(C)C]

The purified Compound II-2 (portion of the material 0.267, 0.226 mmole), dried under vacuum for at least 24 hours was dissolved in dichloromethane (2.26 mL), under inert gas blanket, with magnetic stir bar, followed by addition of DIPEA (0.117 g), and then addition of Cl-Phos. (0.107 g). The reaction was allowed to mix for approximately 15 minutes and then sampled for TLC analysis (TLC showed reaction completion). When reaction completion was verified, the reaction mixture was washed by adding directly to sodium bicarbonate (2.26 mL, saturated aq.) and organic phase separated, repeated one time. The organic phases were combined and dried over sodium sulfate, anhydrous, and then the sodium sulfate filtered off. The product containing organic phase was sampled for TLC and LC-UV/MS analysis (analytical LC-UV₂₄₈ₙₘ chromatogram showed two product peaks (diastereomers) ˜64% by total peak area, identified by MS). Then, dichloromethane stripped off by rotary evaporation and proceeded to purification without crude weight. This crude material was then combined with crude material from a small-scale test reaction. The combined crude material was purified by silica gel solid phase extraction, dichloromethane/methanol/triethylamine mobile phase, product containing fractions were pooled (determined by TLC) sampled for TLC and LC-UV/MS analysis. The, mobile phase striped off by rotary evaporation, and then placed on vacuum line for at least 24 hours to yield 0.363 g of Compound II-3. (analytical LC-UV₂₄₈ₙₘ chromatogram showed 68% target product by total peak area, identified by MS).

### Example 4

### Synthesis of Compounds II-13 and II-14

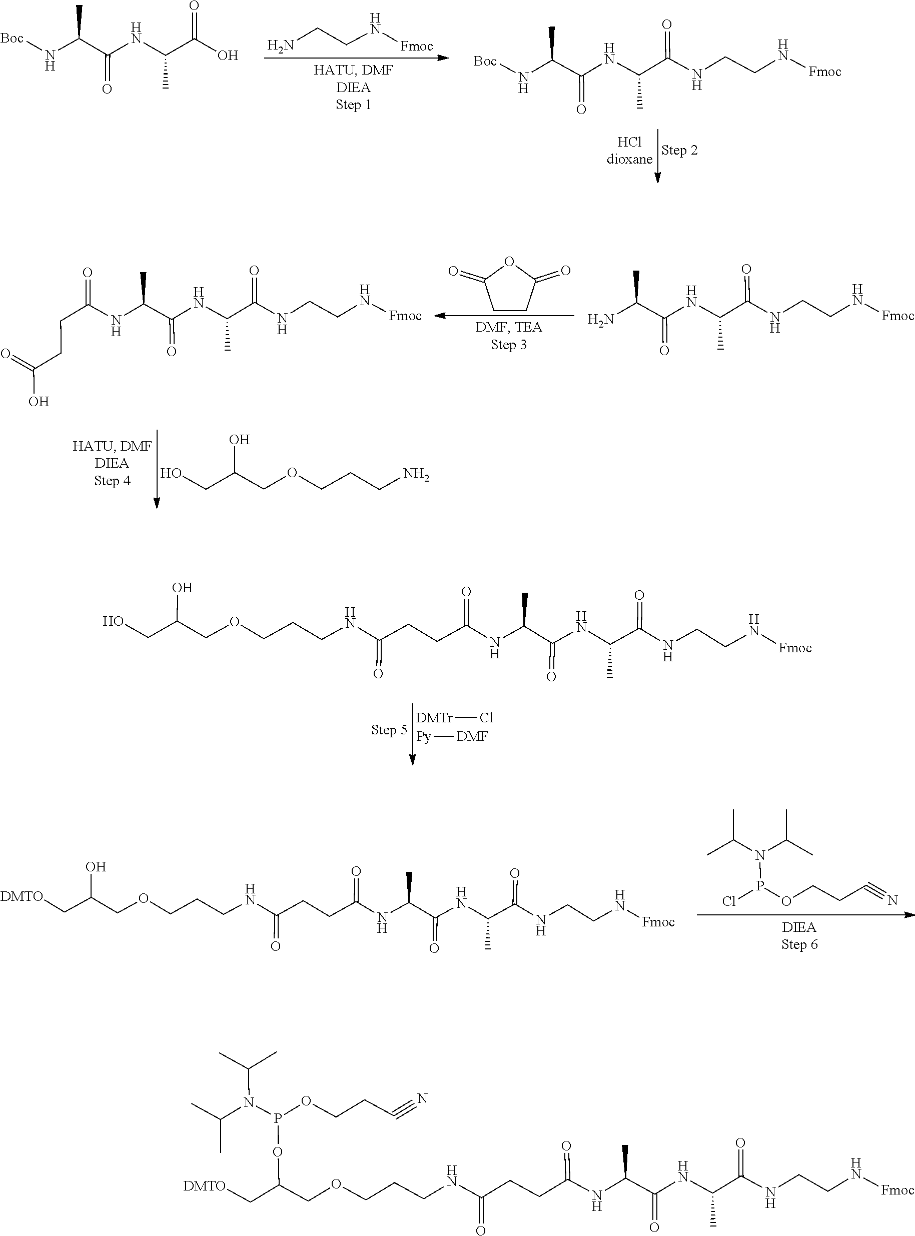

### Step 1

Into a 500 mL round bottomed flask was placed Boc-Ala-Ala-OH (4.0 g, 15.4 mmol, Chem-Impex Cat #04505), DMF (150 mL) and a magnetic stir bar. HATU coupling agent (7.0 g, 18.4 mmol) was added and the mixture was stirred for 5 min before adding Fmoc-1,2-diaminoethane-HCl (4.9 g, 15.4 mmol) and diisopropylethylamine (8.0 mL, 46.1 mmol). After stirring overnight, TLC (silica plats with F254 and dichloromethane:methanol elution 9:1) indicated the reaction was complete. The reaction mixture as concentrated on the rotovap and then partitioned between dichloromethane and water. The solvent layers were separated with an extraction funnel and the aqueous layer extracted three additional times with dichloromethane. The organic layers were combined, dried over sodium sulfate and concentrated under reduced pressure. The product was used in the next step without additional purification.

### Step 2

In a 250 mL round bottomed flask with magnetic stir bar was placed the Boc-Ala-Ala derivative prepared in the previous step (2.5 g, 4.8 mmol). Dichloromethane (30 mL) and DMF (10 mL) were added and the mixture stirred. To this was added 4M hydrochloric acid in dioxane (30 mL, Sigma Cat #345547) and the mixture was stirred. After 1 h, TLC (silica plates with F254, elution with 9:1 dichloromethane:methanol) indicated the reaction was complete. The solvents were removed on the rotovap. Acetonitrile (50 mL) was added and the mixture shaken for a few minutes. The heterogeneous mixture was cooled to 4° C. for 1 h and the solids then collected by filtration (2.0 g). The solids were confirmed to be product by LC-MS.

### Step 3

Into a 250 mL round bottomed flask was placed the amine from the previous step (1.5 g, 3.5 mmol), DMF (35 mL) and a magnetic stir bar. Succinic anhydride (1.8 g, 17.7 mmol) was added in a single portion followed by triethylamine (6.6 mL, 47.2 mmol). The mixture was stirred for 2 h at which point TLC indicated the reaction was complete (TLC elution 4:1 DCM:MeOH). The mixture was concentrated on the rotovap and then treated with potassium carbonate solution (1M, 50 mL). The mixture was allowed to stir for 60 min. The mixture was acidified with hydrochloric acid (20%, 50 mL) and the product precipitated. The mixture was cooled on ice for 30 min and the solids were collected medium frit glass filter. Solid weight 1.32 g. The product was confirmed by LC-MS.

### Step 4

In a small 20 mL glass vial was placed 6,7-dihydroxy-4-oxaheptylamine (443 mg, 3.0 mmol, Berry & Associates Cat #LK4010) and DMF (5 mL). The sample was warmed on a heat plate set a 60° C. for 10 min prior to assembling the other components of the reaction. In a 200 mL round bottomed flask was placed the Succinylated Ala-Ala derivative prepared in the prior step (1.3 g, 2.5 mmol), a magnetic stir bar and DMF (18 mL). HATU (1.1 g, 3.0 mmol, Anaspec) was added and the mixture was stirred for 5 min. To the reaction flask was added with the DMF-amino diol solution in the 20 mL glass vial. The vial was rinsed with 2 mL of DMF and this was added to the reaction vessel. Diisopropylethylamine (1.3 mL, 7.44 mmol) was added, the flask was capped and allowed to stir overnight at room temperature. The magnetic stir bar was removed and the mixture was concentrated on the rotovap and placed on the high vacuum overnight. The reaction was purified on a silica column (Teledyne-Isco) and elution with a dichloromethane:methanol gradient (yield 1.3 g). The product was confirmed by LC-MS.

### Step 5

In a 200 mL round bottomed flask with magnetic stir bar was placed the diol prepared in the prior step (1.3 g, 1.9 mmol) and pyridine (38 mL). 4,4-dimethoxytrityl chloride was added to the solution in a single portion. The flask was capped and allowed to stir overnight. The mixture was concentrated under reduced pressure and the product isolated by flash chromatography 24 g silica column (Teledyne-ISCO) and eluted with a dichloromethane:methanol gradient (yield 540 mg). The product was confirmed by LC-MS.

### Step 6

The tritylated product was placed in 50 mL RB flask with magnetic stir bar (264 mg, 0.28 mmol). Dichloromethane (5.5 mL) and 4 to 5.4 Å molecular sieves were added. 2-Cyanoethyl N,N-diisopropylchlorophosphoramidite (192 μL, 0.6 mmol) and diisopropylethylamine (192 μL, 1.1 mmol) were added via syringe. The reaction was stirred for 2 h. The reaction was monitored by TLC (100% DCM with 5% TEA-prewashed TLC plates). The reaction was concentrated and taken up on silica gel (12 g column—Teledyne-ISCO) and eluted with a hexane:ethylacetate gradient with 2.5% triethylamine. Isolated 122 mg with product confirmation by LC-MS.

### Example 5

### Synthesis of Compound II-15 and II-16

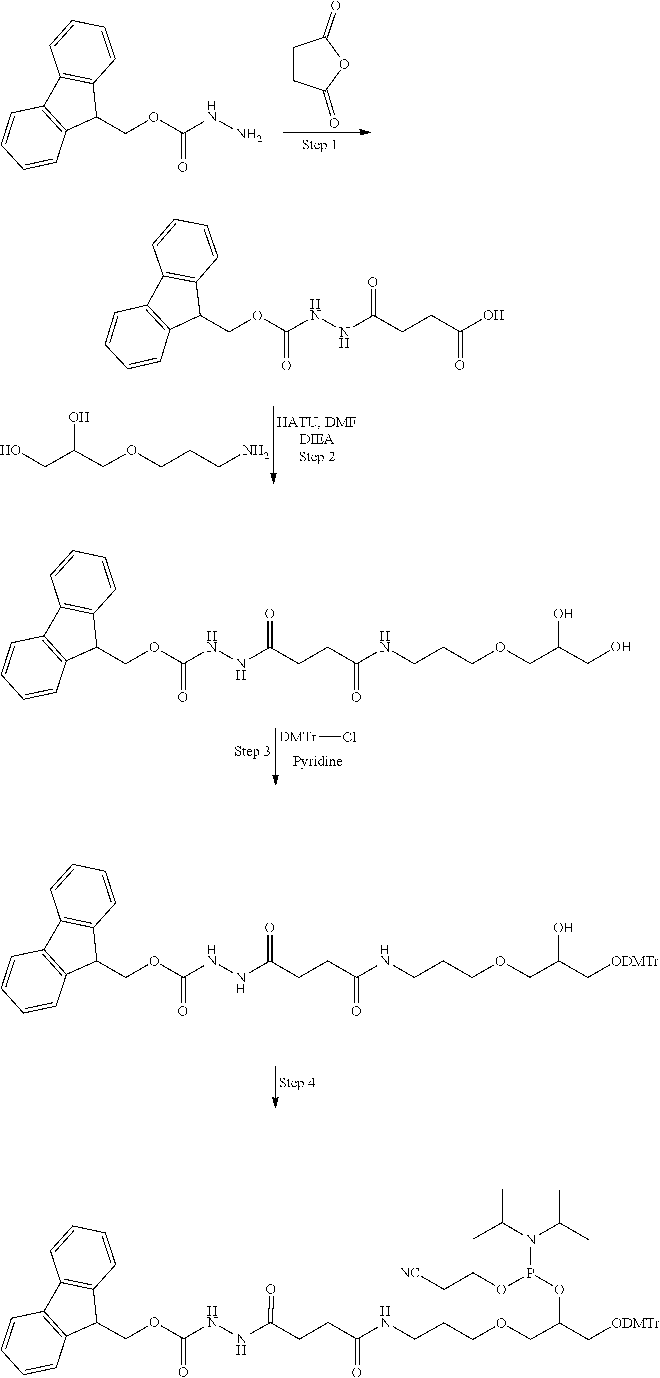

### Step 1

Into a 250 mL round bottomed flask was placed the 9-fluorenylmethyl carbazate (2.0 g, 7.9 mmol, TCI Cat #F0872), DMF (78 mL) and a magnetic stir bar. Succinic anhydride (3.9 g, 39.4 mmol) was added in a single portion followed by triethylamine (6.6 mL, 47.2 mmol). The mixture was stirred for 2 h at which point TLC indicated the reaction was complete (TLC elution 9:1 DCM:MeOH). The mixture was concentrated on the rotovap and then treated with potassium carbonate solution (1M, 50 mL). The mixture was allowed to stir for 60 min during which time gas evolution progressively diminished. The mixture was acidified with hydrochloric acid (20%, 50 mL) and a white solid was formed. The solids were collected by filtration and dried under high vacuum overnight. Solid weight 2.8 g, 100%. The product was confirmed by LC-MS.

### Step 2

In a small 20 mL glass vial was placed 6,7-dihydroxy-4-oxaheptylamine (1.5, 10.2 mmol, Berry & Associates Cat #LK4010) and DMF (8 mL). The sample was warmed on a heat plate set a 60° C. for 10 min prior to assembling the other components of the reaction. In a 250 mL round bottomed flask was placed the carboxy-carbazate solids from the previous step (3.0 g, 8.47 mmol), a magnetic stir bar and DMF (75 mL). The peptide coupling agent HATU (3.9 g, 10.2 mmol, Anaspec) was added and the mixture was stirred for 5 min. To the reaction flask was added with the DMF-amino diol solution in the 4 mL glass vial. The vial was rinsed with 2 mL of DMF and this was added to the reaction vessel. Finally, diisopropylethylamine (4.4 mL, 25.4 mmol) was added in a single portion, the flask was capped and allowed to stir overnight at room temperature. The magnetic stir bar was removed and the reaction was concentrated on the rotovap and then placed on the high vacuum overnight. The reaction was purified on a 40 g silica gel column (Teledyne-Isco) and elution with a dichloromethane:methanol gradient (yield 1.42 g). The product was confirmed by LC-MS.

### Step 3

In a 250 mL round bottomed flask with magnetic stir bar was placed the diol prepared in the previous step (1.4 g, 2.9 mmol) and pyridine (58 mL). To this solution was added 4,4-dimethoxytrityl chloride in a single portion, the flask was capped and allowed to stir overnight. The mixture was concentrated under reduced pressure and the product isolated by flash chromatography on a 40 g silica column (Teledyne-ISCO) and eluted with a dichloromethane:methanol gradient (yield 1.32 g). The product was confirmed by LC-MS.

### Step 4

The purified tritylated product from the previous step was placed in 200 mL round bottomed flask with magnetic stir bar (1.1 g, 1.4 mmol). The flask was charged with dichloromethane (28 mL) and a small scoop of 4 Å molecular sieves. 2-Cyanoethyl N,N-diisopropylchlorophosphoramidite (342 μL, 1.5 mmol) and diisopropylethylamine (850 μL, 4.9 mmol) were added via syringe in a back and forth manner until both reagents were added. The reaction was stirred for 2 h. The reaction was monitored by TLC (100% DCM with 5% TEA-prewashed TLC plates). The reaction was concentrated and taken up on silica gel (24 g column—Teledyne-ISCO) and eluted with a hexane:ethylacetate gradient with 2.5% triethylamine. Isolated 375 mg with product confirmation by LC-MS.

The resultant compound of the above reaction scheme can form an acid labile linker when deprotected (i.e., Fmoc removed) and coupled to an aldehyde or ketone moiety. That is, the deprotected hydrazine can react with an appropriate aldehyde or ketone according to the generalized reaction scheme shown below:

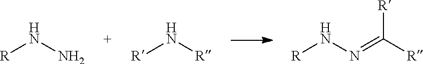

### Example 6

### Synthesis of Compound II-17

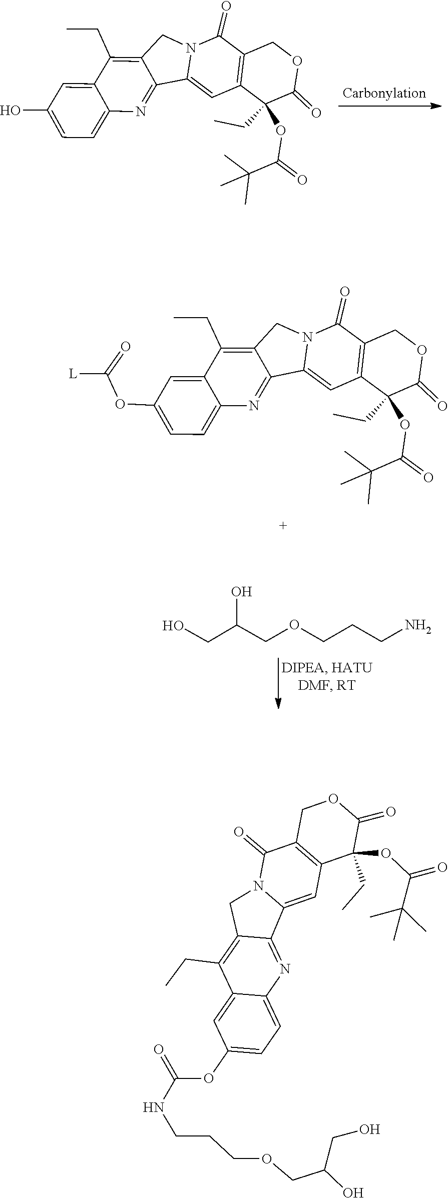

Camptothecin derivative (MW: 476.53 g/mol; 0.320 g, 0.671 mmole) is carbonylated to afford the carbonylated camptothecin derivative with one of the following known carbonylation reactions: 1) phosgene reagent such as phosgene, diphosgene, or triphosgene to afford a carbonylated camptothecin derivative where L is Cl; 2) aryl chloroformate reagents such as phenyl chloroformate or 4-nitrophenyl chloroformate to afford a carbonylated camptothecin derivative where L is phenoxy in case of phenyl chloroformate or 4-nitrophenoxy in case of 4-nitrophenyl chloroformate; 3) haloalkyl chloroformate reagents such as trifluoroethyl chloroformate or chloroethylchloroformate to afford a carbonylated camptothecin derivative where L is trifluoroethoxy in case of trifluoroethyl chloroformate or chloroethoxy in case of chloroethylchloroformate; or 4) carbonyl diheterocyclyl reagents such as carbonyldiimidazole to afford a carbonylated camptothecin derivative where L is 1-imidazolyl.

Carbonylated Camptothecin derivative prepared according to the above carbonylation is added to a dried round bottom flask, under inert gas blanket, with magnetic stir bar, followed by addition of DMF (6.10 mL). The carbonylated Camptothecin derivative is allowed to dissolve completely under inert gas at room temperature. Then, to the mixture is added DIPEA (0.351 g, 2.013 mmole), followed by addition of HATU (0.278 g, 0.732 mmole). 6,7-Dihydroxy-4-oxaheptylamine (0.091 g, 0.610 mmole) is added to a dried round bottom flask, under inert gas blanket, with magnetic stir bar, followed by addition of DMF (6.10 mL), and dissolve completely at room temperature. Then the carbonylated Camptothecin derivative reaction mixture is added to the solution containing 6,7-dihydroxy-4-oxaheptylamine; the resultant mixture is allowed to mix at under inert gas, at room temperature, until reaction completion verified by TLC and LC-UV/MS analysis (analytical LC-UV₂₄₈ₙₘ chromatogram showed 12% target product by total peak area or approx. 60% related peak area, identified by MS).

At reaction completion the solvents are stripped off by rotary evaporation, under vacuum (10 mbar), with heating (55° C.). The concentrated residue is placed under full vacuum, at room temperature, for several hours resulting in crude Compound II-17. Compound II-23 can also be prepared similarly with the camptothecin derivative with a hydroxyl group at 11 position instead of 10 position.

### Example 7

### Synthesis of Compound II-18

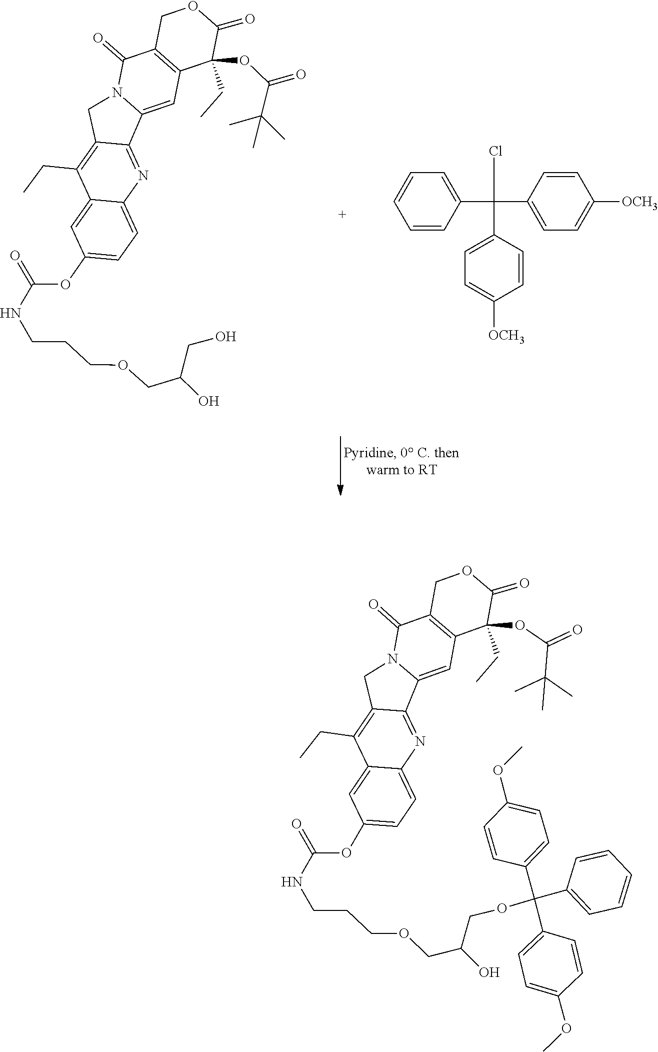 [SMILES: CCc1c2c(nc3ccc(OC(=O)NCCCOCC(O)CO)cc13)-c1cc3c(c(=O)n1C2)COC(=O)[C@@]3(CC)OC(=O)C(C)(C)C ; COc1ccc(C(Cl)(c2ccccc2)c2ccc(OC)cc2)cc1 ; CCc1c2c(nc3ccc(OC(=O)NCCCOCC(O)COC(c4ccccc4)(c4ccc(OC)cc4)c4ccc(OC)cc4)cc13)-c1cc3c(c(=O)n1C2)COC(=O)[C@@]3(CC)OC(=O)C(C)(C)C]

Compound II-17 (a portion of crude material from previous step, 0.330 g, 0.5057 mmole theoretical) is added to a dried round bottom flask, under inert gas blanket, with magnetic stir bar, followed by addition of pyridine, anhydrous (5.06 mL). The reaction flask is then transferred to an ice water bath (0° C.) and allowed to cool with mixing until thermally equalized (approximately 10 minutes). Then, 4,4′-Dimethoxytrityl chloride (0.257 g, 0.759 mmole) is added to the cooled mixture with continuous mixing under inert gas. The reaction mixture is allowed to warm to room temperature then sampled for TLC analysis. When reaction completion verified, the remaining unreacted 4,4′-Dimethoxytrityl chloride is quenched by addition of methanol to the reaction mixture (0.160 g, 5.06 mmole). The solvent is stripped off by rotary evaporation, under vacuum (10 mbar), with heating (55° C.). The concentrated residue is then suspended in toluene (5.06 mL) and toluene stripped off by rotary evaporation, under vacuum (10 mbar), with heating (55° C.); repeated two time. The crude produce is dissolved in dichloromethane (5.06 mL) and washed with sodium bicarbonate (5.06 mL, saturate aq.) and separated. This process is repeated one time. The separated organic phase is washed with sodium chloride (5.06 mL, saturated aq.) and separated. The separated organic phase is dried over sodium sulfate, anhydrous and the sodium sulfate filtered off. The product containing organic phase is sampled for TLC and LC-UV/MS analysis. Solvent is removed by rotary evaporation, resulting in crude Compound II-18.

The combined crude material is purified by silica gel flash chromatography, dichloromethane/methanol mobile phase, product containing fractions are pooled and solvent is removed by rotary evaporation, and then placed on vacuum line for at least 24 hours to yield Compound II-18. Compound II-24 can also be prepared similarly from starting with Compound II-23.

### Example 8

### Synthesis of Compound II-19

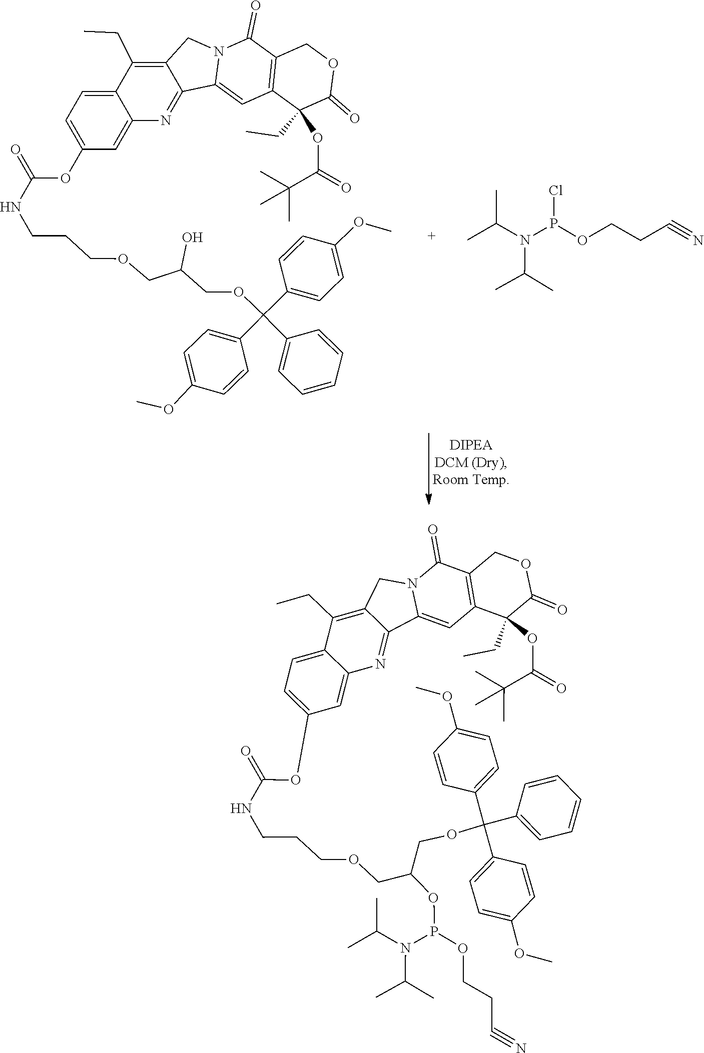 [SMILES: CCc1c2c(nc3cc(OC(=O)NCCCOCC(O)COC(c4ccccc4)(c4ccc(OC)cc4)c4ccc(OC)cc4)ccc13)-c1cc3c(c(=O)n1C2)COC(=O)[C@@]3(CC)OC(=O)C(C)(C)C ; CC(C)N(C(C)C)P(Cl)OCCC#N ; CCc1c2c(nc3cc(OC(=O)NCCCOCC(COC(c4ccccc4)(c4ccc(OC)cc4)c4ccc(OC)cc4)OP(OCCC#N)N(C(C)C)C(C)C)ccc13)-c1cc3c(c(=O)n1C2)COC(=O)[C@@]3(CC)OC(=O)C(C)(C)C]

The purified Compound II-18 (portion of the material 0.255, 0.226 mmole theoretical), dried under vacuum for at least 24 hours is dissolved in dichloromethane (2.26 mL), under inert gas blanket, with magnetic stir bar, followed by addition of DIPEA (0.117 g), and then addition of Cl-Phos. (0.107 g). The reaction is allowed to mix for approximately 15 minutes and then sampled for TLC analysis. When reaction completion is verified, the reaction mixture is washed by adding directly to sodium bicarbonate (2.26 mL, saturated aq.) and organic phase separated, repeated one time. The organic phases are combined and dried over sodium sulfate, anhydrous, and then the sodium sulfate filtered off. The product containing organic phase is sampled for TLC and LC-UV/MS analysis. Then, dichloromethane is stripped off by rotary evaporation and proceeded to purification without crude weight. This crude material is then combined with crude material from a small-scale test reaction. The combined crude material is purified by silica gel solid phase extraction, dichloromethane/methanol/triethylamine mobile phase, product containing fractions are pooled (determined by TLC) sampled for TLC and LC-UV/MS analysis. The, mobile phase is striped off by rotary evaporation, and then placed on vacuum line for at least 24 hours to yield Compound II-19. Compound II-25 can also be prepared similarly from starting with Compound II-24.

### Example 9

### Synthesis of Compounds II-22 and II-28

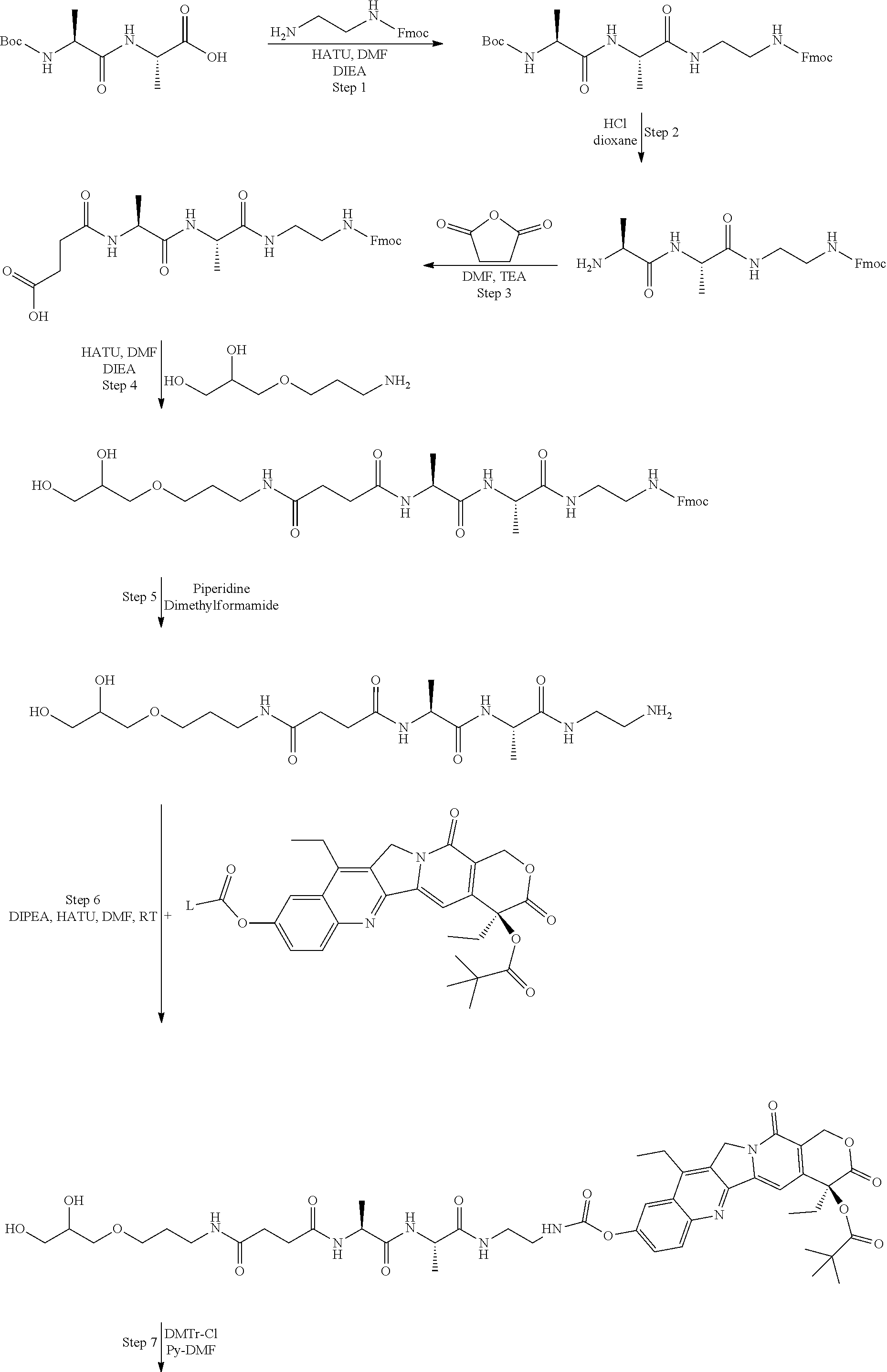

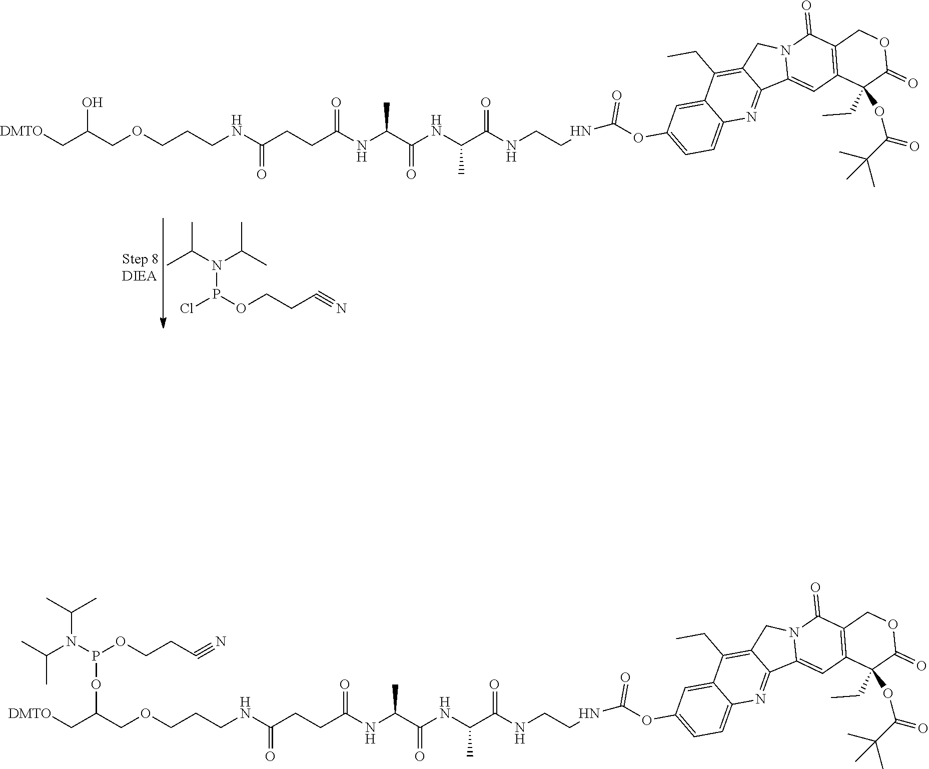

### Step 1

Into a 500 mL round bottomed flask was placed Boc-Ala-Ala-OH (4.0 g, 15.4 mmol, Chem-Impex Cat #04505), DMF (150 mL) and a magnetic stir bar. HATU coupling agent (7.0 g, 18.4 mmol) was added and the mixture was stirred for 5 min before adding Fmoc-1,2-diaminoethane-HCl (4.9 g, 15.4 mmol) and diisopropylethylamine (8.0 mL, 46.1 mmol). After stirring overnight, TLC (silica plats with F254 and dichloromethane:methanol elution 9:1) indicated the reaction was complete. The reaction mixture as concentrated on the rotovap and then partitioned between dichloromethane and water. The solvent layers were separated with an extraction funnel and the aqueous layer extracted three additional times with dichloromethane. The organic layers were combined, dried over sodium sulfate and concentrated under reduced pressure. The product was used in the next step without additional purification.

### Step 2

In a 250 mL round bottomed flask with magnetic stir bar was placed the Boc-Ala-Ala derivative prepared in the previous step (2.5 g, 4.8 mmol). Dichloromethane (30 mL) and DMF (10 mL) were added and the mixture stirred. To this was added 4M hydrochloric acid in dioxane (30 mL, Sigma Cat #345547) and the mixture was stirred. After 1 h, TLC (silica plates with F254, elution with 9:1 dichloromethane:methanol) indicated the reaction was complete. The solvents were removed on the rotovap. Acetonitrile (50 mL) was added and the mixture shaken for a few minutes. The heterogeneous mixture was cooled to 4° C. for 1 h and the solids then collected by filtration (2.0 g). The solids were confirmed to be product by LC-MS.

### Step 3

Into a 250 mL round bottomed flask was placed the amine from the previous step (1.5 g, 3.5 mmol), DMF (35 mL) and a magnetic stir bar. Succinic anhydride (1.8 g, 17.7 mmol) was added in a single portion followed by triethylamine (6.6 mL, 47.2 mmol). The mixture was stirred for 2 h at which point TLC indicated the reaction was complete (TLC elution 4:1 DCM:MeOH). The mixture was concentrated on the rotovap and then treated with potassium carbonate solution (1M, 50 mL). The mixture was allowed to stir for 60 min. The mixture was acidified with hydrochloric acid (20%, 50 mL) and the product precipitated. The mixture was cooled on ice for 30 min and the solids were collected medium frit glass filter. Solid weight 1.32 g. The product was confirmed by LC-MS.

### Step 4

In a small 20 mL glass vial was placed 6,7-dihydroxy-4-oxaheptylamine (443 mg, 3.0 mmol, Berry & Associates Cat #LK4010) and DMF (5 mL). The sample was warmed on a heat plate set a 60° C. for 10 min prior to assembling the other components of the reaction. In a 200 mL round bottomed flask was placed the Succinylated Ala-Ala derivative prepared in the prior step (1.3 g, 2.5 mmol), a magnetic stir bar and DMF (18 mL). HATU (1.1 g, 3.0 mmol, Anaspec) was added and the mixture was stirred for 5 min. To the reaction flask was added with the DMF-amino diol solution in the 20 mL glass vial. The vial was rinsed with 2 mL of DMF and this was added to the reaction vessel. Diisopropylethylamine (1.3 mL, 7.44 mmol) was added, the flask was capped and allowed to stir overnight at room temperature. The magnetic stir bar was removed and the mixture was concentrated on the rotovap and placed on the high vacuum overnight. The reaction was purified on a silica column (Teledyne-Isco) and elution with a dichloromethane:methanol gradient (yield 1.3 g). The product was confirmed by LC-MS.

### Step 5

Fmoc protecting group is cleaved with a base such as piperidine, 4-methylpiperidine, piperazine, 1,8-diazabicyclo[5.4.0]undec-7-ene, or morpholine. A chose of the base depends on how acidic a substrate is resistant towards. The Fmoc protected diol is added to a dried round bottom flask, under inert gas blanket, with magnetic stir bar, followed by addition of DMF. In a separate dried round bottom flask with a magnetic star bar, 20% piperidine in 80% DMF is prepared. To the Fmoc protected diol containing DMF solution, 20% piperidine in 80% DMF is added and the resulting mixture is allowed to mix at under inert gas, at room temperature, until reaction completion verified by TLC and LC-UV/MS analysis. At reaction completion the solvents are stripped off by rotary evaporation, under vacuum (10 mbar), with heating (55° C.). The concentrated residue is placed under full vacuum, at room temperature, for several hours resulting in crude Compound. The product is isolated by flash chromatography 24 g silica column (Teledyne-ISCO) and eluted with a dichloromethane methanol gradient.

### Step 6

Carbonylated Camptothecin derivative prepared according to the above carbonylation is added to a dried round bottom flask, under inert gas blanket, with magnetic stir bar, followed by addition of DMF (6.10 mL). The carbonylated Camptothecin derivative is allowed to dissolve completely under inert gas at room temperature. Then, to the mixture is added DIPEA (0.351 g, 2.013 mmole), followed by addition of HATU (0.278 g, 0.732 mmole). The amine diol (0.610 mmole) is added to a dried round bottom flask, under inert gas blanket, with magnetic stir bar, followed by addition of DMF (6.10 mL), and dissolve completely at room temperature. Then the carbonylated Camptothecin derivative reaction mixture is added to the solution containing the amine diol; the resultant mixture is allowed to mix at under inert gas, at room temperature, until reaction completion verified by TLC and LC-UV/MS analysis.

At reaction completion the solvents are stripped off by rotary evaporation, under vacuum (10 mbar), with heating (55° C.). The concentrated residue is placed under full vacuum, at room temperature, for several hours resulting in crude Compound II-20. Compound II-26 can also be prepared similarly with the camptothecin derivative with a hydroxyl group at 11 position instead of 10 position.

### Step 7

In a 200 mL round bottomed flask with magnetic stir bar is placed the diol prepared in the prior step (1.9 mmol) and pyridine (38 mL). 4,4-dimethoxytrityl chloride is added to the solution in a single portion. The flask is capped and allowed to stir overnight. The mixture is concentrated under reduced pressure and the product is isolated by flash chromatography 24 g silica column (Teledyne-ISCO) and eluted with a dichloromethane:methanol gradient resulting in Compound II-21. Compound II-27 can also be prepared similarly.

### Step 8

The tritylated product is placed in 50 mL RB flask with magnetic stir bar (0.28 mmol). Dichloromethane (5.5 mL) and 4 to 5.4 Å molecular sieves are added. 2-Cyanoethyl N,N-diisopropylchlorophosphoramidite (192 μL, 0.6 mmol) and diisopropylethylamine (192 μL, 1.1 mmol) are added via syringe. The reaction is stirred for 2 h. The reaction is monitored by TLC (100% DCM with 5% TEA-prewashed TLC plates). The reaction is concentrated and taken up on silica gel (12 g column—Teledyne-ISCO) and eluted with a hexane:ethylacetate gradient with 2.5% trimethylamine resulting in Compound II-22. Compound II-28 can also be prepared similarly.

### Example 10

### Activation and Antibody Conjugation of Compound I-29 Leading to a Formulation of Compound I-29 ADC

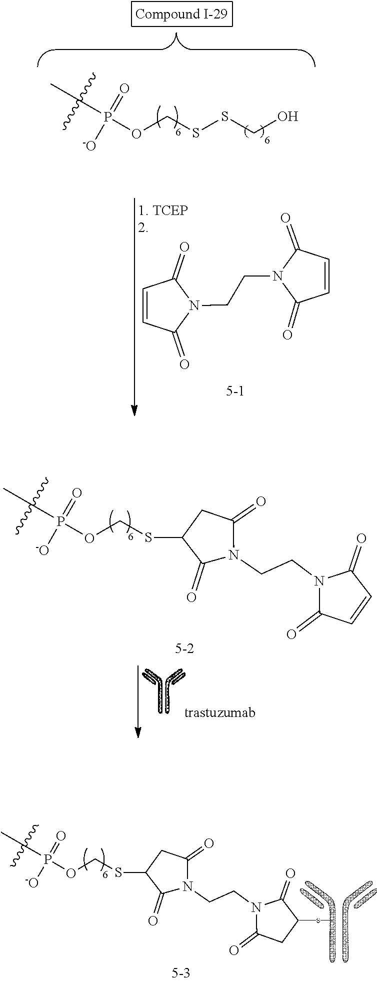

The thiol protecting group of Compound I-29 was removed using standard reducing conditions (i.e., TCEP) and the deprotected thiol was functionalized with 5-1 (bis-maleimidoethane; “BMOE”) to afford 5-2. In parallel, a trastuzumab antibody was treated with BMOE to reduce disulfide bonds. The reduced trastuzumab antibody was reacted with 5-2 (1.5 g) in a 5:1 molar ratio of polymer to antibody. The reaction resulted in a final product 5-3 (i.e., compound I-29 ADC). The following ADCs were synthesized according to the above procedures:

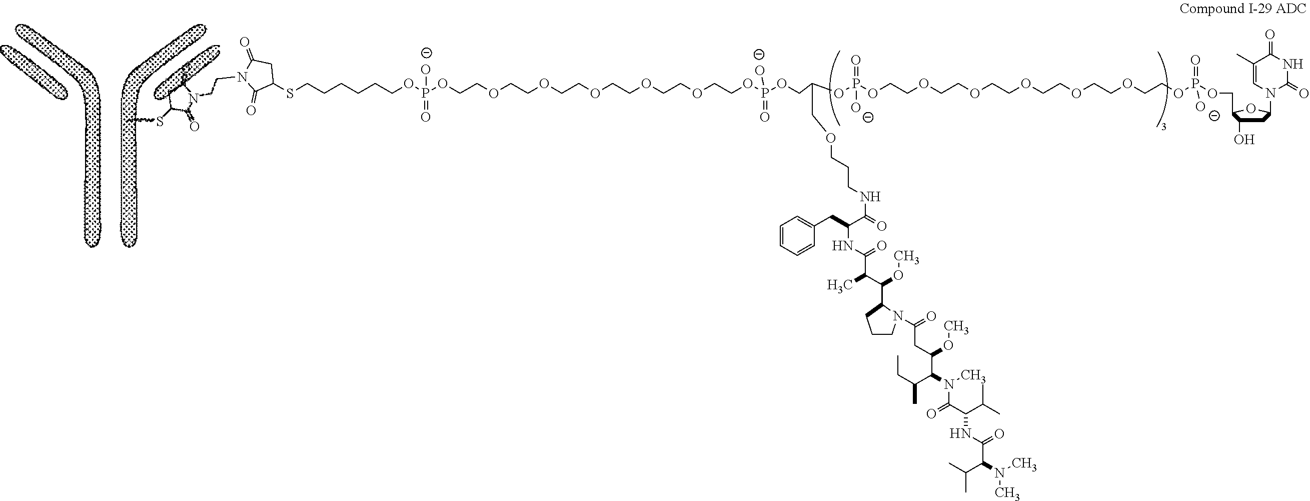

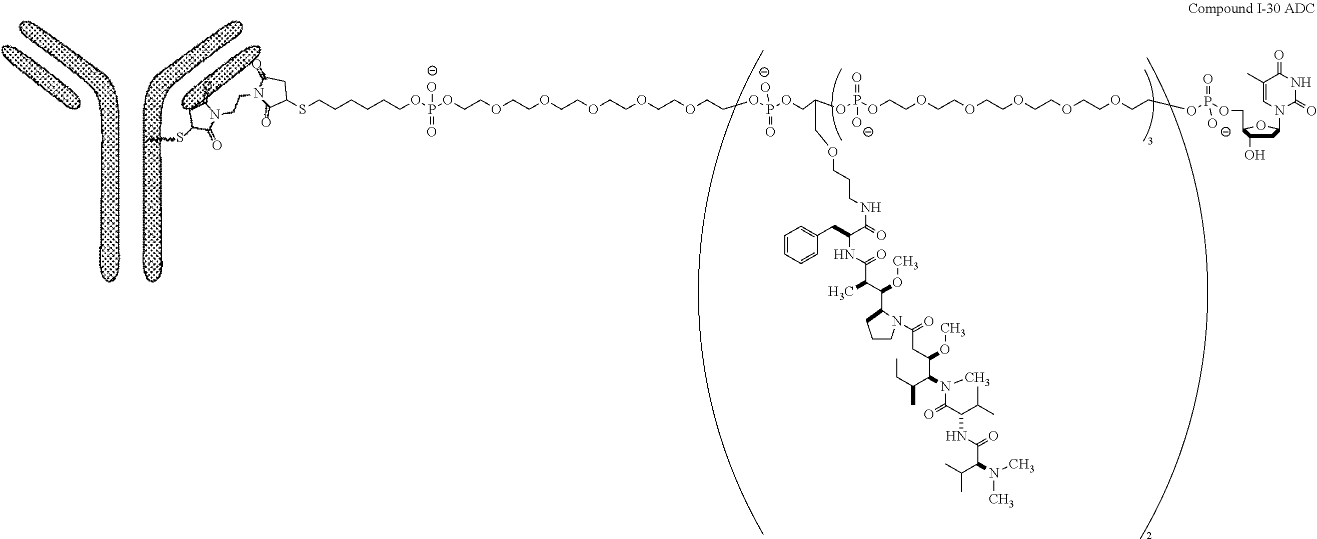

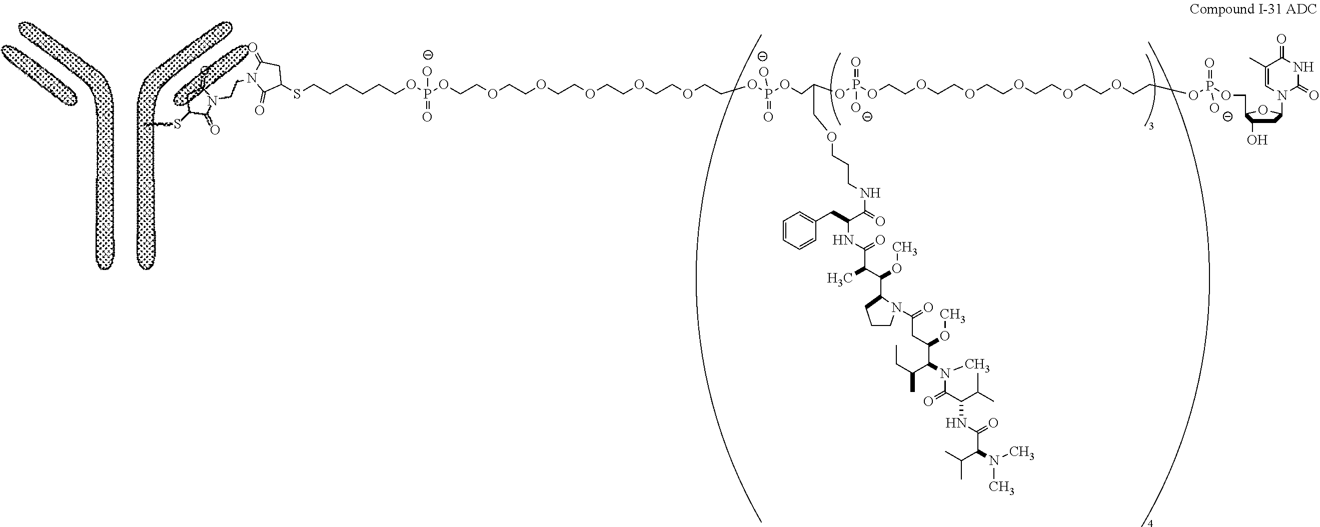

Other compounds disclosed herein (I-1-I-37) can be conjugated with antibody by the same method described above to generate other ADCs. Although antibody trastuzumab is used in EXAMPLE 10 demonstrating conjugation between compounds disclosed herein and an antibody, it is used for merely an illustration and can include other antibodies such as brentuximab, gemtuzumab, trastuzumab, inotuzumab, polatuzumab, enfortumab, trastuzumab, sacituzumab, belantamab, or moxetumomab.

### Example 11

### Formation of Cysteine Quenched Compound I-29 Used for Stability Study

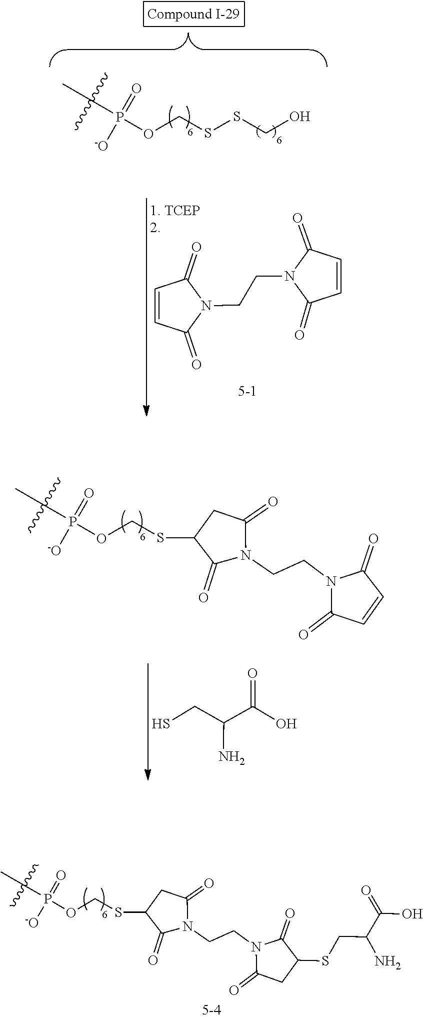

The thiol protecting group of Compound I-29 was removed using standard reducing conditions (i.e., TCEP) and the deprotected thiol was functionalized with 5-1 (bis-maleimidoethane; “BMOE”) to afford 5-2. In order to mimic an ADC, the maleimide moiety of compounds was quenched with cysteine to form thiosuccinimide which is the moiety generated by the reaction with an antibody. Thus, cysteine was reacted with 5-2. The reaction resulted in a final product 5-3 (i.e., cysteine quenched compound I-29). The following ADCs were synthesized according to the above procedures:

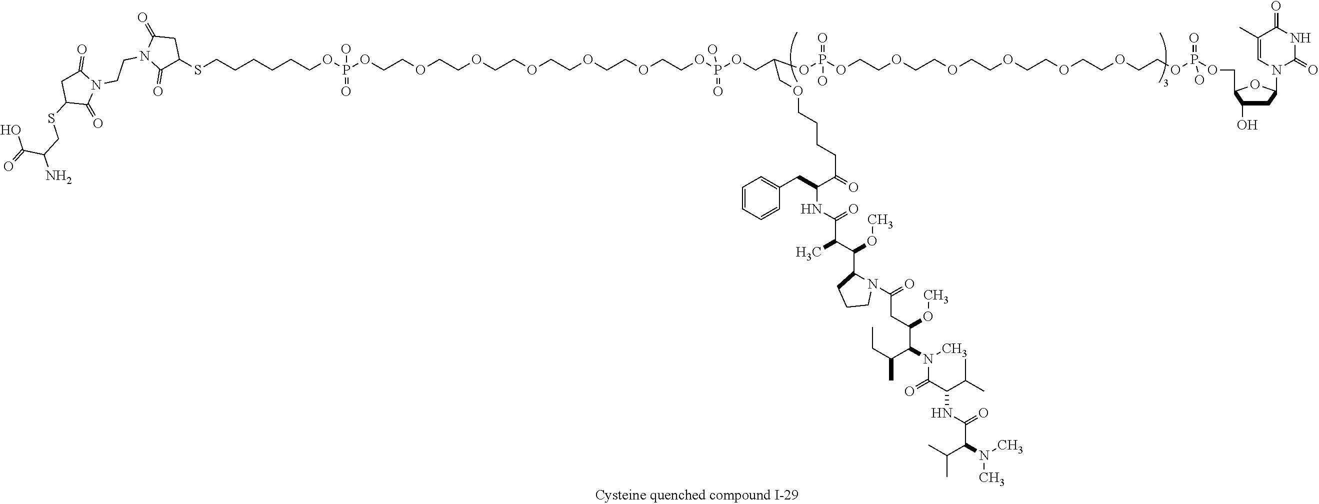

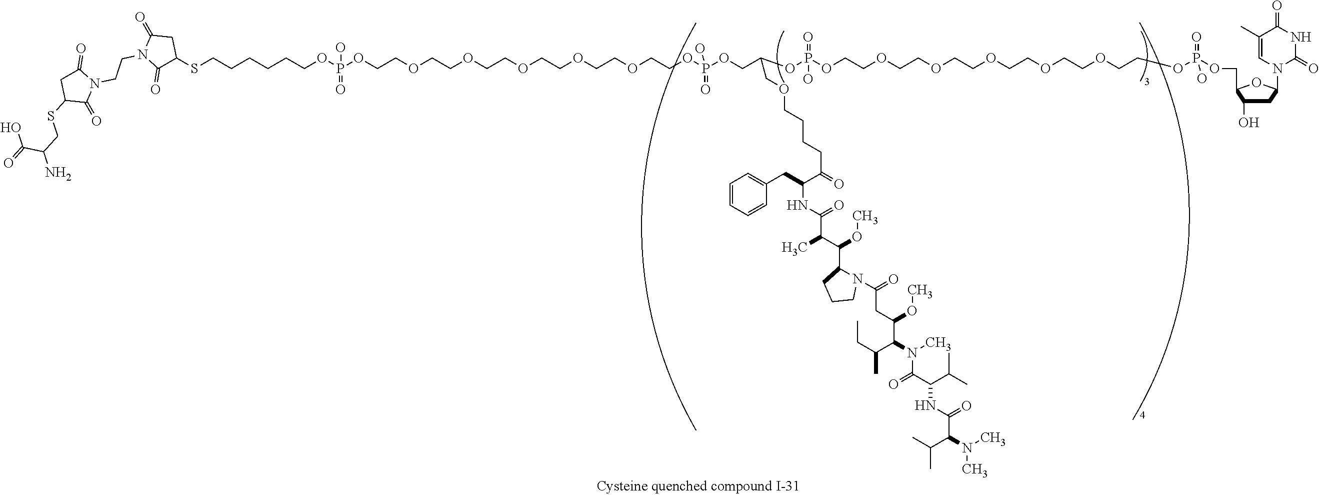

### In-Vitro Stability Study

In-vitro stability of compounds disclosed herein was studied. The samples for the in-vitro stability study used include ADC compounds (antibody-polymer-drug), polymer-drug, and antibody only. The antibody used in this stability study was Trastuzumab. The incubation medium were phosphate buffered saline (PBS), Human plasma, and mouse plasma, and the incubation temperatures included 4, 32, and 37° C. The stability was tested at day 0 (i.e., start of the experiment), d, 3, 4, 7, 14, and 28 using an analytical size exclusion chromatography (SEC) to detect amount of ADC present, amount of large molecular weight breakdown, and amount of aggregate. Additionally, an analytical liquid chromatography (LC) was used to detect stability of linkers and breakdown of polymer/drug.

### Example 12

### Cell Proliferation Assay

Compound I-31 was prepared from Auristatin F-phosphoramidite and prepared on the DNA synthesizer as disclosed in the present disclosure. Compound I-31 was activated and conjugated to the commercial antibody Trastuzumab. As shown in the Graphs 1-2 below, compound I-31 ADC (labeled as “ADC-4×”) was more potent and cytotoxic than its component parts including cysteine quenched I-31 (labeled as “Poly-AF-4×”), Auristatin F alone (labeled as “AF”), or Trastuzumab (labeled as “Herceptin”). Compound I-31 ADC was found to be potent and selective versus cell lines expressing the Her2 antigen.

### Example 13

### Stability of Cysteine Quenched Compounds I-29, I-30, I-31, and the Corresponding ADCs

Analytical SEC: An Agilent 1260 Infinity II separations module equipped with Agilent UV photodiode array detector (G7115A), Multisampler (G5668A), column heater (G7116A) and Bio inert Quant Pump (G5654A) was used. It was outfitted with a Tosoh Biosciences G3000SXWL 7.8 mm×30 cm, 5 micron, (part #08511). The column temperature was set at 30° C. An isocratic elution was used with a running buffer consisting of PBS buffer pH=7.4. The flow rate was set at 0.5 mL/min.

Approximately 1-20 ug of sample was injected per analysis. Detection was set at and 215, 280 nm and 498 nm. The method is run for 20 minutes. Quantitation was done with Agilent Chemstation software. Trastuzumab and compound I-29 were reacted according to the above procedure to generate compound I-29 ADC which showed good stability at 100 μg/mL in PBS over 7 days at 37° C. with aggregate content up to 5%±2%. At 32° C., compound I-29 ADC exhibited minimal change over 14 days period with up to 5%±2%.

Aggregate content with compound I-29 ADC was studied with an interval of 0, 7, 14, and 28 days at 37° C. in PBS buffer at 100 μg/mL. The result is shown in the graph below.

Similarly, Trastuzumab and compound I-30 were reacted according to the above procedure to generate compound I-30 ADC and tested for the in-vitro stability, which showed good stability at 100 μg/mL in PBS over 7 days at 37° C. with aggregate content up to 7.5%±2%. At 32° C., compound I-30 ADC exhibited minimal change over 14 days period with up to 7%±3%. The degree of labeling (DOL) of antibodies was approximately 2.5.

Trastuzumab and compound I-31 were reacted according to the above procedure to generate compound I-31 ADC and tested for the in-vitro stability, which showed good stability at 100 μg/mL in PBS over 7 days at 37° C. with aggregate content up to 4%±1%.

A control study with Trastuzumab alone was conducted at 100 μg/mL in PBS at both 32° C. and 37° C. Both at 32° C. or 37° C., the stability has not changed over 14 days and the aggregate content was found to be less than 2%.

Another set of control study with the cysteine quenched compound I-29 was conducted at 100 μg/mL in PBS at 32° C. Compound I-29 was treated with cysteine to quench maleimide as depicted above. At 32° C., the aggregate content was 8%±3% after 14 days. Similarly, another set of control study with the cysteine quenched compound I-31 was conducted at 100 μg/mL in PBS at 32° C. Compound I-31 was treated with cysteine to quench maleimide before the study to mimic ADC without antibody. At 32° C., the aggregate content was 15%±3% after 14 days.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Provisional Application No. 63/224,777, filed Jul. 22, 2021; U.S. Provisional Application No. 63/250,892, filed Sep. 30, 2021; and U.S. Provisional Application No. 63/252,993, filed Oct. 6, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following Structure (I): 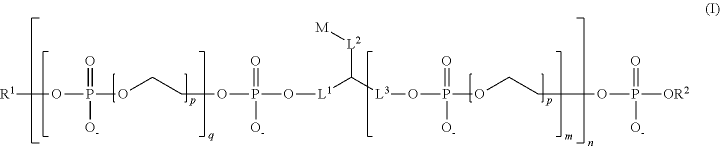
or a pharmaceutically acceptable salt, stereoisomer, or tautomer thereof, wherein
R¹ and R² are independently hydrogen, alkyl, heteroalkyl, Q or a protected form thereof, L′, or have the following structure: 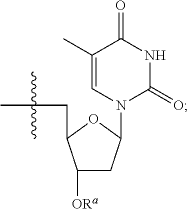
R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0;
p is an integer greater than 0; and
q is an integer greater than 0.

2. A compound having the following Structure (I): 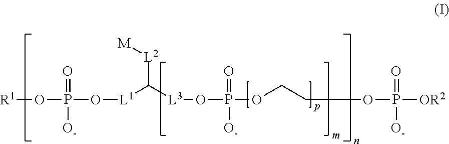
or a pharmaceutically acceptable salt, stereoisomer, or tautomer thereof, wherein
R¹ and R² are independently hydrogen, alkyl, heteroalkyl, Q or a protected form thereof, L′, or have the following structure: 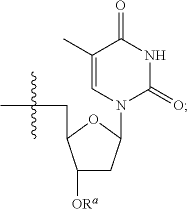
R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0; and
p is an integer greater than 0.

3. The compound of any one of claims 1-2, wherein R¹ is L′.

4. The compound of any one of claims 1-3, wherein L′ is a linker to a targeting moiety.

5. The compound of any one of claims 1-4, wherein L′ is a linker to a targeting moiety, the linker comprising an alkylene oxide or phosphodiester moiety, or combinations thereof.

6. The compound of any one of claims 3-5, wherein L′ has one of the following structures: 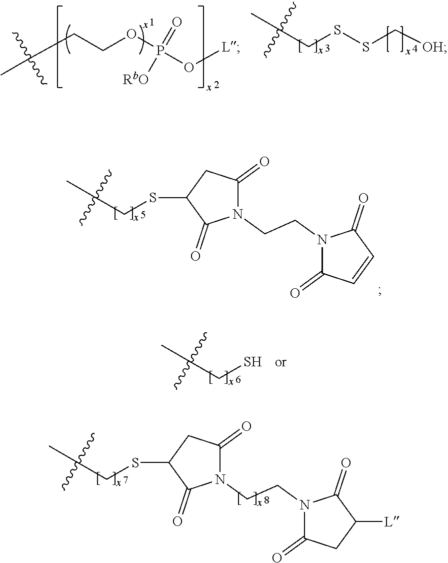
wherein:
x¹, x², x³, x⁴, x⁶, x⁷ and x⁸ are independently an integer from 1 to 10;
R^{b} is H, an electron pair or a counter ion;
L″ is the targeting moiety or a linkage to the targeting moiety.

7. The compound of any one of claims 1-6, wherein the targeting moiety is an antibody or cell surface receptor antagonist.

8. The compound of claim 7, wherein the antibody or cell surface receptor antagonist is an epidermal growth factor receptor (EGFR) inhibitor, a hepatocyte growth factor receptor (HGFR) inhibitor, an insulin-like growth factor receptor (IGFR) inhibitor, a folate, or a MET inhibitor.

9. The compound of any one of claims 1-8, wherein R¹ or R² has one of the 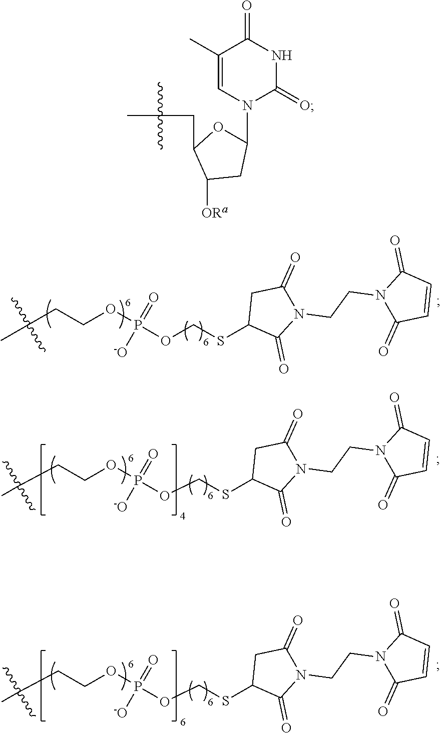 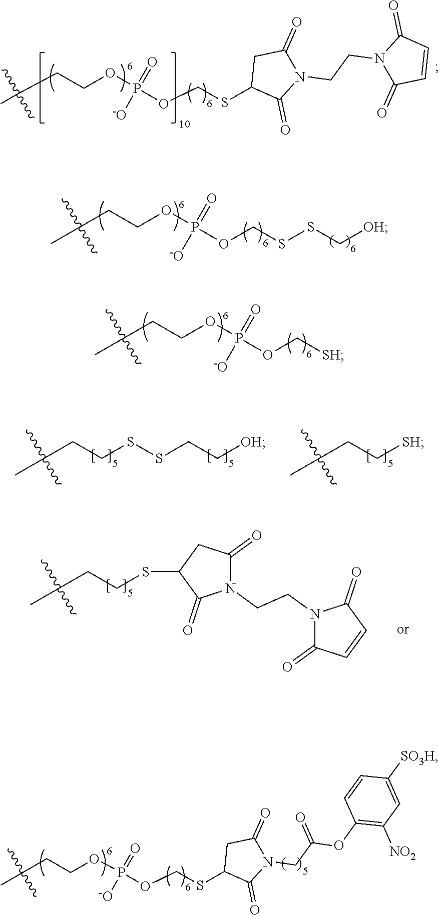
wherein
R^{a} is H or a solid support.

10. The compound of any one of claims 1-9, wherein R¹ has one of the following structures:

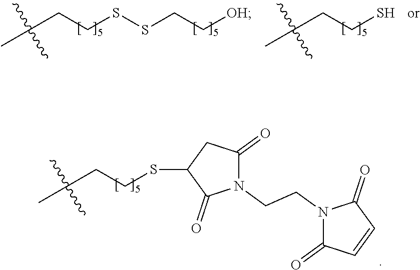

11. The compound of any one of claims 1-10, wherein R² has the following structure:

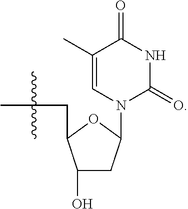 [SMILES: *CC1OC(n2cc(C)c(=O)[nH]c2=O)CC1O]

12. The compound of any one of claims 1-11, wherein at least one occurrence of L¹ is alkylene.

13. The compound of any one of claims 1-12, wherein at least one occurrence of L¹ is C₁-C₆ alkylene.

14. The compound of any one of claims 1-13, wherein at least one occurrence of L¹ is methylene.

15. The compound of any one of claims 1-14, wherein each occurrence of L¹ is alkylene.

16. The compound of any one of claims 1-15, wherein each occurrence of L¹ is C₁-C₆ alkylene.

17. The compound of any one of claims 1-16, wherein each occurrence of L¹ is methylene.

18. The compound of any one of claims 1-17, wherein at least one occurrence of L³ is alkylene.

19. The compound of any one of claims 1-18, wherein at least one occurrence of L³ is C₁-C₆ alkylene.

20. The compound of any one of claims 1-19, wherein at least one occurrence of L³ is methylene.

21. The compound of any one of claims 1-20, wherein each occurrence of L³ is alkylene.

22. The compound of any one of claims 1-21, wherein each occurrence of L³ is C₁-C₆ alkylene.

23. The compound of any one of claims 1-22, wherein each occurrence of L³ is methylene.

24. The compound of any one of claims 1-20, wherein at least one occurrence of L³ is a direct bond.

25. The compound of any one of claims 1-17, wherein each occurrence of L³ is a direct bond.

26. The compound of any one of claims 1-25, wherein at least one occurrence of L² is heteroalkylene.

27. The compound of any one of claims 1-26, wherein at least one occurrence of L² comprises oxygen.

28. The compound of any one of claims 1-27, wherein at least one occurrence of L² has the following structure: 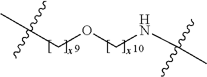
wherein:
x⁹ and x¹⁰ are each independently an integer greater than 0.

29. The compound of claim 28, wherein x⁹ is 1, 2, 3, or 4.

30. The compound of claim 28 or 29, wherein x¹⁰ is 2, 3, 4, or 5.

31. The compound of any one of claims 28-30, wherein x⁹ is 1 or 2 and x¹⁰ is 2, 3, or 4.

32. The compound of any one of claims 1-31, wherein each occurrence of L² is heteroalkylene.

33. The compound of any one of claims 1-32, wherein each occurrence of L² comprises oxygen.

34. The compound of any one of claims 1-33, wherein each occurrence of L² has the following structure: 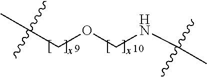
wherein:
x⁹ and x¹⁰ are each independently an integer greater than 0.

35. The compound of claim 34, wherein x⁹ is 1, 2, 3, or 4.

36. The compound of claim 34 or 35, wherein x¹⁰ is 2, 3, 4, or 5.

37. The compound of any one of claims 34-36, wherein x⁹ is 1 or 2 and x¹⁰ is 2, 3, or 4.

38. The compound of claim 28, wherein L² further comprises a physiologically cleavable linker.

39. The compound of claim 38, wherein at least one occurrence of L² comprises an amide bond, an ester bond, a phosphodiester bond, a disulfide bond, a double bond, a triple bond, an ether bond, a hydrazone, an amino acid sequence comprising one or more amino acid residues, a ketone, a diol, a cyano, a nitro, or combinations thereof.

40. The compound of claim 38 or 39, wherein at least one occurrence of L² comprises an amino acid sequence recognized by a sortase enzyme or cysteine protease.

41. The compound of claim 40, wherein the amino acid sequence is Leu-Pro-X-Thr-Gly, wherein X is any amino acid residue.

42. The compound of any one of claims 1-41, wherein at least one occurrence of L² comprises one of the following structures:

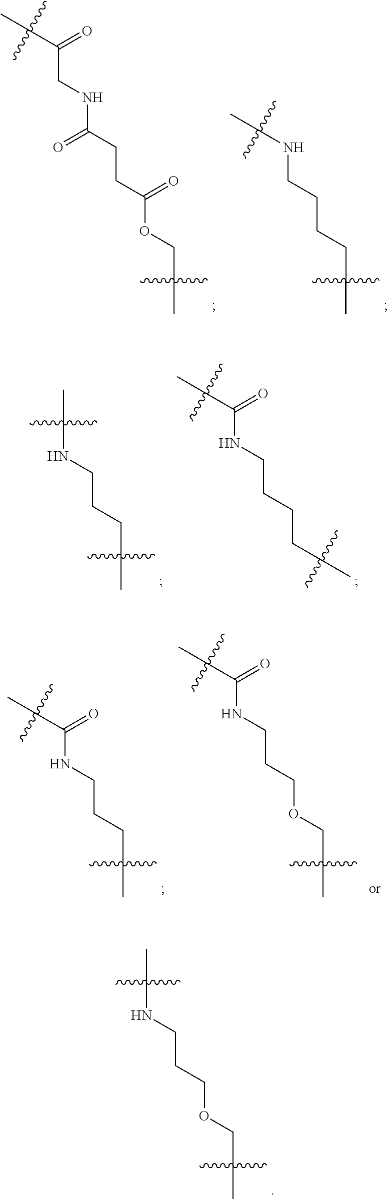

43. The compound of any one of claims 38-42, wherein each occurrence of L² comprises an amide bond, an ester bond, a phosphodiester bond, a disulfide bond, a double bond, a triple bond, an ether bond, a hydrazone, an amino acid sequence, a ketone, a diol, a cyano, a nitro or combinations thereof.

44. The compound of any one of claims 38-43, wherein each occurrence of L² comprises one of the following structures:

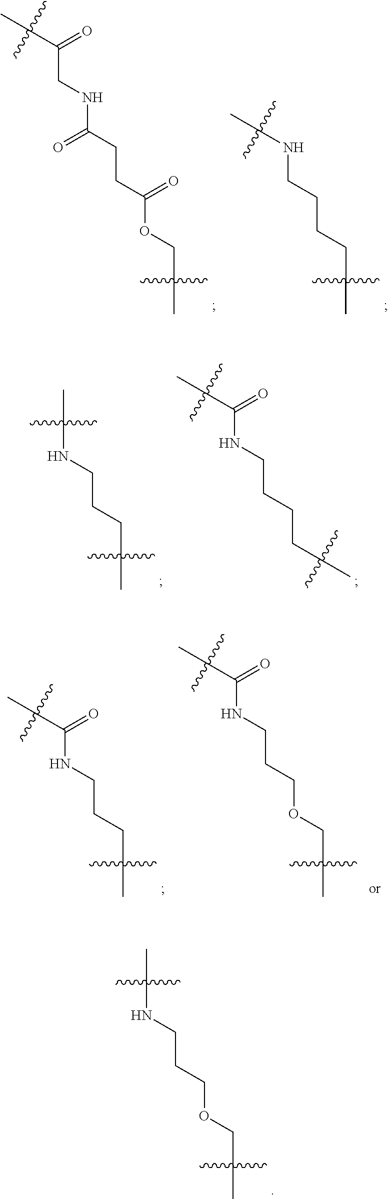

45. The compound of any one of claims 38-44, wherein at least one occurrence of L² comprises one or more amino acid residues.

46. The compound of claim 45, wherein at least one occurrence of L² comprises one or more amino acid residues selected from the group consisting of alanine, valine, and combinations thereof.

47. The compound of any one of claims 38-46, wherein at least one occurrence of L² comprises one of the following structures:

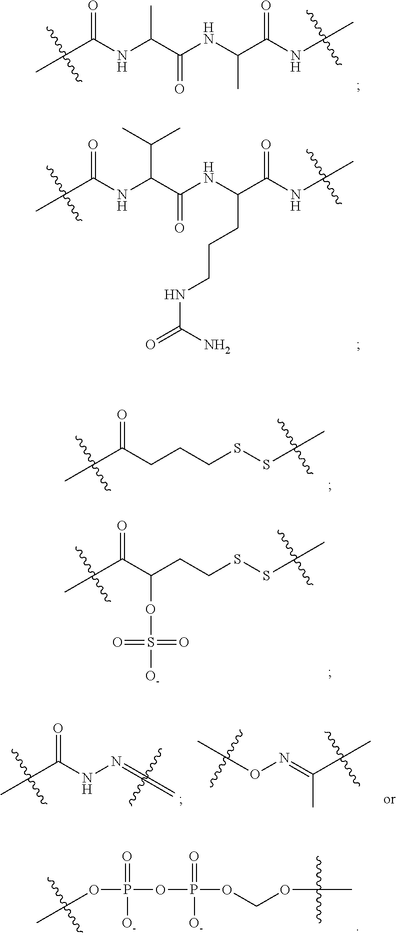

48. The compound of any one of claims 38-47, wherein each occurrence of L² comprises one or more amino acid residues.

49. The compound of claim 48, wherein each occurrence of L² comprises one or more amino acid residues selected from the group consisting of alanine, valine, and combinations thereof.

50. The compound of any one of claims 38-49, wherein each occurrence of L² comprises one of the following structures:

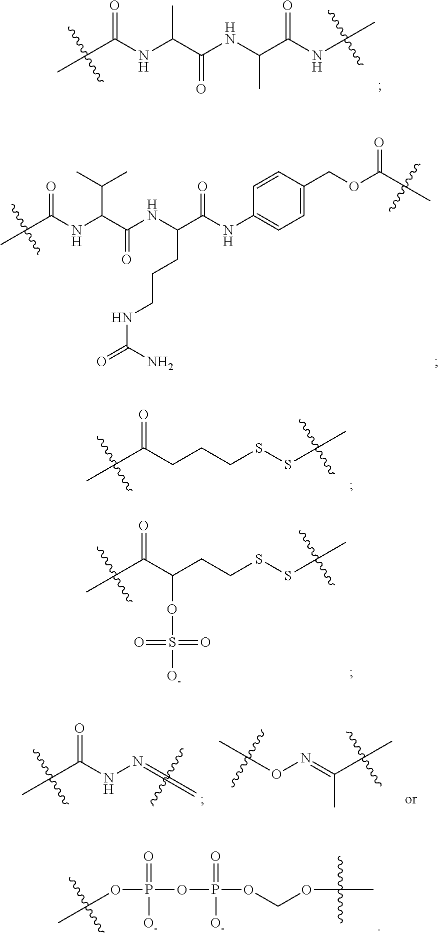

51. The compound of any one of claims 1-50, wherein at least one occurrence of L² has one of the following structures:

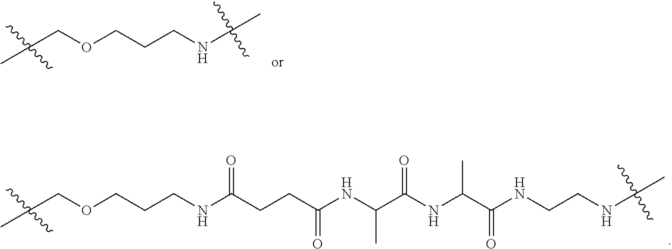 [SMILES: *COCCCN* ; *COCCCNC(=O)CCC(=O)NC(C)C(=O)NC(C)C(=O)NCCN*]

52. The compound of any one of claims 1-51, wherein each occurrence of L² has one of the following structures:

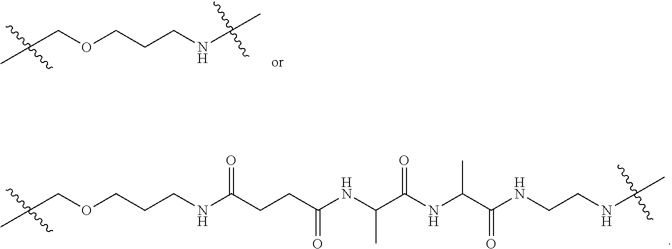 [SMILES: *COCCCN* ; *COCCCNC(=O)CCC(=O)NC(C)C(=O)NC(C)C(=O)NCCN*]

53. The compound of any one of claims 1-52, wherein at least one occurrence of M is an alkylating agent, an antimetabolite, a microtubule inhibitor, a topoisomerase inhibitor, or a cytotoxic antibiotic.

54. The compound of any one of claims 1-53, wherein each occurrence of M is an alkylating agent, an antimetabolite, a microtubule inhibitor, a topoisomerase inhibitor, or a cytotoxic antibiotic.

55. The compound of any one of claims 1-54, wherein at least one occurrence of M is an alkylating agent, an antimetabolite, a microtubule inhibitor, or a topoisomerase inhibitor.

56. The compound of any one of claims 1-55, wherein each occurrence of M is an alkylating agent, an antimetabolite, a microtubule inhibitor, or a topoisomerase inhibitor.

57. The compound of any one of claims 1-56, wherein at least one occurrence of M is a nitrogen mustard, a nitrosourea, a tetrazine, an aziridine, a cisplatin or cisplatin derivative, or a non-classical alkylating agent.

58. The compound of any one of claims 1-57, wherein at least one occurrence of M is mechlorethamine, cyclophosphamide, melphalan, chlorambucil, ifosfamide, busulfan, N-nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine, or hexamethylmelamine.

59. The compound of any one of claims 1-58, wherein at least one occurrence of M is an anti-folate, a fluoropyrimidines, a deoxynucleoside analogue, or a thiopurine.

60. The compound of any one of claims 1-59, wherein at least one occurrence of M is methotrexate, pemetrexed, fluorouracil, capecitabine, cytarabine, gemcitabine, decitabine, azacitidine, fludarabine, nelarabine, cladribine, clofarabine, pentostatin, thioguanine, and mercaptopurine.

61. The compound of any one of claims 1-60, wherein at least one occurrence of M is an auristatin, a Vinca alkaloid, or a taxane.

62. The compound of any one of claims 1-61, wherein at least one occurrence of M is auristatin F, auristatin E, vincristine, vinblastine, vinorelbine, vindesine, vinflunine, paclitaxel, docetaxel, etoposide, or teniposide.

63. The compound of any one of claims 1-62, wherein at least one occurrence of M is irinotecan, SN 38, topotecan, camptothecin, doxorubicin, mitoxantrone, teniposide. novobiocin, merbarone, or aclarubicin.

64. The compound of any one of claims 1-63, wherein at least one occurrence of M is an anthracycline or a bleomycin.

65. The compound of any one of claims 1-64, wherein at least one occurrence of M is doxorubicin, daunorubicin, epirubicin, idarubicin, pirarubicin, aclarubicin, or mitoxantrone.

66. The compound of any one of claims 1-65, wherein at least one occurrence of M is a camptothecin.

67. The compound of any one of claims 1-66, wherein at least one occurrence of M has the following structure:

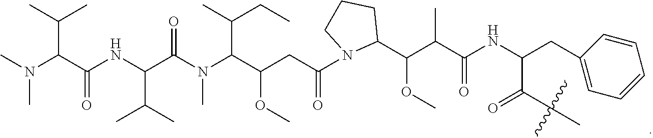 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

68. The compound of any one of claims 1-56, wherein each occurrence of M has the following structure:

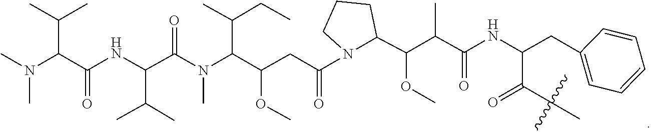 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

69. The compound of any one of claims 1-67, wherein at least one occurrence of M has the following structure:

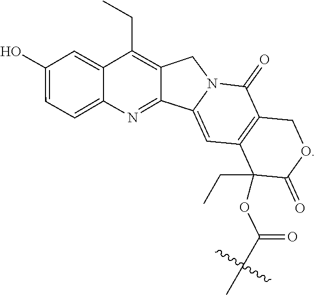 [SMILES: *C(=O)OC1(CC)C(=O)OCc2c1cc1n(c2=O)Cc2c-1nc1ccc(O)cc1c2CC]

70. The compound of any one of claims 1-56, wherein each occurrence of M has the following structure:

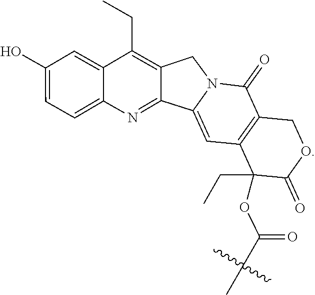 [SMILES: *C(=O)OC1(CC)C(=O)OCc2c1cc1n(c2=O)Cc2c-1nc1ccc(O)cc1c2CC]

71. The compound of any one of claims 1-56, wherein at least one occurrence of -L²-M has one of the following structures:

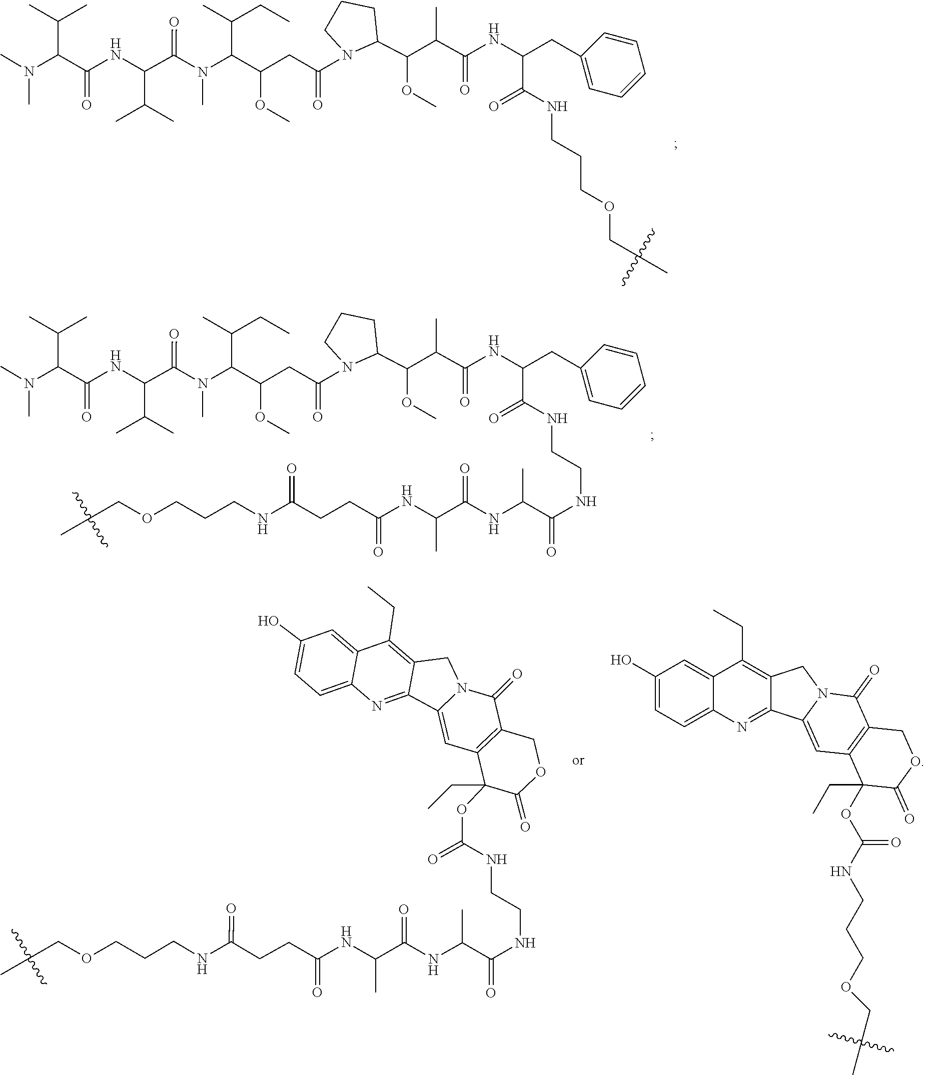

72. The compound of any one of claims 1-56, wherein each occurrence of -L²-M has one of the following structures:

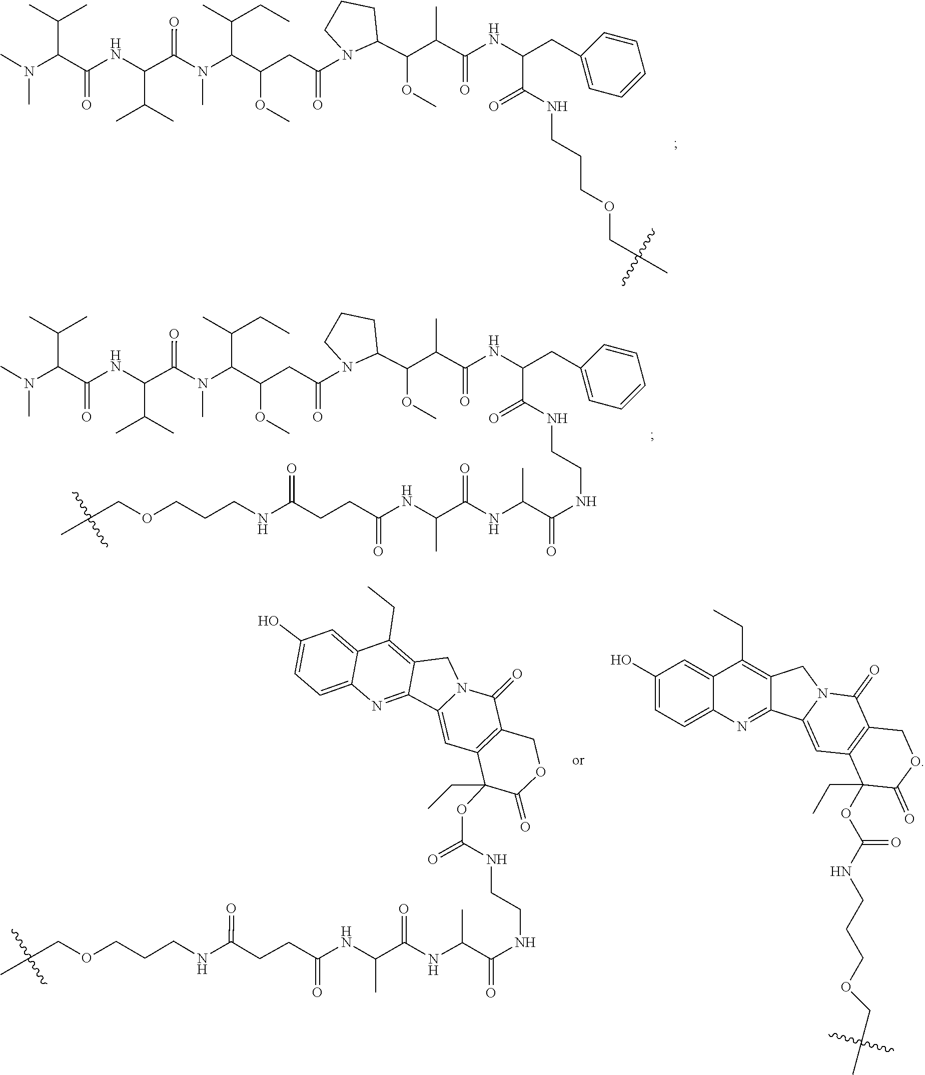

73. The compound of any one of claims 1-72, wherein M has one of the following structures: 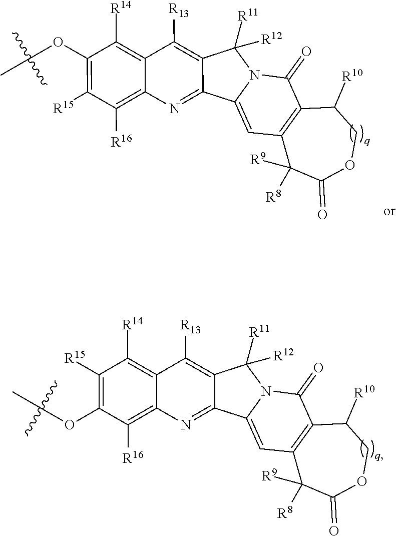
wherein:
R⁸ is, at each occurrence, independently H or OH;
R⁹, R¹⁰, R¹¹, and R¹² are, at each occurrence, independently H, linear or branched alkyl, linear or branched alkyaryl, hydroxyalkyl, or aryl;
R¹³, R¹⁴, R¹⁵, and R¹⁶ are, at each occurrence, independently H, linear or branched alkyl, linear or branched alkyaryl, carbonyl, alkoxy, aryloxy, hydroxyalkyl, nitro, cyano, or aminoalkoxy; and
q is 0 or 1.

74. The compound of any one of claims 1-73, wherein at least one occurrence of M has the following structure:

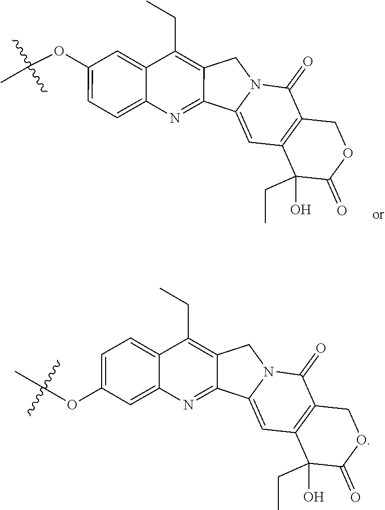 [SMILES: *Oc1ccc2nc3c(c(CC)c2c1)Cn1c-3cc2c(c1=O)COC(=O)C2(O)CC ; *Oc1ccc2c(CC)c3c(nc2c1)-c1cc2c(c(=O)n1C3)COC(=O)C2(O)CC]

75. The compound of any one of claims 1-56, wherein each occurrence of M has the following structure:

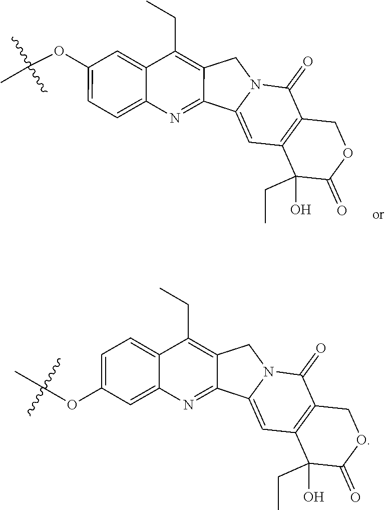 [SMILES: *Oc1ccc2nc3c(c(CC)c2c1)Cn1c-3cc2c(c1=O)COC(=O)C2(O)CC ; *Oc1ccc2c(CC)c3c(nc2c1)-c1cc2c(c(=O)n1C3)COC(=O)C2(O)CC]

76. The compound of any one of claims 1-75, wherein at least one occurrence of -L²-M has one of the following structures:

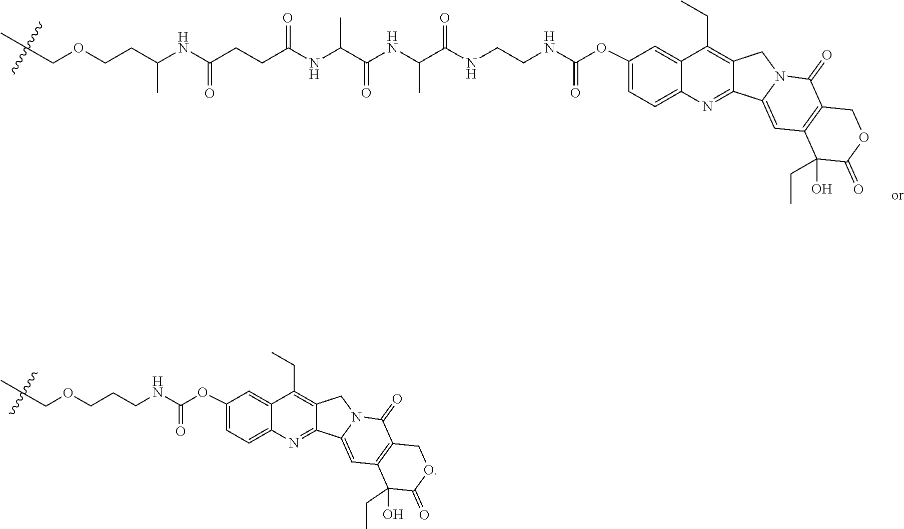 [SMILES: *COCCC(C)NC(=O)CCC(=O)NC(C)C(=O)NC(C)C(=O)NCCNC(=O)Oc1ccc2nc3c(c(CC)c2c1)Cn1c-3cc2c(c1=O)COC(=O)C2(O)CC ; *COCCCNC(=O)Oc1ccc2nc3c(c(CC)c2c1)Cn1c-3cc2c(c1=O)COC(=O)C2(O)CC]

77. The compound of any one of claims 1-56, wherein each occurrence of -L²-M has one of the following structures:

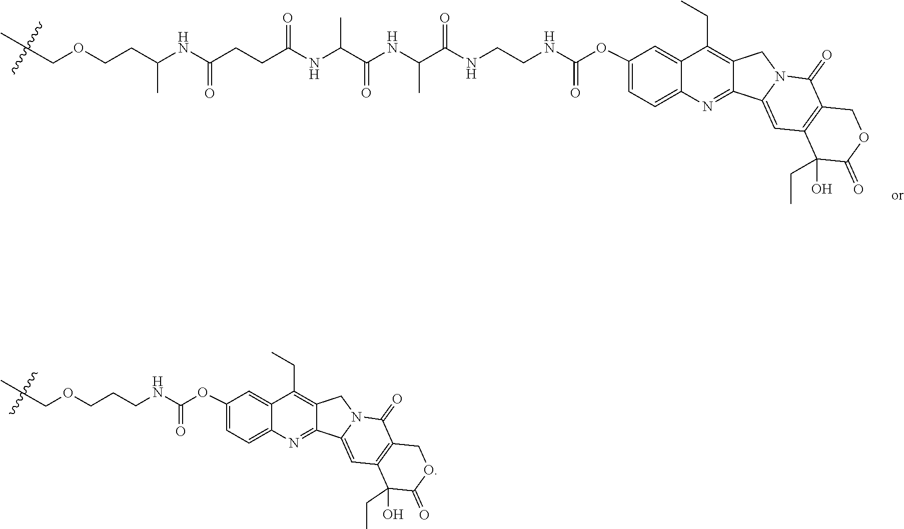 [SMILES: *COCCC(C)NC(=O)CCC(=O)NC(C)C(=O)NC(C)C(=O)NCCNC(=O)Oc1ccc2nc3c(c(CC)c2c1)Cn1c-3cc2c(c1=O)COC(=O)C2(O)CC ; *COCCCNC(=O)Oc1ccc2nc3c(c(CC)c2c1)Cn1c-3cc2c(c1=O)COC(=O)C2(O)CC]

78. The compound of any one of claims 1-77, wherein at least one occurrence of -L²-M has one of the following structures:

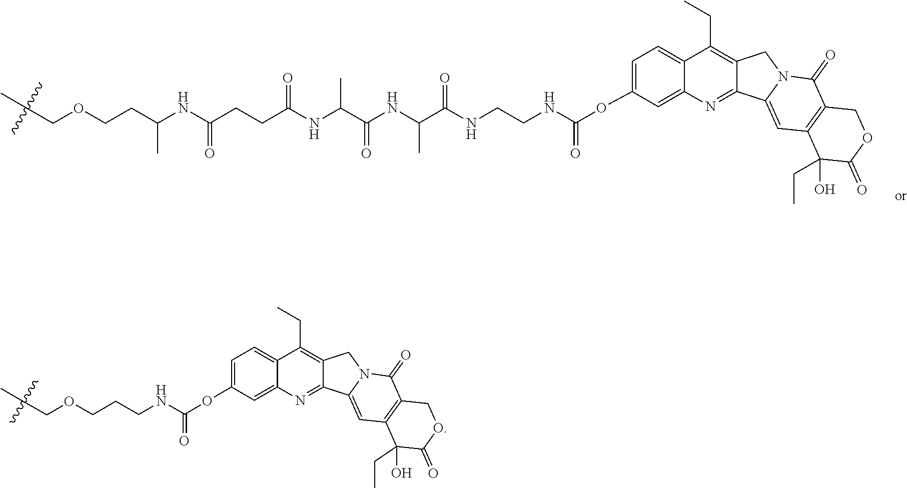 [SMILES: *COCCC(C)NC(=O)CCC(=O)NC(C)C(=O)NC(C)C(=O)NCCNC(=O)Oc1ccc2c(CC)c3c(nc2c1)-c1cc2c(c(=O)n1C3)COC(=O)C2(O)CC ; *COCCCNC(=O)Oc1ccc2c(CC)c3c(nc2c1)-c1cc2c(c(=O)n1C3)COC(=O)C2(O)CC]

79. The compound of any one of claims 1-56, wherein each occurrence of -L²-M has one of the following structures:

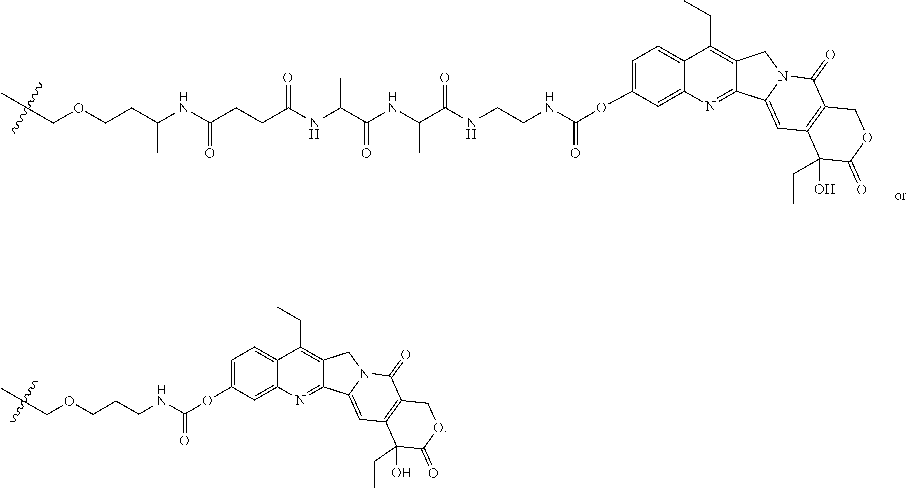 [SMILES: *COCCC(C)NC(=O)CCC(=O)NC(C)C(=O)NC(C)C(=O)NCCNC(=O)Oc1ccc2c(CC)c3c(nc2c1)-c1cc2c(c(=O)n1C3)COC(=O)C2(O)CC ; *COCCCNC(=O)Oc1ccc2c(CC)c3c(nc2c1)-c1cc2c(c(=O)n1C3)COC(=O)C2(O)CC]

80. The compound of any one of claims 1-79, wherein n is 1, 2, 3, 4, 5, or 6.

81. The compound ofany one of claims 1-80, wherein n is 1, 2, 3, or 4.

82. The compound of any one of claims 1-81, wherein the compound has one of the structures of Table 1 or a salt or tautomer thereof.

83. A pharmaceutical composition comprising the compound of any one of claims 1-82, and a pharmaceutically acceptable carrier, diluent, or excipient.

84. A method of treating a disease or disorder, comprising administering a therapeutically effective amount of a compound of any one of claims 1-82, or the pharmaceutical composition of claim 83, to a subject in need thereof.

85. The method of claim 84, wherein the disease or disorder is cancer.

86. The method of claim 85, wherein the cancer is breast cancer, stomach cancer, lung cancer, ovarian cancer, lymphoma, and bladder cancer.

87. A method for preparing a compound of Structure (I): 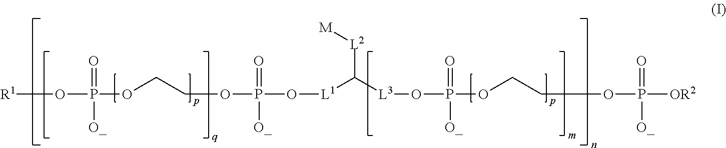
or a pharmaceutically acceptable salt, stereoisomer, or tautomer thereof, wherein
R¹ and R² are independently hydrogen, alkyl, heteroalkyl, Q or a protected form thereof, L′, or have the following structure: 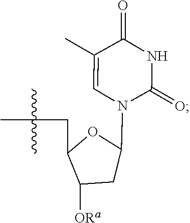
R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0;
p is an integer greater than 0; and
q is an integer greater than 0
the method comprising:
contacting a first compound having the following structure: 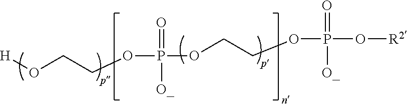
or a salt or tautomer thereof, wherein
R^{2′} comprises a covalent bond to a solid support or solid resin;
n′ is an integer greater than 0;
p′ is an integer greater than 0;
p″ is an integer greater than 0, with a second compound having the following structure: 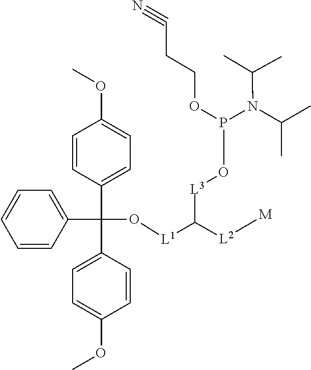
or a salt, tautomer, or stereoisomer thereof,
thereby forming a third compound having the following structure:

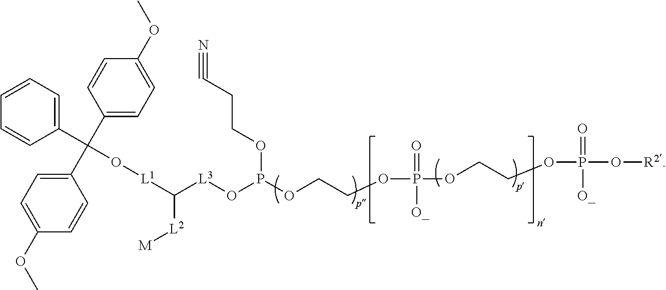

88. A method for preparing a compound of Structure (I): 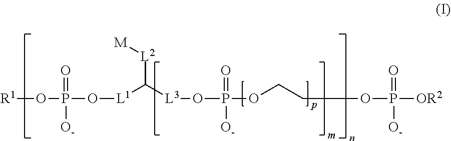
or a pharmaceutically acceptable salt, stereoisomer, or tautomer thereof, wherein
R¹ and R² are independently hydrogen, alkyl, heteroalkyl, Q or a protected form thereof, L′, or have the following structure: 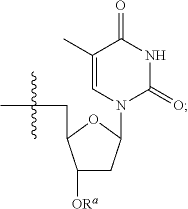
R^{a} is hydrogen or L′;
L¹ and L³ are, at each occurrence, independently a direct bond or an optionally substituted linker;
L² is, at each occurrence, independently a linker;
M is, at each occurrence, independently an anticancer therapeutic;
Q is, at each occurrence, independently a moiety comprising a reactive group, or protected form thereof, capable of forming a covalent bond with a complementary reactive group Q′ on a targeting moiety;
L′ is, at each occurrence, independently a linker comprising a covalent bond to Q, a targeting moiety, a linker comprising a covalent bond to a targeting moiety, a linker comprising a covalent bond to a solid support, a linker comprising a covalent bond to a solid support residue, a solid support residue, a linker comprising a covalent bond to a nucleoside, or a linker comprising a covalent bond to a further compound of Structure (I); and
n is an integer greater than 0;
m is an integer greater than 0; and
p is an integer greater than 0
the method comprising:
contacting a first compound having the following structure: 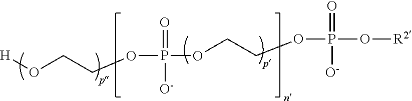
or a salt or tautomer thereof, wherein
R^{2′} comprises a covalent bond to a solid support or solid resin;
n′ is an integer greater than 0;
p′ is an integer greater than 0;
p″ is an integer greater than 0, with a second compound having the following structure: 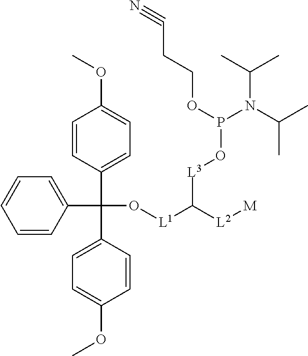
or a salt, tautomer, or stereoisomer thereof,
thereby forming a third compound having the following structure:

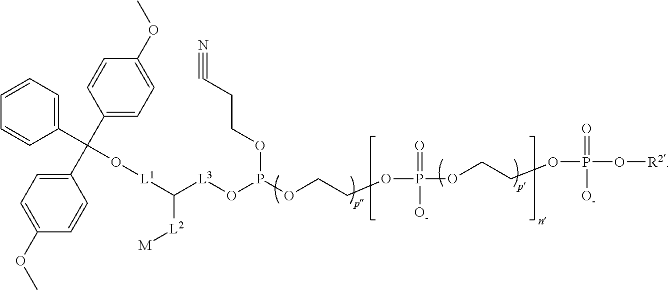

89. The method of claim 87 or 88, wherein the method further comprises oxidizing the third compound by contacting the third compound with iodine, water, and a weak base thereby forming a fourth compound having the following structure:

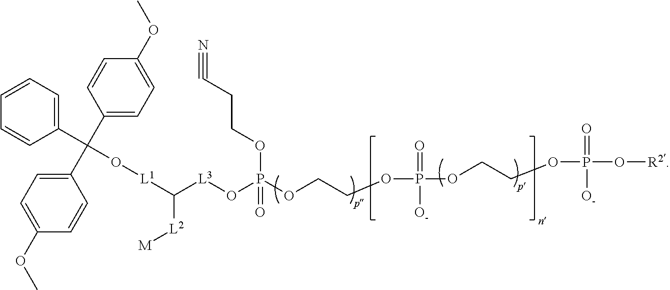

90. The method of claim 89, wherein the weak base is pyridine, lutidine, or collidine.

91. The method of any one of claims 87-90, wherein the method further comprises a deprotection step whereby the fourth compound is contacted with a deprotection solution comprising acid, thereby forming a fifth compound having the following structure:

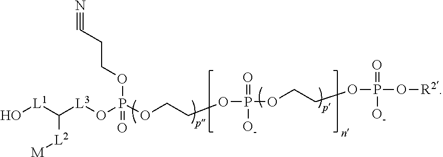

92. The method of claim 91, wherein the acid is chloroacetic acid.

93. The method of claim 91 or 92, wherein the acid is trichloroacetic acid or dichloroacetic acid.

94. The method of any one of claims 91-93, wherein the deprotection solution further comprises dichloromethane or toluene.

95. The method of any one of claims 87-94, wherein the method further comprises removal of 2-cyanoethyl groups.

96. The method of claim 95, wherein the removal of 2-cyanoethyl groups comprises treatment with aqueous ammonia.

97. The method of any one of claims 87-96, wherein the solid support or solid resin is controlled pore glass or macroporous polystyrene.

98. The method of any one of claims 87-97, wherein the method is automated.

99. The method of any one of claims 87-98, wherein a reaction mixture comprising the third compound is contacted with a capping mixture comprising acetic anhydride and 1-methylimidazole.

100. A compound having the following Structure (II): 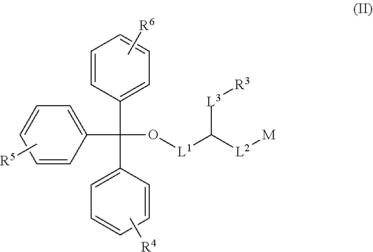
or salt, tautomer, or stereoisomer thereof, wherein:
R³ is H or has the following structure: 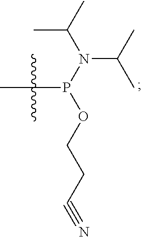 [SMILES: *P(OCCC#N)N(C(C)C)C(C)C]
L¹ a direct bond or an optionally substituted linker;
L² is a linker;
L³ is a direct bond, —O— or —(CH₂)ₘ—O—, wherein m is an integer greater than 0;
R⁴, R⁵, and R⁶ are each independently H or alkoxy; and
M is an anticancer therapeutic.

101. The compound of claim 100, wherein L¹ is a C₁-C₆ alkylene linker.

102. The compound of claim 100 or 101, wherein L¹ is methylene.

103. The compound of any one of claims 100-102, wherein L³ is —O—, C₁-C₆ alkylene-O— linker, or a direct bond.

104. The compound of any one of claims 100-103, wherein L³ is —O—.

105. The compound of any one of claims 100-104, wherein L² heteroalkylene.

106. The compound of any one of claims 100-105, wherein L² comprises oxygen.

107. The compound of any one of claims 100-106, wherein L² comprises the following structure: 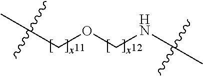
wherein:
x¹¹ and x¹² are each independently an integer greater than 0.

108. The compound of claim 107, wherein x¹¹ is 1, 2, 3, or 4.

109. The compound of claim 107 or 108, wherein x¹² is 2, 3, 4, or 5.

110. The compound of any one of claims 107-109, wherein x¹¹ is 1 or 2 and x¹² is 2, 3, or 4.

111. The compound of any one of claims 100-110, wherein L² comprises one of the following structures:

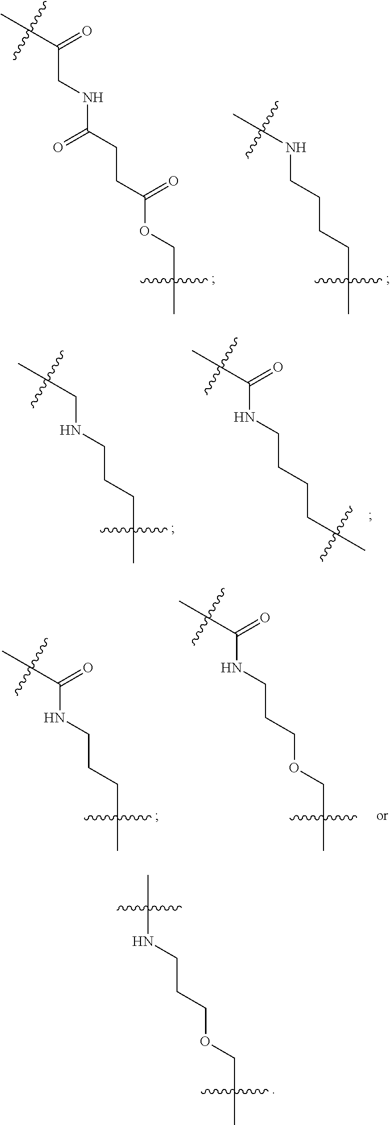

112. The compound of any one of claims 100-111, wherein L² has the following structure: 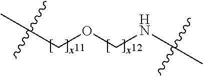
wherein:
x¹¹ and x¹² are each independently an integer greater than 0.

113. The compound of claim 112, wherein x¹¹ is 1, 2, 3, or 4.

114. The compound of claim 112 or 113, wherein x¹² is 2, 3, 4, or 5.

115. The compound of any one of claims 112-114, wherein x¹¹ is 1 or 2 and x¹² is 2, 3, or 4.

116. The compound of any one of claims 100-111, wherein L² further comprises a physiologically cleavable linker.

117. The compound of any one of claims 100-111, wherein L² further comprises an amide bond, an ester bond, a phosphodiester bond, a disulfide bond, a double bond, a triple bond, an ether bond, a hydrazone, an amino acid sequence comprising one or more amino acid residues, a ketone, a diol, a cyano, a nitro, or combinations thereof.

118. The compound of claim 116 or 117, wherein L² comprises an amino acid sequence recognized by a sortase enzyme or cysteine protease.

119. The compound of claim 118, wherein the amino acid sequence is Leu-Pro-X-Thr-Gly, wherein X is any amino acid residue.

120. The compound of any one of claims 100-111, wherein L² comprises one of the following structures:

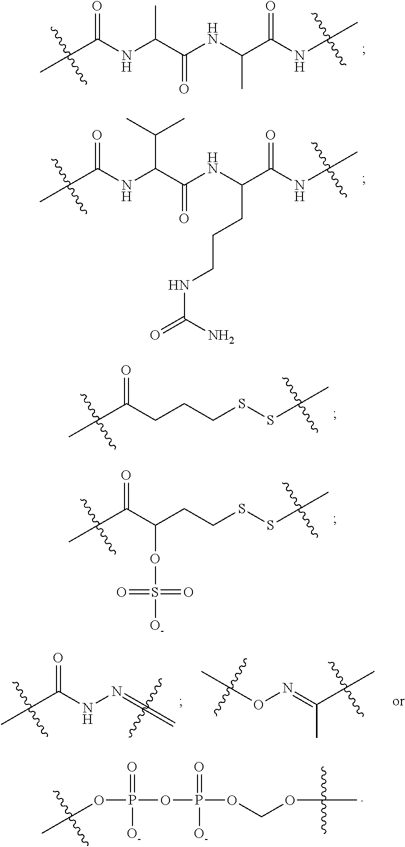

121. The compound of any one of claims 100-111, wherein L² has one of the following structures:

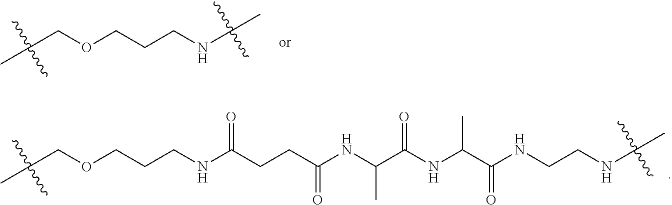 [SMILES: *COCCCN* ; *COCCCNC(=O)CCC(=O)NC(C)C(=O)NC(C)C(=O)NCCN*]

122. The compound of any one of claims 100-121, wherein M is an alkylating agent, an antimetabolite, a microtubule inhibitor, a topoisomerase inhibitor, or a cytotoxic antibiotic.

123. The compound of any one of claims 100-122, wherein M is a camptothecin.

124. The compound of any one of claims 100-123, wherein the compound has the following Structure (IIA): 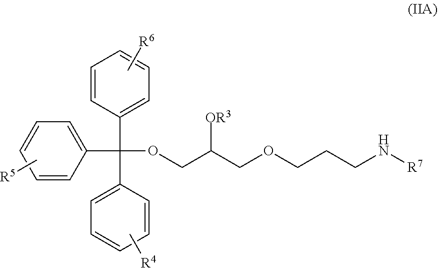
or salt, tautomer, or stereoisomer thereof, wherein:
R³ is H or has the following structure: 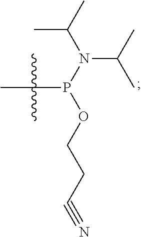 [SMILES: *P(OCCC#N)N(C(C)C)C(C)C]
R⁴, R⁵, and R⁶ are each independently H or alkoxy;
R⁷ has one of the following structures: 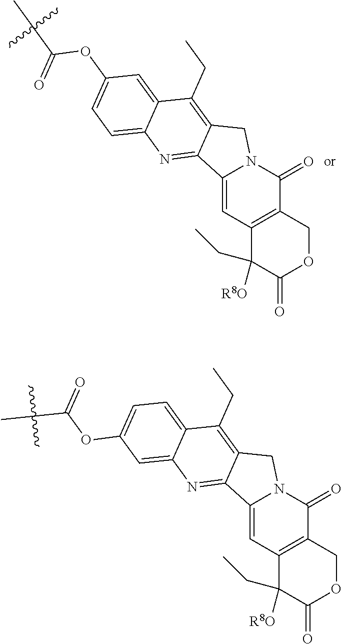
wherein:
R⁸ is H or —C(═O)C(CH₃)₃.

125. The compound of claim 124, wherein the compound has the following Structure (IIB): 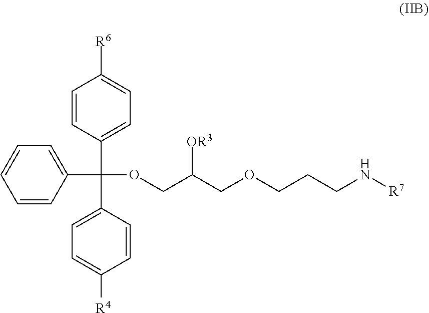
or salt, tautomer, or stereoisomer thereof.

126. The compound of claim 124 or 125, wherein R³ is H.

127. The compound of any one of claims 124-126, wherein R³ has the following structure:

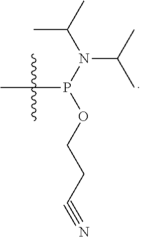 [SMILES: *P(OCCC#N)N(C(C)C)C(C)C]

128. The compound of any one of claims 124-127, wherein R⁴ is alkoxy.

129. The compound of any one of claims 124-128, wherein R⁶ is alkoxy.

130. The compound of any one of claims 124-129, wherein R⁴ is methoxy.

131. The compound of any one of claims 124-130, wherein R⁶ is methoxy.

132. The compound of any one of claims 124-131, wherein R⁷ has one of the following structures:

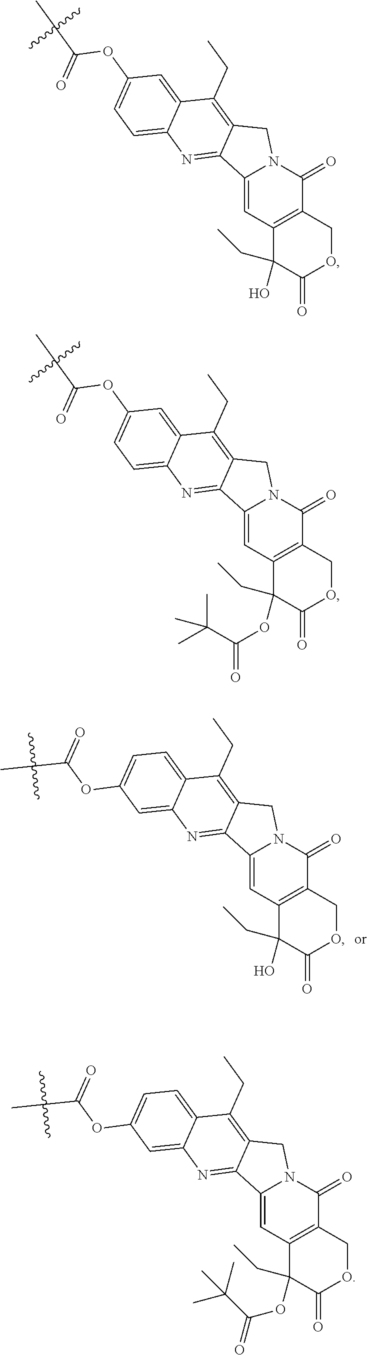 [SMILES: *C(=O)Oc1ccc2nc3c(c(CC)c2c1)Cn1c-3cc2c(c1=O)COC(=O)C2(O)CC ; *C(=O)Oc1ccc2nc3c(c(CC)c2c1)Cn1c-3cc2c(c1=O)COC(=O)C2(CC)OC(=O)C(C)(C)C ; *C(=O)Oc1ccc2c(CC)c3c(nc2c1)-c1cc2c(c(=O)n1C3)COC(=O)C2(O)CC ; *C(=O)Oc1ccc2c(CC)c3c(nc2c1)-c1cc2c(c(=O)n1C3)COC(=O)C2(CC)OC(=O)C(C)(C)C]

133. The compound of any one of claims 124-129, wherein R⁷ has one of the following structures:

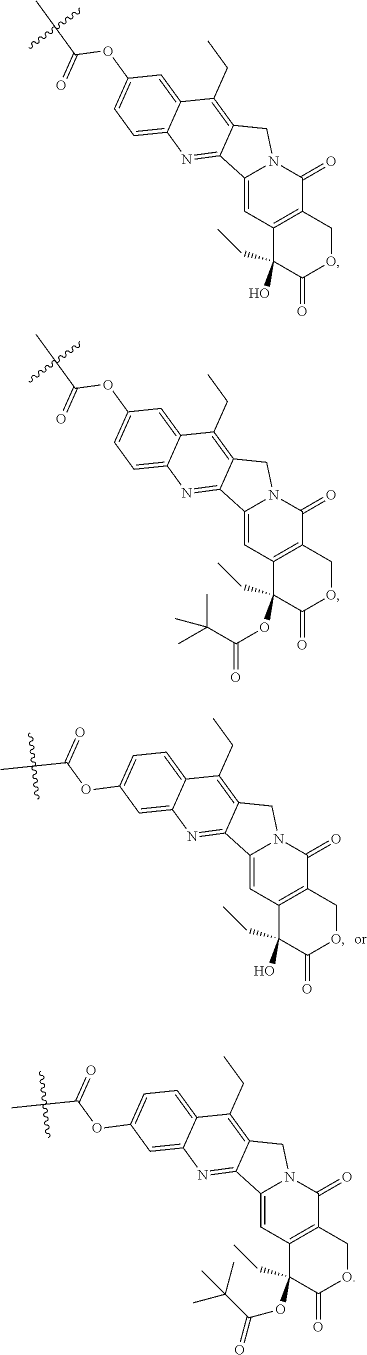 [SMILES: *C(=O)Oc1ccc2nc3c(c(CC)c2c1)Cn1c-3cc2c(c1=O)COC(=O)[C@]2(O)CC ; *C(=O)Oc1ccc2nc3c(c(CC)c2c1)Cn1c-3cc2c(c1=O)COC(=O)[C@@]2(CC)OC(=O)C(C)(C)C ; *C(=O)Oc1ccc2c(CC)c3c(nc2c1)-c1cc2c(c(=O)n1C3)COC(=O)[C@]2(O)CC ; *C(=O)Oc1ccc2c(CC)c3c(nc2c1)-c1cc2c(c(=O)n1C3)COC(=O)[C@@]2(CC)OC(=O)C(C)(C)C]

134. The compound of any one of claims 100-123, wherein the compound has the following Structure (IIA): 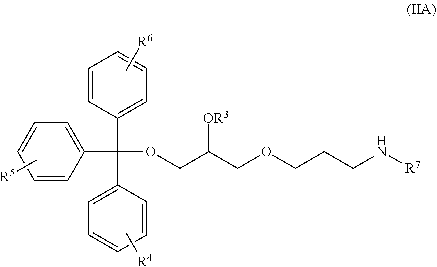
or salt, tautomer, or stereoisomer thereof, wherein:
R³ is H or has the following structure: 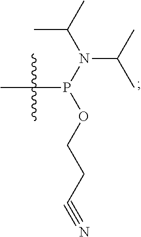 [SMILES: *P(OCCC#N)N(C(C)C)C(C)C]
R⁴, R⁵, and R⁶ are each independently H or alkoxy;
R⁷ has one of the following structures: 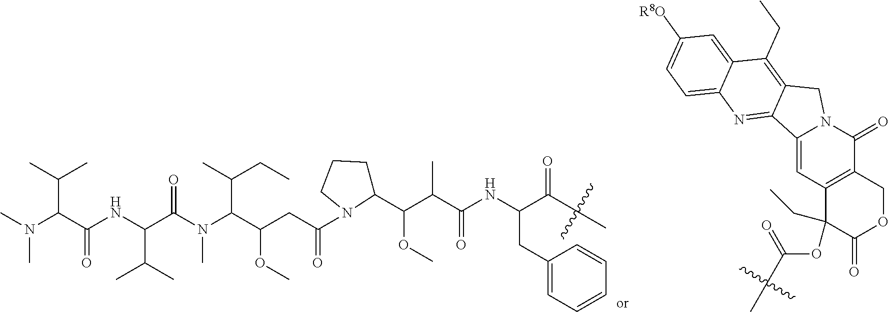
wherein:
R⁸ is H or —C(═O)C(CH₃)₃.

135. The compound of claim 134, wherein the compound has the following Structure (IIB): 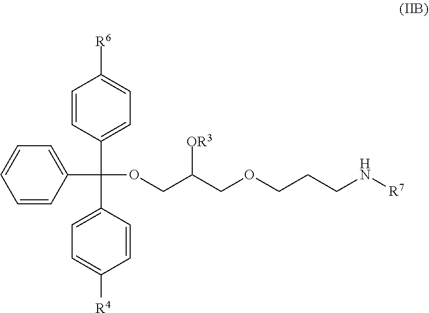
or salt, tautomer, or stereoisomer thereof.

136. The compound of claim 134 or 135, wherein R³ is H.

137. The compound of any one of claims 134-136, wherein R³ has the following structure:

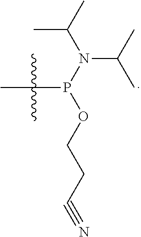 [SMILES: *P(OCCC#N)N(C(C)C)C(C)C]

138. The compound of any one of claims 134-137, wherein R⁴ is alkoxy.

139. The compound of any one of claims 134-138, wherein R⁶ is alkoxy.

140. The compound of any one of claims 134-139, wherein R⁴ is methoxy.

141. The compound of any one of claims 134-140, wherein R⁶ is methoxy.

142. The compound of any one of claims 134-141, wherein R⁷ has the following structure:

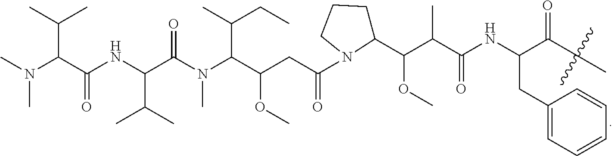 [SMILES: *C(=O)C(Cc1ccccc1)NC(=O)C(C)C(OC)C1CCCN1C(=O)CC(OC)C(C(C)CC)N(C)C(=O)C(NC(=O)C(C(C)C)N(C)C)C(C)C]

143. The compound of any one of claims 134-142, wherein R⁷ has the following structure:

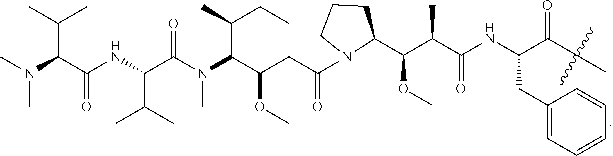

144. The compound of any one of claims 134-141, wherein R⁷ has one of the following structures:

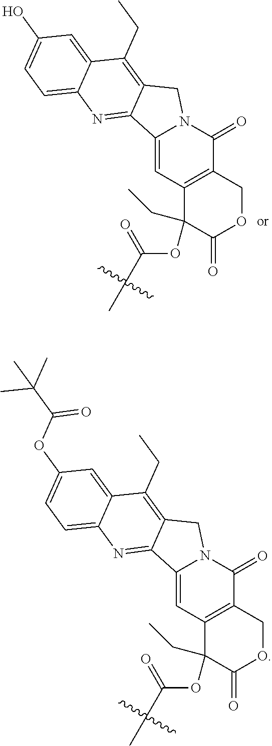 [SMILES: *C(=O)OC1(CC)C(=O)OCc2c1cc1n(c2=O)Cc2c-1nc1ccc(O)cc1c2CC ; *C(=O)OC1(CC)C(=O)OCc2c1cc1n(c2=O)Cc2c-1nc1ccc(OC(=O)C(C)(C)C)cc1c2CC]

145. The compound of any one of claims 134-141, wherein R⁷ has one of the following structures:

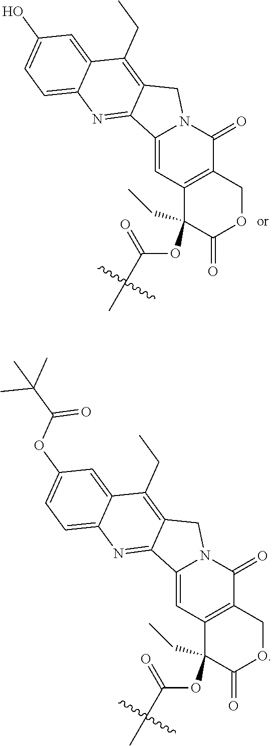 [SMILES: *C(=O)O[C@]1(CC)C(=O)OCc2c1cc1n(c2=O)Cc2c-1nc1ccc(O)cc1c2CC ; *C(=O)O[C@]1(CC)C(=O)OCc2c1cc1n(c2=O)Cc2c-1nc1ccc(OC(=O)C(C)(C)C)cc1c2CC]

146. A compound having one of the structures in Table 2 or a salt, stereoisomer, or tautomer thereof.
